(19) United States
(12) Patent Application Publication
(10) Pub. No.: US 2024/0050366 A1
(43) Pub. Date: Feb. 15, 2024

(54) MEANS AND METHODS OF PREVENTING, TREATING AND DETECTING INFECTIONS

(71) Applicants: SOLYPLUS GMBH, Haselund (DE); LEOPOLD GMBH, Berlin (DE)

(72) Inventors: Andreas VOIGT, Berlin (DE); Richard Dolph ANDERSEN, Berlin (DE); Karl SKRINER, Berlin (DE); Joachim BENDER, Haselund-Brook (DE)

(73) Assignees: SOLYPLUS GMBH, Haselund (DE); LEOPOLD GMBH, Berlin (DE)

(21) Appl. No.: 18/258,861

(22) PCT Filed: Oct. 21, 2021
(86) PCT No.: PCT/EP2021/079199

§ 371 Date: Jun. 22, 2023

(30) Foreign Application Priority Data
Dec. 22, 2020 (EP) 20216717.7
Apr. 14, 2021 (WO) PCT/EP2021/059643

(51) Int. Cl. A61K 9/68; A61K 31/722; C07K 16/10; A61K 9/00; A61K 47/36; G01N 33/569

(52) U.S. Cl. CPC A61K 9/0058; A61K 31/722; C07K 16/1003; A61K 9/006; A61K 47/36; G01N 33/56983; G01N 2333/165

(57) ABSTRACT

Compounds and compositions, their use and methods using the compounds and compositions for preventing an infection, e.g. an infection by a virus or bacterium, or treating an infection, e.g. a virus or bacterial infection, or treating a disease caused by an infection, e.g. a virus or bacterial infection, and a method of increasing the binding of virus neutralizing antibodies, an in vitro method of detecting virus-specific antibodies in a sample obtained from a subject, a compound for use in a method of detecting of virus-specific antibodies in a subject, and a kit for the detection of virus-specific antibodies.

### FIELD

The present invention relates to compounds and compositions, their use and methods using the compounds and compositions for preventing an infection, e.g. an infection by a virus or bacterium, or treating an infection, e.g. a virus or bacterial infection, or treating a disease caused by an infection, e.g. a virus or bacterial infection, and a method of increasing the binding of virus neutralizing antibodies, an in vitro method of detecting virus-specific antibodies in a sample obtained from a subject, a compound for use in a method of detecting of virus-specific antibodies in a subject, and a kit for the detection of virus-specific antibodies.

### BACKGROUND

Viruses and bacteria, infections by viruses and bacteria, and diseases caused by virus or bacterial infections remain a continued and ever evolving threat to health and life. Recently, this has been shown by the global COVID-19 pandemic caused by the virus SARS-CoV-2.

Consequently, there is an ongoing need to provide alternative or improved compositions and methods for treatment and/or prevention of diseases caused by viruses and bacteria. In particular, there is a need to increase the performance of transferring research results to a level of generally accepted applicability.

### SUMMARY

Accordingly, the present invention relates to a method of increasing the binding of virus neutralizing antibodies, the method comprising contacting a fluid that contains a virus and virus neutralizing antibodies, wherein the virus neutralizing antibodies bind to said virus, with a compound selected from the group consisting of chitosan or a salt thereof, galactose, mannose, caffeine, and any combination thereof.

The present invention also relates to an in vitro method of detecting virus-specific antibodies in a sample obtained from a subject, the method comprising contacting a fluid that contains virus-specific antibodies with virus protein under conditions that allow binding of the virus-specific antibody to the virus protein, with a compound selected from the group consisting of chitosan or a salt thereof, galactose, mannose, caffeine, and any combination thereof, thereby detecting said virus-specific antibodies.

The present invention also relates to a compound selected from the group consisting of chitosan or a salt thereof, galactose, mannose, caffeine, and any combination thereof for use in a method of detecting of virus-specific antibodies in a subject.

The present invention also relates to kit for the detection of virus-specific antibodies, the kit comprising a virus protein and a compound selected from the group consisting of chitosan or a salt thereof, galactose, mannose, caffeine, and any combination thereof.

Preferably, in some embodiments the present invention relates to a solid composition for use in preventing an infection by a virus or bacterium, or treating a virus or bacterial infection, or treating a disease caused by a virus or bacterial infection, the composition comprising a compound selected from the group consisting of chitosan or a salt thereof, galactose, mannose, caffeine, and any combination thereof.

Preferably, in some embodiments the present invention also relates to a non-medical use of a solid composition for preventing an infection by a virus or bacterium, wherein the solid composition is as defined herein in accordance with the invention.

Preferably, in some embodiments the present invention also relates to a method of preventing an infection by a virus or bacterium, or treating a virus or bacterial infection, or treating a disease caused by a virus or bacterial infection by the use of a solid composition, wherein the solid composition is as defined herein in accordance with the invention.

Preferably, in some embodiments the present invention also relates to a chewing gum comprising a compound selected from the group consisting of chitosan or a salt thereof, galactose, mannose, caffeine, and any combination thereof.

Preferably, in some embodiments the present invention also relates to an edible composition comprising a compound selected from the group consisting of chitosan or a salt thereof.

Preferably, in some embodiments the present invention also relates to a composition suitable for oral, nasal or bronchial administration, the composition comprising a microparticle, wherein the microparticle comprises chitosan or a salt thereof.

Preferably, in some embodiments the present invention also relates to a sustained delivery system comprising chitosan or a salt thereof.

Preferably, in some embodiments the present invention also relates to a composition suitable for treating a surface or skin, the composition comprising a microparticle, wherein the microparticle comprises chitosan or a salt thereof.

Preferably, in some embodiments the present invention also relates to a method of treating a surface or skin, the method comprising contacting the surface or skin with the composition as defined herein in accordance with the invention.

The present invention also relates to a use of a composition as defined herein in accordance with the invention for inactivating a virus or bacterium.

The present invention also relates to a method of increasing the binding of virus neutralizing antibodies, the method comprising contacting a fluid that contains a virus and virus neutralizing antibodies, wherein the virus neutralizing antibodies bind to said virus, with a compound selected from the group consisting of chitosan or a salt thereof, galactose, mannose, caffeine, and any combination thereof.

The present invention also relates to a compound selected from the group consisting of chitosan or a salt thereof, galactose, mannose, caffeine, and any combination thereof for use in a method of increasing the binding of virus neutralizing antibodies.

The present invention also relates to a method of treating a patient being infected with a virus, the method comprising contacting a fluid that contains a virus and virus neutralizing antibodies, wherein the virus neutralizing antibodies bind to said virus, with a compound selected from the group consisting of chitosan or a salt thereof, galactose, mannose, caffeine, and any combination thereof, wherein said compound increases the binding of virus neutralizing antibodies.

### BRIEF DESCRIPTION OF THE FIGURES

FIG. 1, panel upper left shows tubes including chewing gum probes 236.03 SPF1490 and 0.23 mg/ml 236.02 SPF1494 after 5 days in PBS or saliva at 4° C. Panel upper right shows a 96 well plate of a chitosan ELISA negative controls, which negative controls are showing no detection of chitosan after rinsing the mouth 3× with water and testing chitosan levels. Panel lower left shows a 96 well plate with chitosan ELISA standard concentrations from 10 mg/ml to 0.046 mg/ml. Panel lower right shows a 96 well plate of a chitosan ELISA test sample.

FIGS. 2A, 2B, and 2C show the chitosan release. 2 A shows the chitosan release of four persons (two women/two men) chewing of the chewing gum products 236.03 SPF1490 and 236.02 SPF1494 measured with a chitosan ELISA. 2 B shows the mean level of chitosan release of four persons (two women/two men) chewing of the chewing gum products 236.03 SPF1490 and 236.02 SPF1494 measured with a chitosan ELISA. 2 C shows the total release of chitosan measured by a Chitosan ELISA.

FIG. 3 shows an overview of different MST experiments.

FIG. 4 shows an MST experiment. Therein the concentration of RED (a Cy5 derivative obtained from NanoTemper Technologies, Munich, Germany) labelled (via Snap tag) S protein monomer (20 nM) has been kept constant, while the concentration of non-labeled chitosan was varied between “0.5 μM-0.2 nM” arbitrary units. The assay was performed in PBS containing 0.05% Tween 20 and 50% negative human serum #SK (the titration of the ligand was performed in the negative serum (SK)). After a short incubation the samples were loaded into Monolith™ NT.115 premium capillaries and the MST analysis was performed using a Monolith NT.115. Concentrations on the x-axis are plotted in nM. A binding could be detected.

FIG. 5 shows an MST experiment. Therein the concentration of RED labeled Covid-peptide (10 nM) has been kept constant, while the concentration of non-labeled human ACE2 was varied between 315 nM-0.009 nM. The assay was performed in PBS containing 0.05% Tween20 and 50% negative human serum #MJ (the titration of the ligand was performed in the negative serum). After a short incubation the samples were loaded into Monolith NT.115 premium capillaries and the MST analysis was performed using a Monolith NT.115. Concentrations on the x-axis are plotted in nM. A K_{d} of 43 nM was determined for this interaction.

FIG. 6 shows an MST experiment. Therein the concentration of RED labeled (via the Snap tag) S protein monomer (20 nM) has been kept constant, while the concentration of non-labeled ACE2 with and without Chitosan was varied between “0.5 μM-0.2 nM” arbitrary units. The assay was performed in PBS containing 0.05% Tween20 and 50% negative human serum #SK (the titration of the ligand was performed in the negative serum (SK)). After a short incubation the samples were loaded into Monolith NT.115 premium capillaries and the MST analysis was performed using a Monolith NT.115. Concentrations on the x-axis are plotted in nM. No binding could be detected.

FIG. 7 shows an MST experiment. Therein the concentration of RED labeled (via the Snap tag) protein monomer (20 nM) has been kept constant, while the concentration of non-labeled D-Galactose was varied between “0.5 μM-0.2 nM” arbitrary units. The assay was performed in PBS containing 0.05% Tween20 and 50% negative human serum #SK (the titration of the ligand was performed in the negative serum SK)). After a short incubation the samples were loaded into Monolith™ NT.115 premium capillaries and the MST analysis was performed using a Monolith NT.115. Concentrations on the x-axis are plotted in nM. A binding could be detected.

FIG. 8 shows an MST experiment. Therein the concentration of RED labeled (via the Snap tag) S protein monomer (20 nM) has been kept constant, while the concentration of non labeled mannose was varied between “0.5 μM-0.2 nM” arbitrary units. The assay was performed in PBS containing 0.05% Tween20 and 50% negative human serum #SK (the titration of the ligand was performed in the negative serum (SK)). After a short incubation the samples were loaded into Monolith NT.115 premium capillaries and the MST analysis was performed using a MonolithNT.115. Concentrations on the x-axis are plotted in nM. A binding could be detected.

FIG. 9 shows an MST experiment. Therein the concentration off RED labeled (via the Snap tag) S protein monomer (20 nM) has been kept constant, while the concentration of non-labeled ACE2 and mannose was varied between “0.5 μM-0.2 nM” arbitrary units. The assay was performed in PBS containing 0.05% Tween20 and 50% negative human serum #SK (the titration of the ligand was performed in the negative serum (SK)). After a short incubation the samples were loaded into Monolith NT.115 premium capillaries and the MST analysis was performed using a Monolith NT.115. Concentrations on the x-axis are plotted in nM. No clear binding could be detected.

FIG. 10 shows an MST experiment. Therein the concentration of RED labeled (via the Snap tag) S protein monomer (20 nM) has been kept constant, white the concentration of non-labeled coffeine was varied between “0.5 μM-0.2 nM” arbitrary units. The assay was performed m PBS containing 0.05% Tween20 and 50% negative human serum (the titration of the ligand was performed in the negative serum (SK)). After a short incubation the samples were loaded into Monolith™ NT.115 premium capillaries and the MST analysis was performed using a Monolith NT.115. Concentrations on the x-axis are plotted in nM. A binding could be detected.

FIG. 11 shows an MST experiment. Therein the concentration of RED labeled (via the Snap tag) S protein monomer (20 nM) has been kept constant, while the concentration of non-labeled coffeine was varied between “0.5 μM-0.2 nM” arbitrary units in the presence of ACE2. The assay was performed in PBS containing 0.05% Tween20 and 50% negative human serum #SK (the titration of the ligand was performed in the negative serum (SK)). After a short incubation the samples were loaded into Monolith™ NT.115 premium capillaries and the MST analysis was performed using a Monolith NT.115. Concentrations on the x-axis are plotted in nM. No binding could be detected.

FIG. 12 shows an MST experiment. Therein the concentration of RED labeled (via the Snap tag) S protein monomer (20 nM) has been kept constant, while the concentration of non-labeled L-Arabinose was varied between “0.5 μM-0.2 nM” arbitrary units. The assay was performed in PBS containing 0.05% Tween20 and 50% negative human serum #SK (the titration of the ligand was performed in the negative serum (SK)). After a short incubation the samples were loaded into Monolith™ NT.115 premium capillaries and the MST analysis was performed using a Monolith NT.115. Concentrations on the x-axis are plotted in nM. No binding could be detected.

FIG. 13 shows the binding of monoclonal SARS-CoV-2 neutralising antibodies to RBD-peptide (triplicate measurement).

FIG. 14 shows the binding of human and mouse monoclonal SARS-CoV-2 neutralising antibodies to SNAP-RBD monomer SNAP-RBD trimer (duplicate measurements).

FIGS. 15A and 15B show an enhancement of binding of SARS-CoV-2 binding antibody sera, A shows the median elevation in form of the change in percent of binding enhancement of serum antibodies concerning the binding to SARS-COV-02 RBD-protein in comparison to saliva control without chitosan. 15 B shows the median elevation in form of the change in percent of binding enhancement of serum antibodies concerning the binding to SARS-COV-02 RBD-protein in comparison to saliva control without chitosan.

FIG. 16 schematically depicts chitosan interacting with viruses and cell surfaces under pharynx mucosa, in effect reducing the infection rate. This schematic picture does not take into account any real size relations.

FIGS. 17A and 17B show an enhancement of binding of SARS-CoV-2 binding antibody sera, 17 A shows the median elevation in form of the change in percent of binding enhancement of serum antibodies concerning the binding to SARS-COV-02 N-protein in comparison to saliva control without chitosan. 17 B shows the median elevation in form of the change in percent of binding enhancement of serum antibodies concerning the binding to SARS-COV-02 N-protein in comparison to saliva control without chitosan.

FIG. 18 shows the RBD peptide sequence.

FIG. 19 shows an overview of the RBD peptide within the S1 protein of the S protein of SARS CoV-1 (upper row). The RBD peptide sequence and the RBD peptide, RBD monomer protein with SNAP-Tag, and RBD Timer protein with SNAP-Tag (in the middle). Schematic illustration of chitosan bound to RBD peptide and chitosan bound to RBD monomer protein (lower row).

FIG. 20 shows a schematic illustration of the blocking principle via chitosan. Chitosan inhibits the binding of the RBD protein of SARS-CoV-2 with the ACE2 receptor on the cell surface of the host cell (left side). Chitosan binds to the RBD protein and thereby inhibits binding of the virus RBD to the ACE2 receptor and inhibits in the following viral entry into the host cell (right side).

FIG. 21 shows the amino acid sequence of the S protein SARS-CoV-2 and ACE2 binding region.

FIG. 22 shows the amino acid sequence of the RBD monomer protein (left side) and a schematic illustration of the RBD monomer with SNAP-Tag (right side).

FIG. 23 shows the amino acid sequence of the RBD trimer protein (left side) and a schematic illustration of the RBD trimer with SNAP-Tag (right side).

FIG. 24 shows a synthetic RBD peptide sequence of SARS-CoV-2 with fluorophore-carboxylic acid at the N-terminus, and further illustrating that said peptide is equipped with cysteine—C/C which provide the possibility for forming disulfide bridges.

FIG. 25 shows the results of an anti-SARS-CoV-2 S-RBD ELISA assay. The results of said ELISA assay show antibody level characterization of a Covid-19 cohort and controls with S-RBD protein from a known ELISA with four modified Standard Operating Procedures (SOPs). ODs are shown of 15 COVID-19 sera each and 11 control sera (11 ANA; antinuclear antibody positive). ROC analysis has been performed with the ANA disease control cut offs. Modification 1 and 2 (row 1 and 2): addition of 1 mg/ml Chitosan lactate incubation time 30 min serum dilution (1:200): standard assay: without Chitosan (row 3); Modification 1 and 2 (row 4 and 5): addition of 1 mg/ml Chitosan lactate incubation time 60 min serum dilution (1:100); standard assay without chitosan (row 6); row 7 control sera 30 min serum dilution (1:200); row 8 control sera 60 min serum dilution (1:100); row 9 standard without chitosan.

### BRIEF DESCRIPTION OF THE SEQUENCES

SEQ ID NO:1 is a SARS-CoV-2 RBD peptide sequence from position 436 to 502.

SEQ ID NO:2 is a SARS-CoV-2 RBD peptide sequence from position 426 to 511.

SEQ ID NO:3 is the of the SARS-CoV-2 spike protein.

SEQ ID NO:4 is a SARS-CoV-2 RBD as monomer including a SNAP TAG at the N-terminus.

SEQ ID NO:5 is a SARS-CoV-2 RBD as trimer including a SNAP TAG at the N-terminus.

SEQ ID NO:6 is a SARS-CoV-2 nucleocapsid phosphoprotein sequence

SEQ ID NO:7 is a SARS-CoV-2 spike protein sequence

### DETAILED DESCRIPTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments described throughout the specification should not be construed to limit the present invention to only the explicitly described embodiments. This description should be understood to support and encompass embodiments which combine the explicitly described embodiments with any number of the disclosed and/or preferred elements. Furthermore, any permutations and combinations of all elements described herein should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. When used herein the term “comprising” can be substituted with the term “containing” or “including” or sometimes when used herein with the term “having”. When used herein “consisting of” excludes any element, step, or ingredient not specified.

The terms “a” and “an” and “the” and similar reference used in the context of describing the invention (especially in the context of the claims) are to be construed to cover both the singular and the plural, unless otherwise indicated herein or clearly contradicted by context. The person skilled in the art is aware that the terms “a” or “an”, as used in the present application, may, depending on the situation, mean “one (1)” “one (1) or more” or “at least one (1)”. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless otherwise indicated, the term “at least” preceding a series of elements is to be understood to refer to every element in the series. The term “at least one” refers to one or more such as one, two, three, four, five, six, seven, eight, nine, ten and more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

When used herein “consisting of” excludes any element, step, or ingredient not specified in the claim element. When used herein, “consisting essentially of” does not exclude materials or steps that do not materially affect the basic and novel characteristics of the claim.

The term “including” means “including but not limited to”. “Including” and “including but not limited to” are used interchangeably.

The term “about” means plus or minus 20%, preferably plus or minus 10%, more preferably plus or minus 5%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

The following general definitions apply throughout the present specification, unless stated to the contrary.

“Chitosan”, as used herein, is a linear polysaccharide composed of randomly distributed β-(1→4)-linked D-glucosamine (deacetylated unit) and N-acetyl-D-glucosamine (acetylated unit). It is made, for example, by treating the chitin shells of shrimp and/or other crustaceans with an alkaline substance, such as sodium hydroxide. Chitosan is produced commercially by deacetylation of chitin, which is the structural element in the exoskeleton of crustaceans (such as crabs and shrimp) and cell walls of fungi. Accordingly, as non-limiting examples, crustaceans (e.g. shrimps), fungi, mushrooms, or insects can be employed as a source for chitosan as used herein. Also, chitosan produced by biotechnological fabrication methods can be used. The degree of deacetylation (% DD) can be determined by NMR spectroscopy, and the % DD in commercial chitosans ranges from 30 to 100%. On average, the molecular weight of commercially produced chitosan is 2000 to 3000000 daltons. A common method for the treating of chitosan is the deacetylation of chitin using sodium hydroxide in excess as a reagent and water as a solvent. The amino group in chitosan has a pKa value of ˜6.5, which leads to significant protonation in neutral solution, increasing with increased acidity (decreased pH) and the % DA-value. This makes chitosan water-soluble and permits binding to negatively charged surfaces such as mucosal membranes. Accordingly, as used herein, chitosan can be characterized by the following general formula:

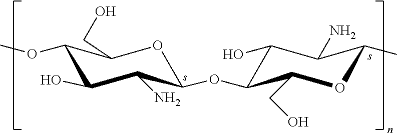

For the purposes of the present disclosure, any chitosan known to a person skilled in the art can be used. As a merely illustrating, non-limiting example, a chitosan which can be used in any embodiment of this specification may have a molecular weight of above 2000 Da. The chitosan may have a molecular weight of above 10000 Da. The chitosan may have a molecular weight of above 20000 Da. The chitosan may have a molecular weight of above 30000 Da. The chitosan may have a molecular weight of above 50000 Da. In addition, or alternatively, the chitosan may have a molecular weight below 3000000 Da. The chitosan may have a molecular weight below 2000000 Da. The chitosan may have a molecular weight below 1000000 Da. The chitosan may have a molecular weight below 500000 Da. The chitosan may have a molecular weight below 250000 Da. The chitosan may have a molecular weight of below 100000 Da. In some embodiments, the chitosan has a molecular weight of from 20000 to 460000 Da. Also, an oligochitosan having a molecular weight of below 20000 Da may be used; decreasing the molecular mass of chitosan leads to an increase of the solubility in water and/or alkaline solution. In addition or alternatively, a chitosan used in any embodiment of this specification may have a degree of deacetylation of 60% or more. The chitosan may have a degree of deacetylation of 70% or more. The chitosan may have a degree of deacetylation of 80% or more. The chitosan may have a degree of deacetylation of 90% or more. The chitosan may have a degree of deacetylation of 95% or more. In any one of the embodiments described herein, the chitosan may have a degree of deacetylation of from 30% to 60%. The chitosan or the salt thereof may have a degree of deacetylation of from 30% to 60%. The chitosan may have a degree of deacetylation of from 35% to 55%. The chitosan may have a degree of deacetylation of from 40% to 50%. Preferably, chitosan having a medium degree of acetylation, e.g. of 40 to 50%, has an increased solubility in water, even under mildly basic conditions, e.g. up to pH 9.

In order to increase solubility of the chitosan in water, in any embodiment of the present specification chitosan may be formulated with an acid. Preferably, the acid is selected from the group consisting of, but not limited to, citric acid, acetic acid, ascorbic acid, malic acid, tartaric acid, any fruit acid, and/or a food acidulant, and any combination thereof. More preferably, the acid is citric acid. The acid may lower the pH so that at least part of the chitosan is converted into its cationic form by protonation so that solubility of the chitosan in water can be increased. Accordingly, any composition described herein comprising chitosan may further comprise an acid.

Also, in any one of the embodiments described herein a salt of chitosan may be used. Any salt of chitosan may be used. A person skilled in the art knows to select suitable salts of chitosan for the purposes described herein. In particular, the salt of chitosan may be a biocompatible salt of chitosan. A person skilled in the art knows to select a salt of chitosan which is biocompatible. Accordingly, the salt of chitosan is not particularly limited and may include, as illustrative, non-limiting examples, a lactate, an acetate, a hydrochloride, a fumarate, a citrate, a maleate, a malate, an ascorbate or a tartrate of chitosan. The salt of chitosan may be chitosan lactate. The salt of chitosan may be chitosan acetate. The salt of chitosan may be chitosan hydrochloride. Salts of chitosan are usually well-soluble in water. The molecular weights and degrees of deacetylation described for chitosan equally apply to a salt of chitosan. Accordingly, a salt of chitosan used in any embodiment of this specification may have a molecular weight of above 2000 Da. The salt of chitosan may have a molecular weight of above 10000 Da. The salt of chitosan may have a molecular weight of above 20000 Da. The salt of chitosan may have a molecular weight of above 30000 Da. The salt of chitosan may have a molecular weight of above 50000 Da. In addition, or alternatively, the salt of chitosan may have a molecular weight below 3000000 Da. The salt of chitosan may have a molecular weight below 2000000 Da. The salt of chitosan may have a molecular weight below 1000000 Da. The salt of chitosan may have a molecular weight below 500000 Da. The salt of chitosan may have a molecular weight below 250000 Da. The salt of chitosan may have a molecular weight of below 100000 Da. In some embodiments, the salt of chitosan has a molecular weight of from 20000 to 460000 Da. Also, a salt of an oligochitosan having a molecular weight of below 20000 Da may be used. In addition or alternatively, a salt of chitosan used in any embodiment of this specification may have a degree of deacetylation of 60% or more. The salt of chitosan may have a degree of deacetylation of 70% or more. The salt of chitosan may have a degree of deacetylation of 80% or more. The salt of chitosan may have a degree of deacetylation of 90% or more. The salt of chitosan may have a degree of deacetylation of 95% or more. In any one of the embodiments described herein, the salt of chitosan may have a degree of deacetylation of from 30% to 60%. The salt of chitosan may have a degree of deacetylation of from 30% to 60%. The salt of chitosan may have a degree of deacetylation of from 35% to 55%. The salt of chitosan may have a degree of deacetylation of from 40% to 50%.

In the embodiments described throughout the present specification, chitosan from any source may be used. As illustrative example, chitosan can be supplied as dried powder, e.g. freeze dried or spray dried. In any embodiment of this specification chitosan from animal sources can be used. In any embodiment of this specification chitosan from fungi may be used. In particular, chitosan from fungi provides a vegan version listed as GRAS in the United States. Accordingly, in any embodiment of the present specification a vegan chitosan may be used, e.g. in a chewing gum.

The term “chitosan”, as used herein, may also include synthetic or semi-synthetic derivatives of chitosan, or salts thereof. Accordingly, the chitosan may be a synthetic or semi-synthetic derivative of chitosan, or a salt thereof. Such derivatives may be modified at the amino groups.

For example, chitosan polymer HTCC or a salt thereof may be used, which is obtainable by reacting chitosan with glycidyltrimethylammonium chloride. Also, a chitosan polymer obtainable by further substituting amino groups of HTCC with hydrophobic groups may be used; as an example, HM-HTCC or a salt thereof may be used, which is obtainable by modifying amino groups of HTCC with n-dodecyl groups. For HTCC and HM-HTCC see, for example, Kaminski K. et al. (2010), Journal of Medicinal Chemistry 53, pages 4141-4147, or WO 2013/172725, which are incorporated herein by reference in their entirety. Also, a positively charged chitosan derivative or a salt thereof, such as e.g. N-palmitoyl-N-monomethyl-N,N-dimethyl-N,N,N-trimethyl-6-O-glycolchitosan (GCPQ) or a salt thereof, may be used as a chitosan derivative. On the other hand, in some embodiments, the chitosan is used as such and not as a derivative of chitosan, or a salt thereof. Accordingly, in some embodiments the chitosan is not a derivative of chitosan or a salt thereof. In some embodiments, the chitosan is not HTCC or a salt thereof. In some embodiments, the chitosan is not HM-HTCC or a salt thereof. In some embodiments, the chitosan is not N-palmitoyl-N-monomethyl-N,N-dimethyl-N,N,N-trimethyl-6-O-glycolchitosan (GCPQ) or a salt thereof. Other derivatives of chitosan, which may be contemplated for the purposes described herein, can be obtained by modifying the amino and/or hydroxy groups to provide, e.g., O-modified chitosan (modified at a hydroxy group), N-modified chitosan (modified at an amino group), or N,O-modified chitosan (modified at both an amino and a hydroxy group). For example, the amino and/or hydroxy groups may be modified partially. Illustrative, non-limiting examples for modified chitosan are phosphorylated, alkylated, benzylated, trimethylated, carboxymethylated, N-succinylated, thiolated, azidated, oxychitin, fluorinated, and sugar-modified chitosan. The chitosan may be also part of a composite, e.g. part of a composite or complex with another polysaccharide, such as e.g. a composite or complex of chitosan with starch, alginate, pectin, carrageenan, xanthan gum, and/or hyaluronic acid. Also, the chitosan may be covalently linked to a carbohydrate, e.g. to another mono-, oligo- or polysaccharide; e.g., chitosan bound to glucose, galactose or mannose. For example, the chitosan may form part of a macromolecular composite or heteropolymer; e.g., chitosan may form part of a heteropolymer with another polysaccharide. Also, the chitosan may be modified with a synthetic polymer or copolymer, e.g. chitosan grafted with a polyethyleneimine or poly(meth)acrylate polymer. Also contemplated herein may be chitosan bound to a peptide, polypeptide or protein. Derivatives of chitosan are generally known to a person skilled in the art, and the skilled person will readily select a derivative of chitosan suitable for the purposes of the present disclosure. Chitosan and derivatives of chitosan are described, e.g., in M. S. R. Rajoka et al., Chitin/chitosan derivatives and their interactions with microorganisms: a comprehensive review and future perspectives, Critical Reviews in Biotechnology, DOI: 10.1080/07388551.2020.1713719; S. Ahmed, S. Ikram (editors), Chitosan: Derivatives, Composites and Applications, Wiley/Scrivener Publishing, Beverly, MA, 2017; Se-Kwon Kim (editor), Chitin, Chitosan, Oligosaccharides and Their Derivatives: Biological Activities and Applications, CRC Press Taylor & Francis Group, Boca Raton, FL, 2011; and Se-Kwon Kim (editor), Chitin and Chitosan Derivatives: Advances in Drug Discovery and Developments, CRC Press Taylor & Francis Group, Boca Raton, FL, 2014, which are all incorporated herein by reference in its entirety.

A “particle” or “particles”, whenever described herein, in general refers to a particle or a plurality of particles having a size between 1 nm (nanometer) and 10 mm (millimeter), e.g. having a size between 1 nm and 8 mm, or e.g. having a size between 1 nm and 2 mm, or e.g. having a size between 1 nm and 1 mm (=1000 μm). In this regard, the terms “particle” and “particles”, i.e. the singular and plural form, can be used interchangeably throughout this specification. Any particle known to a person skilled in the art may be used for the purposes of the present specification. For example, the term “particle” or “particles”, as used herein, may include microparticles or nanoparticles. As also further detailed herein, microparticles may have a size between 0.1 μm and 1000 μm. Nanoparticles may have a size between 1 nm and 1000 nm. Also, particles described herein may have a size between 1 mm and 10 mm, e.g. between 1 mm and 8 mm, e g between 1 mm and 5 mm, or e.g. between 1 mm and 2 mm. The term “particle” or “particles” may include a co-agglomerate of particles; for example, described herein is, inter alia, a co-agglomerate of microparticles or a co-agglomerate of nanoparticles.

A “microparticle” or “microparticles”, whenever described herein, refers to a particle or a plurality of particles having a size between 0.1 and 1000 μm. In this regard, the terms “microparticle” and “microparticles”, i.e. the singular and plural form, can be used interchangeably throughout this specification. As further, merely illustrative example, a microparticle may have a size of from 0.3 to 950 μm. A microparticle may have a size of from 0.5 to 700 μm. A microparticle may have a size of from 0.7 to 300 μm. A microparticle may have a size of from 200 to 300 μm. A microparticle may have a size of from 350 to 450 μm. For example, in a certain amount of particles more than 5% by weight, more than 10% by weight, more than 20% by weight, more than 30% by weight, more than 40% by weight, more than 50% by weight, more than 60% by weight, more than 70% by weight, more than 80% by weight, or even more than 90% by weight, based on 100% of the weight of the total amount of the particles, have a particle size falling within any one of the foregoing ranges.

Any microparticle known to a person skilled in the art may be used for the purposes of the present specification. For example, a microparticle or microparticles may be produced by a conventional drying process, e.g. freeze drying, spray drying or similar methods. Such methods usually result in a broad range of the particle size distribution. Other methods, in particular processes for preparing microparticles as supplied by SolyPlus GmbH, Haselund, Germany, contemplate preparation of microparticles (and also composite microparticles) via a solid form, followed by grinding or cryogenic grinding. By such methods, larger particle sizes, e.g. of from 200 to 300 μm or from 350 to 450 μm can be achieved, and/or narrower particle size distributions. Further separation can be achieved, e.g. by sieving, if desired. For example, processes can be used as described in WO 2019/073361, WO 2019/073362, WO 2019/073363 and WO 2019/073364, which are incorporated herein by reference in their entirety. Another method of preparing microparticles comprises increasing the size of particles, which can be employed as a particle powder. In particular, fluid bed granulation can be used for increasing the particle size. Accordingly, fluid bed granulation can be also used to prepare a co-agglomerate of microparticles. A “co-agglomerate”, as understood by a person skilled in the art, in general refers to a microparticle comprising two or more polymers (e.g. chitosan and another polymer) obtained by increasing the size of a microparticle, e.g. by treating a microparticle comprising a first polymer with a second polymer (e.g., a second polymer in solution) to increase its size, as e.g. described in the following. In particular, in order to prepare a co-agglomerate, different components can be applied in the fluid bed and be incorporated in the co-agglomerate, e.g. two different biopolymers. For example, in order to prepare a co-agglomerate of microparticles, a fluid bed granulation equipment is feeded with hyaluronic acid microparticles; to the fluid bed there is added (e.g. sprayed to) a solution of chitosan; this process is continued up to achieving the target microparticle aggregate size; if desired, the process can be continued with the now obtained microparticle aggregate as starting particle, etc. Preferably, throughout this specification, microparticles can be used which (1) are prepared by a conventional drying processes, (2) are obtained by fluid bed granulation, and/or (3) are prepared by a grinding process or cryogenic grinding process from solid biopolymer materials, e.g. applying processes described herein above and below. As described herein, microparticles and co-agglomerates of microparticles may comprise chitosan or a salt thereof. Optionally, microparticles and co-agglomerates of microparticles may comprise a further polymer or biopolymer, e.g. hyaluronic acid.

A “nanoparticle” or “nanoparticles”, whenever described herein, refers to a particle or a plurality of particles having a size between 1 and 1000 nm. In this regard, the terms “nanoparticle” and “nanoparticles”, i.e. the singular and plural form, can be used interchangeably throughout this specification. As further, merely illustrative example, a nanoparticle may have a size of from 1 to 500 nm. A nanoparticle may have a size of from 1 to 100 nm. A nanoparticle may have a size of from 150 to 800 nm. Any nanoparticle known to a person skilled in the art may be used for the purposes of the present specification. Also described herein is a co-agglomerate of nanoparticles. The structure or composition of a co-agglomerate of nanoparticles may be analogous or similar to the composition of a co-agglomerate of microparticles, as described herein, but with a smaller size. Nanoparticles or a co-agglomerate of nanoparticles can be produced by any suitable method, such as e.g. by analogous or similar methods as described herein for the production of microparticles or co-agglomerates of microparticles.

The inventors have found that chitosan interacts with viruses and bacteria, mucosa components, which are mainly of negative electric charge and the adjacent cell membrane surface, e.g. via electrostatic interaction. This interaction allows that the chitosan inhibits entry of viruses and bacteria into the host cell.

Accordingly, the present invention is described herein, as follows.

### Composition for Use in Preventing an Infection, e.g. an Infection by a Virus or Bacterium, or Treating an Infection, e.g. by a Virus or a Bacterial Infection, or Treating a Disease Caused by an Infection, e.g. by a Virus or a Bacterial Infection

The present invention relates to a composition for use in preventing an infection, or treating an infection, or treating a disease caused by an infection, the composition comprising a compound selected from the group consisting of chitosan or a salt thereof, galactose, mannose, caffeine, and any combination thereof.

Preferably, the infection is a virus, bacterial, protozoan, or mycotic infection. More preferably, the infection is a virus infection.

Preferably, in some embodiments the present invention relates to a composition for use in preventing an infection by a virus or bacterium, or treating a virus or bacterial infection, or treating a disease caused by a virus or bacterial infection, the composition comprising a compound selected from the group consisting of chitosan or a salt thereof, galactose, mannose, caffeine, and any combination thereof.

Preferably, in some embodiments the present invention relates to a composition for use in preventing an infection by a protozoan or fungus, or treating a protozoan or mycotic infection, or treating a disease caused by a protozoan or mycotic infection, the composition comprising a compound selected from the group consisting of chitosan or a salt thereof, galactose, mannose, caffeine, and any combination thereof.

Preferably, in some embodiments the present invention relates to a composition for use in preventing an infection by a virus, the composition comprising a compound selected from the group consisting of chitosan or a salt thereof, galactose, mannose, caffeine, and any combination thereof.

Preferably, in some embodiments the present invention also relates to a composition for use in treating a virus infection, the composition comprising a compound selected from the group consisting of chitosan or a salt thereof, galactose, mannose, caffeine, and any combination thereof.

Preferably, in some embodiments the present invention also relates to a composition for use in treating a disease caused by a virus infection, the composition comprising a compound selected from the group consisting of chitosan or a salt thereof, galactose, mannose, caffeine, and any combination thereof.

Exemplary viruses for the use in preventing an infection by a virus, or treating a virus infection, or treating a disease caused by a virus, as disclosed herein, may be selected from the group consisting of, but are not limited to, a coronavirus, preferably a human-pathogenic coronavirus, an influenza virus, a parainfluenza virus, a rhinovirus, a respiratory syncytial virus [RSV], an adenovirus, an orthomyxivirus, an Epstein-Barr virus, a herpes virus (e.g. herpes simplex), and a measles virus. Other exemplary viruses may be selected from the group consisting of influenza A, HIV-1; hepatitis A, B, C; HPV; EBV; norovirus; herpes simplex; cytomegalovirus; WSSV; RSV; plant virus like; TMV; AMV; TNV; BYMV; PSV; FMV; PVX; acute bee paralyisi virus; IAPV; chronic bee paralysis virus; varroa vectored virus; and in general any RNA and DNA virus. Infection with any virus or bacterium to which the compound (chitosan or salt thereof, galactose, mannose and/or caffeine) can bind can be prevented and/or treated. Illustrative viruses and bacteria include viruses and bacteria inducing diseases in humans, animals, bees and/or plants.

Further, any possible subunit of a virus or part of a virus or virion which is able exert an infection are also examples for the use in preventing an infection by a virus, or treating a virus infection, or treating a disease caused by a virus as disclosed herein.

Further, any possible mutation of a virus which may develop, in particular during virus and host contact and interaction, or evolutionary alteration of a virus are also examples for the use in preventing an infection by a virus, or treating a virus infection, or treating a disease caused by a virus as disclosed herein.

Preferably, the virus is a respiratory virus.

Preferably, the virus is selected from the group consisting of a coronavirus, more preferably a human-pathogenic coronavirus, an influenza virus, a rhinovirus, and a respiratory syncytial virus [RSV].

Preferably, the virus is a coronavirus. As illustrative examples, the family of coronaviridae comprises an alphacoronavirus, a betacoronavirus, a deltacoronavirus, a gammacoronavirus, or an alphaletovirus. Preferably, the coronavirus is a human-pathogenic coronavirus. More preferably, the human-pathogenic coronavirus is a betacoronavirus. Even more preferably, the human-pathogenic coronavirus is selected from the group consisting of SARS-CoV-2, MERS-CoV and SARS-Cov-1.

Accordingly, the human-pathogenic coronavirus may be SARS-CoV-1. The human-pathogenic coronavirus may be MERS-CoV.

Most preferably, the human-pathogenic coronavirus is SARS-CoV-2.

Interestingly, the inventors found out that chitosan or a salt thereof, galactose, mannose or caffeine is able to bind to the RBD peptide of the Spike (S-) protein of SARS-CoV-2. Due to the binding to the RBD peptide, chitosan or a salt thereof, galactose, mannose or caffeine inhibits binding of the virus to the ACE2 receptor on the host cell. Thus, infection of the host cell by SARS-CoV-2 is inhibited by chitosan or a salt thereof, galactose, mannose or caffeine.

The disease caused by a virus infection to be treated by any one of the uses and methods described herein may be any disease caused by a virus, for example, a disease caused by a coronavirus, preferably a human-pathogenic coronavirus, an influenza virus, a parainfluenza virus, a rhinovirus, a respiratory syncytial virus [RSV], an adenovirus, an orthomyxivirus, an Epstein-Barr virus, a herpes virus (e.g. herpes simplex), and a measles virus.

Preferably, the disease is selected from the group consisting of COVID-19, MERS and SARS.

Accordingly, the disease may be SARS. The disease may be MERS.

Most preferably, the disease is COVID-19.

Preferably, in some embodiments the present invention also relates to a composition for use in preventing an infection by a bacterium, the composition comprising a compound selected from the group consisting of chitosan or a salt thereof, galactose, mannose, caffeine, and any combination thereof.

Preferably, in some embodiments the present invention also relates to a composition for use in treating a bacterial infection, the composition comprising a compound selected from the group consisting of chitosan or a salt thereof, galactose, mannose, caffeine, and any combination thereof.

Preferably, in some embodiments the present invention also relates to a composition for use in treating a disease caused by a bacterial infection, the composition comprising a compound selected from the group consisting of chitosan or a salt thereof, galactose, mannose, caffeine, and any combination thereof.

Exemplary bacteria for the use in preventing an infection by a bacterium, or treating a bacterial infection, or treating a disease caused by a bacterium, as described herein, may be selected from the group consisting of, but are not limited to, Streptococcus spec., Haemophilus spec., Bordetella spec., Bacillus spec., Corynebacterium spec., Neisseria spec., Chlamydophila spec., Mycoplasma spec., and Fusobacterium spec. Preferably, the bacterium is a bacterium, which affects the respiratory tract. Herein, such bacterium is also denoted as a respiratory bacterium.

Preferably, in some embodiments the present invention also relates to a composition for use in preventing an infection by a protozoan, the composition comprising a compound selected from the group consisting of chitosan or a salt thereof, galactose, mannose, caffeine, and any combination thereof.

Preferably, in some embodiments the present invention also relates to a composition for use in treating a protozoan infection, the composition comprising a compound selected from the group consisting of chitosan or a salt thereof, galactose, mannose, caffeine, and any combination thereof.

Preferably, in some embodiments the present invention also relates to a composition for use in treating a disease caused by a protozoan infection, the composition comprising a compound selected from the group consisting of chitosan or a salt thereof, galactose, mannose, caffeine, and any combination thereof.

Exemplary protozoa for the use in preventing an infection by a protozoan, or treating a protozoan infection, or treating a disease caused by a protozoa, as described herein, may be selected from the group consisting of, but are not limited to, organism which are formerly classified in the Kingdom Protozoa, that are now classified in the supergroups Excavata, Amoebozoa, SAR, and Archaeplastida. Distinct examples of protozoa causing a protozoan infection are selected from the group consisting of, but are not limited to, Giardia intestinalis, Hexamita salmonis, Histomonas meleagridis, Trichomonas foetus, Dientamoeba fragilis, Trichomonas vaginalis, Leishmania, Trypanosoma cruzi, Trypanosoma brucei rhodensiense, Trypanosoma brucei gambiense, Entamoeba histolytica, Naeglaria, Acanthomoeba, Phytophthora infestans, Balantidium Coli, Toxoplasma gondii, Plasmodium falciparum, Plasmodium vivax, Plasmodium ovale, Plasmodium malariae, and Plasmodium knowlesi.

Preferably, in some embodiments the present invention also relates to a composition for use in preventing an infection by a fungus, the composition comprising a compound selected from the group consisting of chitosan or a salt thereof, galactose, mannose, caffeine, and any combination thereof.

Preferably, in some embodiments the present invention also relates to a composition for use in treating a mycotic infection, the composition comprising a compound selected from the group consisting of chitosan or a salt thereof, galactose, mannose, caffeine, and any combination thereof.

Preferably, in some embodiments the present invention also relates to a composition for use in treating a disease caused by a mycotic infection, the composition comprising a compound selected from the group consisting of chitosan or a salt thereof, galactose, mannose, caffeine, and any combination thereof.

Exemplary fungi for the use in preventing an infection by a fugus, or treating a mycotic infection, or treating a disease caused by a fungus, as described herein, may be selected from the group consisting of, but are not limited to, Candida albicans, Candida stellatoidea, C. tropicalis, C. pseudotropicalis, C. krusei, C. parapsilosis, C. guilliermondii, Aspergillus fumigatus, Aspergillus clavatus, Cryptococcus neoformans, Cryptococcus laurentii, Cryptococcus albidus, C. neoformans, Histoplasma capsulatum, Pneumocystis jirovecii, and Stachybotrys chartarum.

Any one of the uses and methods described herein may in general comprise contacting a bodily fluid with the composition. For example, the bodily fluid may be blood, saliva and a mucous secretion. Preferably, in particular when the virus or bacterium is respiratory virus or bacterium, such as e.g. the virus SARS-CoV-2, any one of the uses and methods described herein may comprise contacting the composition with saliva and/or a mucous secretion. Accordingly, the bodily fluid may be saliva. The bodily fluid may be a mucous secretion. Preferably, in particular when the virus or bacterium is a respiratory virus or bacterium, such as e.g. the virus SARS-CoV-2, the mucous secretion is a nasal secretion and/or a bronchial secretion. When the composition is for use in preventing an infection by a respiratory virus or bacterium, such as e.g. the virus SARS-CoV-2, the composition is preferably delivered to saliva or a mucous secretion of the respiratory tract, e.g. by spraying or gargling, as respiratory viruses after entering the human body are typically present in such bodily fluids. In addition or alternatively, any one of the uses and methods described herein may comprise contacting a mucosa with the composition. In particular, when the virus is a respiratory virus or bacterium, such as e.g. the virus SARS-CoV-2, the mucosa may be selected from the group consisting of mucosa of the nose, mucosa of the pharynx, mucosa of the mouth, and mucosa of the bronchia. Accordingly, the mucosa may be mucosa of the nose. The mucosa may be mucosa of the pharynx. The mucosa may be mucosa of the mouth. The mucosa may be mucosa of the bronchia.

According to the invention, the composition comprises a compound selected from the group consisting of chitosan or a salt thereof, galactose, mannose, caffeine, and any combination thereof. Any one of the compositions described herein can be used in any one of the uses and methods described herein. Accordingly, in particular, any one of the compositions described herein can be used in any one of the uses and methods for preventing an infection by a virus, a bacterium, a protozoan, or a fungus, or treating a virus, bacterial, protozoan, or mycotic infection, or treating a disease caused by a virus, bacterial, protozoan, or mycotic infection as described herein throughout this specification. Preferably, any one of the compositions described herein can be used in any one of the uses and methods for preventing an infection by a virus or treating a virus infection or treating a disease caused by a virus infection as described herein throughout this specification; more preferably wherein the virus is SARS-CoV-2; and/or more preferably wherein the disease is COVID-19.

Accordingly, the compound may be caffeine. The compound may be mannose. The compound may be galactose. Preferably, the compound is chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein in accordance with the invention is any combination of the foregoing compounds. The salt may be in pharmaceutically acceptable quality.

When the compound is chitosan, optionally the composition may further comprise an acid. Preferably, the acid is selected from the group consisting of, but not limited to, citric acid, acetic acid, ascorbic acid, malic acid, tartaric acid, any fruit acid, a food acidulant, and any combination thereof. More preferably, the acid is citric acid. The acid may lower the pH so that at least part of the chitosan is converted into its cationic form. Accordingly, solubility of the chitosan in water can be increased.

Preferably, the composition is a solid composition. As merely illustrative examples, a solid composition may be in the form of a powder, particles, a tablet, a pill, a capsule, etc. The term “solid composition”, whenever used throughout this specification, may also include semi-solid compositions, such as e.g. gels, creams, pastes, ointments, salves, etc.

As an illustrative example, the composition may be a substantially pure preparation of the compound or the combination thereof. Also, the composition may comprise, as illustrative examples, 1% or more, 5% or more, 10 or more, 20% or more, 50% or more, 80% or more, or 90% or more of the compound or the combination thereof.

Preferably, the composition is an edible composition. Any edible composition known to a person skilled in the art may be contemplated, e.g. a food or beverage. In general, as understood by a person skilled in the art, an edible composition is digestible. In the oral cavity, for example when the composition is contacted with the saliva, the compound, e.g. chitosan or a salt thereof, is released from the composition and/or contacted with the virus, bacterium, protozoan or fungus, e.g. the virus SARS-CoV-2. The virus, bacterium, protozoan or fungus, e.g. the virus SARS-CoV-2 is then bound by the compound or the combination thereof, will be swallowed, and is then inactivated in the stomach by gastric acid. Accordingly, infection with the virus or bacterium is prevented. In addition or alternatively, the compound may bind to the virus, bacterium, protozoan or fungus, e.g. SARS-CoV-2, so that binding of the virus to the ACE2 receptor on the host cell is prevented and no infection can take place. Also, such mechanism can be used for the treating of a virus or bacterial infection, e.g. with SARS-CoV-2, and/or for the treating of a disease caused by a virus, bacterial, protozoan or mycotic infection, e.g. COVID-19. The adsorption of chitosan to the surface of a virus, a bacterium, protozoan or fungus does not only prevent or inhibit the successful contact to target cells of the submucosa, but it also provides time to activate the own defense mechanisms of the body.

Preferably, the composition may be a composition suitable for oral, nasal or bronchial administration. Any composition known for oral, nasal or bronchial administration may be contemplated. Accordingly, the composition may be a composition for intranasal or inhaled administration, e.g. local administration to the respiratory tract and/or the bronchia. Also, as illustrative examples, the composition may be a spray or solution for gargling. As described herein, by administering the composition, the virus or bacterium, e.g. SARS-CoV-2, is bound and the chain of infection is interrupted. Also, such mechanism can be used for the treating of a virus, bacterial, protozoan or mycotic infection, e.g. with SARS-Cov-2, and/or for the treating of a disease caused by a virus or bacterial infection, e.g. COVID-19.

Also, the composition may be a pharmaceutical composition. The pharmaceutical composition comprises the compound or combination thereof and may optionally further comprise a pharmaceutically acceptable carrier, excipient or stabilizer, such as those described in Remington's Pharmaceutical Sciences 16^{th} edition, Osol, A. Ed. (1980) or 23^{rd} edition (2020), provided that they do not adversely affect the desired characteristics of the formulation. As used herein, “pharmaceutically acceptable carrier” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, asparagine, 2-phenylalanine, and threonine; sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as glutathione, thioctic acid, sodium thioglycolate, thioglycerol, [alpha]-monothioglycerol, and sodium thio sulfate, low molecular weight proteins, such as human serum albumin, bovine serum albumin, gelatin, or other immunoglobulins; and hydrophilic polymers, such as polyvinylpyrrolidone.

Preferably, the composition is a dietary supplement. In a general, a dietary supplement refers to a manufactured product intended to supplement one's diet. For example, the dietary supplement may be in form of a pill, capsule, tablet, powder or liquid. The dietary supplement may be a chewing gum. Preferably, in particular when the composition is solid, e.g. in form of a powder, the dietary supplement is suitable for being added to a beverage. More preferably, the beverage is selected from the group consisting of tea, coffee, juice, lemonade, milk, cola, a drinkable milk product (e.g. drinkable yoghurt), a beverage obtainable by dissolving a sherbet powder or an effervescent tablet, and an alcoholic beverage. Accordingly, the beverage to which the dietary supplement is added, may be tea. The beverage may be coffee. The beverage may be juice (e.g. a fruit juice). The beverage may be lemonade. The beverage may be milk. The beverage may be cola. The beverage may be a drinkable milk product (e.g. drinkable yoghurt). The beverage may be a beverage obtainable by dissolving a sherbet powder or an effervescent tablet. The beverage may be an alcoholic beverage, e.g. beer or wine.

Other preferred compositions, which are solid, and also edible, may be selected from the group consisting of a chewing gum, a candy, a bonbon, a fruit gum, a chocolate, and a composition for preparing a beverage (e.g. a coffee composition, a tea composition, a sherbet powder, or an effervescent tablet). Accordingly, the composition may be a chewing gum. The composition may be a candy. The composition may be a fruit gum. The composition may be a chocolate. The composition may be a composition for preparing a beverage. The composition may be a coffee composition (e.g. a composition comprising coffee, and further ingredients, e.g. milk powder, flavoring agents, and/or sweeteners, etc.). The composition may be a tea composition (e.g. a composition comprising tea leaves, or other components for a tea, e.g. fruit, and further ingredients, flavoring agents, and/or sweeteners, etc.). The composition may be a sherbet powder. The composition may be an effervescent tablet.

In a preferred embodiment, the composition is a chewing gum. Any chewing described throughout this specification can be used.

The chewing gum comprises the compound selected from the group consisting of chitosan or a salt thereof, galactose, mannose, caffeine, and any combination thereof. Accordingly, the compound may be caffeine. The compound may be mannose. The compound may be galactose. Preferably, the compound is chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein in accordance with the invention is any combination of the foregoing compounds. The salt may be in pharmaceutically acceptable quality.

The inventors have demonstrated that, during chewing the chewing gum, the compound, e.g. chitosan or a salt thereof, will be released to the saliva and/or the oral cavity. Then the compound, e.g. chitosan or a salt thereof, binds to the virus, bacterium, protozoan or fungus, e.g. SARS-CoV-2, will be swallowed, and is then inactivated in the stomach by gastric acid. In addition or alternatively, the compound may bind to the virus or bacterium, e.g. SARS-CoV-2, so that binding of the virus to the ACE2 receptor on the host cell is prevented and so no infection can take place. Also, such mechanism can be used for the treating of a virus, bacterial, protozoan or mycotic infection, e.g. with SARS-CoV-2, and/or for the treating of a virus, bacterial, protozoan or mycotic infection, e.g. COVID-19.

In some embodiments, the chewing gum may comprise chitosan and an acid. Preferably, the acid is selected from the group consisting of, but not limited to, citric acid, acetic acid, ascorbic acid, malic acid, tartaric acid, any fruit acid, a food acidulant, and any combination thereof. More preferably, the acid is citric acid. When chewing, the acid lowers the pH so that at least part of the chitosan is converted into its cationic form. Accordingly, solubility of the chitosan in water is increased.

Also, the composition, which comprises the compound or the combination thereof, may be a beverage, as an example for a liquid composition. The beverage may be selected from the group consisting of tea, coffee, juice, lemonade, milk, cola, a drinkable milk product (e.g. a drinkable yoghurt), a beverage obtainable by dissolving a sherbet powder or an effervescent tablet, and an alcoholic beverage. Accordingly, the beverage which comprises the compound or the combination thereof, may be tea. The beverage may be coffee. The beverage may be juice (e.g. a fruit juice). The beverage may be lemonade. The beverage may be milk. The beverage may be cola. The beverage may be a drinkable milk product (e.g. drinkable yoghurt). The beverage may be a beverage obtainable by dissolving a sherbet powder or an effervescent tablet. The beverage may be an alcoholic beverage, e.g. beer or wine.

In a preferred embodiment, the composition is or comprises a sustained delivery system. Preferably, the sustained delivery system is an oral or buccal sustained delivery system. Also described herein, according to embodiments of the invention, the drug delivery system may comprise a drug delivery implant, e.g. a refillable drug delivery implant. Several implantable devices may be contemplated for this purpose, e.g. a dental prothesis or a prosthesis fixation glue material.

Preferably, the composition is or comprises a solid or semi-solid dosage form. Accordingly, the composition is selected from the group consisting of a film, a layered structure of films, a tablet, a capsule, a gel body (e.g. a gel for application in the oral cavity), a chewing gum, a candy, a bonbon, a fruit gum, and a chocolate. Thus, the composition may be a film. The composition may be a layered structure of films. The composition may be a tablet. The composition may be a capsule. The composition may be a gel body (e.g. a gel for application in the oral cavity). The composition may be a chewing gum. The composition may be a candy. The composition may be a bonbon. The composition may be a fruit gum. The composition may be a chocolate.

Any one of the compositions described herein may comprise a particle. Preferably, the particle is a microparticle or a nanoparticle, more preferably a microparticle. Preferably, the particle, preferably a microparticle or a nanoparticle, more preferably a microparticle, comprises chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is a particle, preferably a microparticle or a nanoparticle, more preferably a microparticle, comprising any combination of the foregoing compounds. The salt may be in pharmaceutically acceptable quality.

In addition to the chitosan or salt thereof, the particle, preferably a microparticle or a nanoparticle, more preferably a microparticle, may comprise a further polymer or biopolymer.

The polymer or biopolymer can include, but is not limited to, one or more of the following substances or classes of substances: proteins, polysaccharides, hydrocarbons, nucleic acids, aptamers, collagen, collagen-n-hydroxysuccinimide, fibrin, gelatin, albumin, alginates, blood plasma proteins, milk proteins, casein, protein-based polymers, hyaluronic acid, pectins, gummi arabicum and other gums, casein, whey proteins, gluten, starch, cellulose, synthetic polymers for pharmaceutical or cosmetic applications, like polylactic acid, polyglycolic acid, cell lysates of plants and microorganisms, copolymers and/or derivatives and/or mixtures and/or chemical modifications of said polymers and any combination thereof, with different material parameters such as chain length or molecular weight. The polymers may be utilized, for example, as excipients (e.g., for the incorporation and processing of active ingredients), as basic formulation substances (e.g., for cosmetics). Preferably, the further biopolymer is hyaluronic acid. Accordingly, in a preferred embodiment, the particle, preferably a microparticle or a nanoparticle, more preferably a microparticle, further to chitosan or a salt thereof, comprises hyaluronic acid.

Any one of the compositions described herein may comprise a co-agglomerate of particles. Preferably, the co-agglomerate is a co-agglomerate of microparticles or a co-agglomerate of nanoparticles; more preferably the co-agglomerate is a co-agglomerate of microparticles. Preferably, the co-agglomerate of particles, preferably a co-agglomerate of microparticles or a co-agglomerate of nanoparticles, more preferably a co-agglomerate of microparticles, comprises chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is a co-agglomerate of particles, preferably a co-agglomerate of microparticles or a co-agglomerate of nanoparticles, more preferably a co-agglomerate of microparticles, comprising any combination of the foregoing compounds. The salt may be in pharmaceutically acceptable quality.

In addition to the chitosan or salt thereof, the co-agglomerate of particles, preferably a co-agglomerate of microparticles or a co-agglomerate of nanoparticles, more preferably a co-agglomerate of microparticles, may comprise a further polymer or biopolymer. The polymer or biopolymer can include, but is not limited to, one or more of the following substances or classes of substances: proteins, polysaccharides, hydrocarbons, nucleic acids, aptamers, collagen, collagen-n-hydroxysuccinimide, fibrin, gelatin, albumin, alginates, blood plasma proteins, milk proteins, casein, protein-based polymers, hyaluronic acid, pectins, gummi arabicum and other gums, casein, whey proteins, gluten, starch, cellulose, synthetic polymers for pharmaceutical or cosmetic applications, like polylactic acid, polyglycolic acid, cell lysates of plants and microorganisms, copolymers and/or derivatives and/or mixtures and/or chemical modifications of said polymers and any combination thereof, with different material parameters such as chain length or molecular weight. The polymers may be utilized, for example, as excipients (e.g., for the incorporation and processing of active ingredients), as basic formulation substances (e.g., for cosmetics). Preferably, the further biopolymer is hyaluronic acid. Accordingly, in a preferred embodiment, the co-agglomerate of particles, preferably a co-agglomerate of microparticles or a co-agglomerate of nanoparticles, more preferably a co-agglomerate of microparticles, further to chitosan or a salt thereof, comprises hyaluronic acid.

The particle, preferably a microparticle or a nanoparticle, more preferably a microparticle, or the co-agglomerate of particles, preferably a co-agglomerate of microparticles, or a co-agglomerate of nanoparticles, more preferably a co-agglomerate of microparticles, may be comprised in a liquid or semi-solid composition. In particular, the particle, preferably a microparticle or a nanoparticle, more preferably a microparticle, or the co-agglomerate of particles, preferably a co-agglomerate of microparticles or a co-agglomerate of nanoparticles, more preferably a co-agglomerate of microparticles, is suspended in a liquid or semi-solid composition. Preferably, the particle, preferably a microparticle or a nanoparticle, more preferably a microparticles, or co-agglomerate of particles, preferably co-agglomerate of microparticles or co-agglomerate of nanoparticles, more preferably co-agglomerate of microparticles, is comprised, in particular suspended, in a composition for spraying or gargling, a mouth rinse, a gel for application to the oral cavity, a nose spray, a nose oil, a nose drop formulation, and a nasal irrigation formulation.

The composition may be comprised in a solid mixture for manufacturing a nasal irrigation solution, a nose drop solution, a nose oil formulation, a solution for spraying or gargling, a mouth rinse, a gel for application to the oral cavity, or a beverage. The composition may be in form of a particle, preferably a microparticle or nanoparticle, more preferably a microparticle.

In any one of the compositions described herein, the chitosan or the salt thereof may have a degree of deacetylation of 60% or more. The chitosan or the salt thereof may have a degree of deacetylation of 70% or more. The chitosan or the salt thereof may have a degree of deacetylation of 80% or more. The chitosan or the salt thereof may have a degree of deacetylation of 90% or more. The chitosan or the salt thereof may have a degree of deacetylation of 95% or more.

In any one of the compositions described herein, the chitosan or the salt thereof may have a degree of deacetylation of from 30% to 60%. the chitosan or the salt thereof may have a degree of deacetylation of from 30% to 60%. The chitosan or the salt thereof may have a degree of deacetylation of from 35% to 55%. The chitosan or the salt thereof may have a degree of deacetylation of from 40% to 50%.

In any one of the compositions described herein, the chitosan or salt thereof may have a molecular weight of from 20000 Da to 460000 Da.

In some embodiments, the composition may comprise an oligochitosan having a molecular weight of below 20000 Da, or a salt of an oligochitosan having a molecular weight of below 20000 Da.

### Non-Medical Use of a Composition for Preventing an Infection, e.g. an Infection by a Virus or Bacterium

The present invention also relates to a non-medical use of a composition for preventing an infection, wherein the composition is any composition as defined herein.

Preferably, the infection is a virus, bacterial, protozoan or mycotic infection. More preferably, the infection is a virus infection.

Preferably, in some embodiments the the present invention also relates to a non-medical use of a composition for preventing an infection by a virus, bacterium, protozoan or fungus, wherein the composition is any composition as defined herein.

Preferably, in some embodiments the present invention also relates to a non-medical use of a composition for preventing an infection by a virus, wherein the composition is any composition as defined herein.

Preferably, in some embodiments the present invention also relates to a non-medical use of a composition for preventing an infection by a bacterium, wherein the composition is any composition as defined herein.

Preferably, in some embodiments the present invention also relates to a non-medical use of a composition for preventing an infection by a protozoan, wherein the composition is any composition as defined herein.

Preferably, in some embodiments the present invention also relates to a non-medical use of a composition for preventing an infection by a fungus, wherein the composition is any composition as defined herein.

Preferably, the composition is a solid composition.

In some embodiments, the composition may be a liquid composition

In the non-medical use, the virus may be any virus defined herein. Preferably, the virus is a respiratory virus. More preferably, the virus is selected from the group consisting of a coronavirus, preferably a human-pathogenic coronavirus, an influenza virus, a rhinovirus, and a respiratory syncytial virus [RSV]. Accordingly, the coronavirus may be a human-pathogenic coronavirus. Even more preferably, the human-pathogenic coronavirus is a betacoronavirus. The human-pathogenic coronavirus may be SARS-CoV-2. The human-pathogenic coronavirus may be MERS-CoV. The human-pathogenic coronavirus may be SARS-CoV-1. Most preferably, the human-pathogenic coronavirus is SARS-CoV-2.

Further, any possible subunit of a virus or part of a virus or virion which is able exert an infection are also examples for the non-medical use in preventing an infection by a virus, as disclosed herein.

Further, any possible mutation of a virus which may develop, in particular during virus and host contact and interaction, or evolutionary alteration of a virus are also examples for the non-medical use in preventing an infection by a virus, as disclosed herein.

Preferably, the use comprises contacting a bodily fluid with the composition. In particular, the bodily fluid is saliva or a mucous secretion. The mucous secretion may be a nasal secretion or a bronchial secretion. Also preferably, the use may comprise contacting a mucosa with the composition. The mucosa may be mucosa of the nose. The mucosa may be mucosa of the pharynx. The mucosa may be mucosa of the mouth. The mucosa may be mucosa of the bronchia.

### Method of Preventing an Infection, e.g. and Infection by a Virus or Bacterium, or Treating an Infection, e.g. a Virus or Bacterial Infection, or Treating a Disease, e.g. a Disease Caused by a Virus or Bacterial Infection

The present invention also relates to a method of preventing an infection, or treating an infection, or treating a disease caused by an infection by the use of a composition, wherein the composition is any composition as defined herein.

Preferably, in some embodiments the present invention also relates to a method of preventing an infection by a virus or bacterium, or treating a virus or bacterial infection, or treating a disease caused by a virus or bacterial infection by the use of a composition, wherein the composition is any composition as defined herein.

Preferably, in some embodiments the present invention also relates to a method of preventing an infection by a protozoan or fungus, or treating a protozoan or mycotic infection, or treating a disease caused by a protozoan or mycotic infection by the use of a composition, wherein the composition is any composition as defined herein.

Preferably, the infection is a virus or bacterial infection. More preferably, the infection is a virus infection.

Preferably, in some embodiments the present invention also relates to a method of preventing an infection by a virus by the use of a composition, wherein the composition is any composition as defined herein.

Preferably, in some embodiments the present invention also relates to a method of treating a virus infection by the use of a composition, wherein the composition is any composition as defined herein.

Preferably, in some embodiments the present invention also relates to a method of treating a disease caused by a virus infection by the use of a composition, wherein the composition is any composition as defined herein.

Preferably, in any one of the methods the composition is a solid composition.

In some embodiments, the composition may be a liquid composition.

In any one of the methods, the virus may be any virus defined herein. Preferably, the virus is a respiratory virus. More preferably, the virus is selected from the group consisting of a coronavirus, preferably a human-pathogenic coronavirus, an influenza virus, a rhinovirus, and a respiratory syncytial virus [RSV]. Accordingly, the coronavirus may be a human-pathogenic coronavirus. Even more preferably, the human-pathogenic coronavirus is a betacoronavirus. The human-pathogenic coronavirus may be SARS-CoV-2. The human-pathogenic coronavirus may be MERS-CoV. The human-pathogenic coronavirus may be SARS-CoV-1. Most preferably, the human-pathogenic coronavirus is SARS-CoV-2. In any one of the methods, the disease may be SARS. The disease may be MERS. Most preferably, the disease is COVID-19.

Further, any possible subunit of a virus or part of a virus or virion which is able exert an infection are also examples which can be used in the method in preventing an infection by a virus, or treating a virus infection, or treating a disease caused by a virus as disclosed herein.

Further, any possible mutation of a virus which may develop, in particular during virus and host contact and interaction, or evolutionary alteration of a virus are also examples which can be used in the method in preventing an infection by a virus, or treating a virus infection, or treating a disease caused by a virus as disclosed herein.

Preferably, in some embodiments the present invention also relates to a method of preventing an infection by a bacterium by the use of a composition, wherein the composition is any composition as defined herein.

Preferably, in some embodiments the present invention also relates to a method of treating a bacterial infection by the use of a composition, wherein the composition is any composition as defined herein.

Preferably, in some embodiments the present invention also relates to a method of treating a disease caused by a bacterial infection by the use of a composition, wherein the composition is any composition as defined herein.

In any one of the methods, the bacterium may be any bacterium defined herein, preferably a respiratory bacterium. In any one of the methods, the disease may be any disease caused by a bacterial infection defined herein.

Preferably, in some embodiments the present invention also relates to a method of preventing an infection by a protozoan by the use of a composition, wherein the composition is any composition as defined herein.

Preferably, in some embodiments the present invention also relates to a method of treating a protozoan infection by the use of a composition, wherein the composition is any composition as defined herein.

Preferably, in some embodiments, the present invention also relates to a method of treating a disease caused by a protozoan infection by the use of a composition, wherein the composition is any composition as defined herein.

In any one of the methods, the protozoan may be any protozoan defined herein. In any one of the methods, the disease may be any disease caused by a protozoan infection defined herein.

Preferably, in some embodiments the present invention also relates to a method of preventing an infection by a fungus by the use of a composition, wherein the composition is any composition as defined herein.

Preferably, in some embodiments the present invention also relates to a method of treating a mycotic infection by the use of a composition, wherein the composition is any composition as defined herein.

Preferably, in some embodiments, the present invention also relates to a method of treating a disease caused by a mycotic infection by the use of a composition, wherein the composition is any composition as defined herein.

In any one of the methods, the fungus may be any fungus defined herein. In any one of the methods, the disease may be any disease caused by a mycotic infection defined herein.

Preferably, the method comprises contacting a bodily fluid with the composition. In particular, the bodily fluid is saliva or a mucous secretion. The mucous secretion may be a nasal secretion or a bronchial secretion. Also preferably, the method may comprise contacting a mucosa with the composition. The mucosa may be mucosa of the nose. The mucosa may be mucosa of the pharynx. The mucosa may be mucosa of the mouth. The mucosa may be mucosa of the bronchia.

### Chewing Gum

The present invention also relates to a chewing gum comprising a compound selected from the group consisting of chitosan or a salt thereof, galactose, mannose, caffeine, and any combination thereof.

Accordingly, in the chewing gum the compound may be caffeine. The compound may be mannose. The compound may be galactose. Preferably, the compound is chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein in accordance with the invention is any combination of the foregoing compounds. The salt may be in pharmaceutically acceptable quality.

In some embodiments, when the compound is chitosan, the chewing gum may further comprise an acid. Accordingly, the chewing gum may comprise chitosan and an acid. Preferably, the acid is selected from the group consisting of, but not limited to, citric acid, acetic acid, ascorbic acid, malic acid, tartaric acid, any fruit acid, a food acidulant, and any combination thereof. More preferably, the acid is citric acid. When chewing, the acid lowers the pH so that at least part of the chitosan is converted into its cationic form. Accordingly, solubility of the chitosan in water is increased.

The chewing gum may comprise a particle. Preferably, the particle is a microparticle or a nanoparticle, more preferably a microparticle. Preferably, the particle, e.g. a microparticle or a nanoparticle, more preferably a microparticle, comprises chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is a particle, preferably a microparticle or a nanoparticle, more preferably a microparticle comprising any combination of the foregoing compounds. The salt may be in pharmaceutically acceptable quality.

In addition to the chitosan or salt thereof, the particle, preferably a microparticle or a nanoparticle, more preferably a microparticle may comprise a further polymer or biopolymer. The polymer or biopolymer can include, but is not limited to, one or more of the following substances or classes of substances: proteins, polysaccharides, hydrocarbons, nucleic acids, aptamers, collagen, collagen-n-hydroxysuccinimide, fibrin, gelatin, albumin, alginates, blood plasma proteins, milk proteins, casein, protein-based polymers, hyaluronic acid, pectins, gummi arabicum and other gums, casein, whey proteins, gluten, starch, cellulose, synthetic polymers for pharmaceutical or cosmetic applications, like polylactic acid, polyglycolic acid, cell lysates of plants and microorganisms, copolymers and/or derivatives and/or mixtures and/or chemical modifications of said polymers and any combination thereof, with different material parameters such as chain length or molecular weight. The polymers may be utilized, for example, as excipients (e.g., for the incorporation and processing of active ingredients), as basic formulation substances (e.g., for cosmetics). Preferably, the further biopolymer is hyaluronic acid. Accordingly, in a preferred embodiment, the particle, preferably a microparticle or a nanoparticle, more preferably a microparticle, further to chitosan or a salt thereof, comprises hyaluronic acid.

In some embodiments, the chewing gum may comprise a co-agglomerate of particles. Preferably, the co-agglomerate is a co-agglomerate of microparticles or a co-agglomerate of nanoparticles; more preferably, the co-agglomerate is a co-agglomerate of microparticles. Preferably, the co-agglomerate of particles, preferably a co-agglomerate of microparticles or a co-agglomerate of nanoparticles, more preferably a co-agglomerate of microparticles, comprises chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is a co-agglomerate of particles, preferably a co-agglomerate of microparticles or a co-agglomerate of nanoparticles, more preferably a co-agglomerate of microparticles, comprising any combination of the foregoing compounds. The salt may be in pharmaceutically acceptable quality.

In addition to the chitosan or salt thereof, the co-agglomerate of particles, preferably a co-agglomerate of microparticles or a co-agglomerate of nanoparticles, more preferably a co-agglomerate of microparticles, may comprise a further polymer or biopolymer. The polymer or biopolymer can include, but is not limited to, one or more of the following substances or classes of substances: proteins, polysaccharides, hydrocarbons, nucleic acids, aptamers, collagen, collagen-n-hydroxysuccinimide, fibrin, gelatin, albumin, alginates, blood plasma proteins, milk proteins, casein, protein-based polymers, hyaluronic acid, pectins, gummi arabicum and other gums, casein, whey proteins, gluten, starch, cellulose, synthetic polymers for pharmaceutical or cosmetic applications, like polylactic acid, polyglycolic acid, cell lysates of plants and microorganisms, copolymers and/or derivatives and/or mixtures and/or chemical modifications of said polymers and any combination thereof, with different material parameters such as chain length or molecular weight. The polymers may be utilized, for example, as excipients (e.g., for the incorporation and processing of active ingredients), as basic formulation substances (e.g., for cosmetics). Preferably, the further biopolymer is hyaluronic acid. Accordingly, in a preferred embodiment, the co-agglomerate of particles, preferably a co-agglomerate of microparticles or nanoparticles, more preferably a co-agglomerate of microparticles, further to chitosan or a salt thereof, comprises hyaluronic acid.

In general, any chitosan or salt thereof, as described herein, can be used in the chewing gum. For example, chitosan from animals or fungi may be used. Preferably, a vegan chitosan (e.g. chitosan from fungi) may be used.

In the chewing gum, the chitosan or the salt thereof may have a degree of deacetylation of 60% or more. The chitosan or the salt thereof may have a degree of deacetylation of 70% or more. The chitosan or the salt thereof may have a degree of deacetylation of 80% or more. The chitosan or the salt thereof may have a degree of deacetylation of 90% or more. The chitosan or the salt thereof may have a degree of deacetylation of 95% or more.

In the chewing gum, the chitosan or the salt thereof may have a degree of deacetylation of from 30% to 60%. the chitosan or the salt thereof may have a degree of deacetylation of from 30% to 60%. The chitosan or the salt thereof may have a degree of deacetylation of from 35% to 55%. The chitosan or the salt thereof may have a degree of deacetylation of from 40% to 50%.

In some embodiments, the chitosan or salt thereof has a molecular weight of from 20000 Da to 460000 Da.

In some embodiments, the chewing gum may comprise an oligochitosan having a molecular weight of below 20000 Da, or a salt of an oligochitosan having a molecular weight of below 20000 Da.

The chewing gum may comprise the compound, in particular chitosan or a salt thereof, in an amount of 0.1% to 10% by weight of the chewing gum. The chewing gum may comprise the compound, in particular chitosan or a salt thereof, in an amount of 0.2 to 5% by weight of the chewing gum. The chewing gum may comprise the compound, in particular chitosan or a salt thereof, in an amount of 0.3 to 3% by weight of the chewing gum. The chewing gum may comprise the compound, in particular chitosan or a salt thereof, in an amount of 0.4 to 4% by weight of the chewing gum. The indication “by weight of the chewing gum”, whenever used throughout this specification, refers to the total weight of the chewing gum, i.e. 100% by weight of the chewing gum.

In general, the chewing gum comprises a gum base in which the further ingredients, in particular the compound of the invention (chitosan or salt thereof, galactose, mannose and/or caffeine), are comprised. Further ingredients may be, as merely illustrative examples, sugar, sweeteners or flavoring agents. As illustrative example, the chewing gum may comprise gum base in an amount of 5 to 50% by weight of the chewing gum, 8 to 40% by weight of the chewing gum or to 30% by weight of the chewing gum.

The composition of gum base formulations can vary substantially depending on the particular product to be prepared and on the desired masticatory and other sensory characteristics of the final product. However, typical ranges (% by weight) of the ingredients in the gum base matrix are: 5 to 80% by weight elastomeric compounds, 5 to 80% by weight elastomer plasticizers, 0 to 40% by weight of waxes, 5 to 35%) by weight softener, 0 to 50% by weight filler, and 0 to 5%> by weight of miscellaneous ingredients such as antioxidants, colorants, etc. The gum base may comprise about 5 to about 95 percent, by weight, of the chewing gum, more commonly the gum base comprises 10 to about 60 percent, by weight, of the gum.

Elastomers provide the rubbery, cohesive nature to the gum, which varies depending on this ingredient's chemical structure and how it may be compounded with other ingredients. Elastomers suitable for use in the gum base and gum of the present invention may include natural or synthetic types.

Elastomer plasticizers vary the firmness of the gum base. Their specificity on elastomer inter-molecular chain breaking (plasticizing) along with their varying softening points cause varying degrees of finished gum firmness and compatibility when used in base. This may be important when one wants to provide more elastomeric chain exposure to the alkane chains of the waxes. The elastomers (rubbers) employed in the gum base may vary depending upon various factors such as the type of gum base desired, the texture of gum composition desired and the other components used in the composition to make the final chewing gum product. Illustrative examples of suitable polymers in gum bases include both natural and synthetic elastomers. For example, those polymers which are suitable in gum base compositions include, without limitation, natural substances (of vegetable origin) such as chicle gum, natural rubber, crown gum, nispero, rosidinha, jelutong, perillo, niger gutta, tunu, balata, guttapercha, lechi capsi, sorva, gutta kay, and the like, and mixtures thereof. Moreover, methyl vinyl ether-maleic anhydride copolymers may be added. Examples of synthetic elastomers include, without limitation, styrene-butadiene copolymers (SBR), polyisobutylene, isobutylene-isoprene copolymers, polyisoprene, polyethylene, polyvinyl acetate, vinyl acetate-vinyl laureate copolymer and the like, and mixtures thereof.

Natural resins may be used according to embodiments of the invention and may be natural rosin esters, often referred to as ester gums including as examples glycerol esters of partially hydrogenated rosins, glycerol esters of polymerised rosins, glycerol esters of partially dimerized rosins, glycerol esters of tally oil rosins, pentaerythritol esters of partially hydrogenated rosins, methyl esters of rosins, partially hydrogenated methyl esters of rosins, pentaerythritol esters of rosins, synthetic resins such as terpene resins derived from alpha-pinene, beta-pinene, and/or d-limonene, and natural terpene resins. In an embodiment of the invention, the resin comprises terpene resins, e.g. derived from alpha-pinene, beta-pinene, and/or d-limonene, natural terpene resins, glycerol esters of gum rosins, tall oil rosins, wood rosins or other derivatives thereof such as glycerol esters of partially hydrogenated rosins, glycerol esters of polymerized rosins, glycerol esters of partially dimerised rosins, pentaerythritol esters of partially hydrogenated rosins, methyl esters of rosins, partially hydrogenated methyl esters of rosins or pentaerythritol esters of rosins and combinations thereof. Gum bases are typically prepared by adding an amount of the elastomer, elastomer plasticizer and filler, and on occasion a vinyl polymer, to a heated (10° C.-120° C.) sigma blade mixer with a front to rear speed ratio of from about 1.2:1 to about 2:1, the higher ratio typically being used for gum base which requires more rigorous compounding of its elastomers.

In an embodiment of the invention, said chewing gum comprises said gum base and one or more chewing gum ingredients.

In an embodiment, said chewing gum ingredients are selected from the group consisting of bulk sweeteners, flavors, dry-binders, tabletting aids, anti-caking agents, emulsifiers, antioxidants, enhancers, absorption enhancers, buffers, high intensity sweeteners, softeners, colors, or any combination thereof.

In addition to the above water-insoluble gum base composition, the bulk portion comprises a generally water-soluble part comprising a range of chewing gum additives. In the present context, the term “chewing gum additive” is used to designate any component, which in a conventional chewing gum manufacturing process is added to the bulk portion. The major proportion of such conventionally used additives is water soluble, but water-insoluble components, such as e.g. water-insoluble flavoring compounds, can also be included. In the present context, chewing gum additives include bulk sweeteners, high intensity sweeteners, flavoring agents, softeners, emulsifiers, coloring agents, binding agents, acidulants, fillers, antioxidants and other components such as pharmaceutically or biologically active substances, conferring desired properties to the finished chewing gum product.

Suitable bulk sweeteners include both sugar and non-sugar sweetening components. Bulk sweeteners typically constitute from about 5 to about 95% by weight of the chewing gum, more typically about 20 to about 80%) by weight such as 30 to 70% or 30 to 60% by weight of the gum.

Useful sugar sweeteners are saccharide-containing components commonly known in the chewing gum art including, but not limited to, sucrose, dextrose, maltose, dextrins, trehalose, D-tagatose, dried invert sugar, fructose, levulose, galactose, corn syrup solids, and the like, alone or in combination.

Sorbitol can be used as a non-sugar sweetener. Other useful non-sugar sweeteners include, but are not limited to, other sugar alcohols such as mannitol, xylitol, hydrogenated starch hydrolysates, maltitol, isomalt, erythritol, lactitol, inulin and the like, alone or in combination.

High intensity artificial sweetening agents can also be used alone or in combination with the above sweeteners. Preferred high intensity sweeteners include, but are not limited to sucralose, aspartame, salts of acesulfame, alitame, saccharin and its salts, cyclamic acid and its salts, glycyrrhizin, dihydrochalcones, thaumatin, monellin, stevioside and the like, alone or in combination.

Usage level of the artificial sweetener will vary considerably and will depend on factors such as potency of the sweetener, rate of release, desired sweetness of the product, level and type of flavor used and cost considerations. Thus, the active level of artificial sweetener may vary from about 0.001 to about 8% by weight (preferably from about 0.02 to about 8% by weight). When carriers used for encapsulation are included, the usage level of the encapsulated sweetener will be proportionately higher. Combinations of sugar and/or non-sugar sweeteners can be used in the chewing gum composition processed in accordance with the invention. Additionally, the softener may also provide additional sweetness such as with aqueous sugar or alditol solutions.

In an embodiment, the chewing gum comprises one or more chewing gum ingredients selected from the group consisting of bulk sweeteners, flavors, dry-binders, tabletting aids, anti-caking agents, emulsifiers, antioxidants, enhancers, absorption enhancers, buffers, or any combination thereof.

Further useful chewing gum base include antioxidants, e.g. butylated hydroxytoluene (BHT), butyl hydroxyanisol (BHA), propylgallate and tocopherols, and preservatives.

A gum base formulation may, in accordance with embodiments of the present invention, comprise one or more softening agents e.g. sucrose esters, tallow, hydrogenated tallow, hydrogenated and partially hydrogenated vegetable oils, cocoa butter, degreased cocoa powder, glycerol monostearate, glyceryl triacetate, lecithin, mono-, di- and triglycerides, acetylated monoglycerides, lanolin, sodium stearate, potassium stearate, glyceryl lecithin, propylene glycol monostearate, glycerine, fatty acids (e.g. stearic, palmitic, oleic and linoleic acids) and combinations thereof. As used herein the term “softener” designates an ingredient, which softens the gum base or chewing gum composition and encompasses waxes, fats, oils, emulsifiers, surfactants and solubilisers. Softeners are typically used in an amount of 0 to 18% by weight, preferably 0 to 12% by weight of the gum base.

Useful emulsifiers can include, but are not limited to, glyceryl monostearate, propylene glycol monostearate, mono- and diglycerides of edible fatty acids, lactic acid esters and acetic acid esters of mono- and diglycerides of edible fatty acids, acetylated mono and diglycerides, sugar esters of edible fatty acids, Na-, K-, Mg- and Ca-stearates, lecithin, hydroxylated lecithin and the like and mixtures thereof are examples of conventionally used emulsifiers which can be added to the chewing gum base. In case of the presence of a biologically or pharmaceutically active ingredient as defined below, the formulation may comprise certain specific emulsifiers and/or solubilisers in order to disperse and release the active ingredient.

Waxes and fats are conventionally used for the adjustment of the texture and for softening of the chewing gum base when preparing chewing gum bases. In connection with the present invention, any conventionally used and suitable type of natural and synthetic wax and fat may be used, such as for instance rice bran wax, polyethylene wax, petroleum wax (refined paraffin and microcrystalline wax), sorbitan monostearate, tallow, propylene glycol, paraffin, beeswax, carnauba wax, candelilla wax, cocoa butter, degreased cocoa powder and any suitable oil or fat, as e.g. completely or partially hydrogenated vegetable oils or completely or partially hydrogenated animal fats.

A chewing gum base may, if desired, include one or more fillers/texturisers including as examples, magnesium and calcium carbonate, sodium sulphate, ground limestone, silicate compounds such as magnesium and aluminum silicate, kaolin and clay, aluminum oxide, silicium oxide, talc, titanium oxide, mono-, di- and tri-calcium phosphates, cellulose polymers, such as wood, and combinations thereof. In an embodiment of the invention, said chewing gum comprises a biodegradable gum base polymer.

The chewing gum may further comprise at least one delivery vehicle and/or excipient selected from the group consisting of lactose anhydrous or monohydrate, povidone, microcystalline cellulose, hydroxypropylcellulose, sodium croscaramellose, magnesium stearate, E171, E172, mannitol, sodium laurylsulphate, ipromellose, methacrylic acid copolymer, macrogol, magnesium stearate, gelatine, saccharose, starch, sorbitol, flavours, sodium saccharine, colloidal silica, titanium dioxide, maltitol syrup, gum arabic, glycerol, aspartame, hydrogenated vegetal oil, sorbitol, citric acid, pectin, caramel, sucrose and methylcellulose.

The chewing gum may comprise flavorings.

The chewing gum may comprise a thickener, e.g. gum arabic.

The chewing gum may comprise a humectant, e.g. glycerol.

As sweeteners, the chewing gum may comprise sorbitols, isomalt, maltitols, xylitol, acesulfame K, and/or sucralose.

The chewing gum may comprise further ingredients, e.g. zinc or a salt of zinc. Accordingly, the chewing gum may comprise zinc. The chewing gum may comprise a salt of zinc, e.g. zinc acetate.

In a preferred embodiment, the chewing gum is sugar-free. In general, a sugar-free gum is made with polyalcohols, usually called polyols, replacing the sugars used in standard gum formulation; these are, for example, sorbitol, mannitol, maltitol, isomalt, lactitol and xylitol. Usually, a sugar-free gum does not comprise glucose syrup. A typical, merely illustrative, formulation of a sugar-free chewing gum, which does not yet include the amounts of the compound of the invention (chitosan or salt thereof, galacatose, mannose, and/or caffeine) may comprise gum base (24.00% by weight of the weight of the chewing gum), mannitol (0 to 5.00% by weight of the chewing gum), maltitol syrup (for example, 85% by weight solids) (7.00 to 12.00% by weight of the chewing gum), sorbitol solution (for example, 70% by weight) (0.00 to 10.00% by weight of the chewing gum), glycerine (1.00 to 6.00% by weight of the chewing gum), xylitol powder (0.00 to 10.00% by weight of the chewing gum), flavor (1.00 to 1.50% by weight of the chewing gum), encapsulated flavors (0.20 to 2.00% by weight of the chewing gum), fruit acid (optional) (1.00% by weight of the chewing gum), color (0.03% by weight of the chewing gum), high-intensity sweeteners (0.20 to 0.40% by weight of the chewing gum), and sorbitol powder (up to 100.00% by weight of the chewing gum).

The chewing gum may be provided with an outer coating (also called glazing) selected from the group consisting of hard coating, soft coating and edible film-coating or any combination thereof. For example, the outer coating may be or may comprise carnauba wax. Accordingly, carnauba way may be used as glazing agent for the chewing gum.

The chewing gum may have the form of a tablet, a dragee (e.g. a pillow shape dragee), a cube, a round gum, or a stick (e.g. a ministick). For example, the chewing gum may be a tablet, or a dragee.

In a particularly preferred embodiment, the chewing gum comprises sorbitols, isomalt, maltitols, xylitol, acesulfame K, sucralose, gum base, flavourings, glycerol, gum Arabic, chitosan, zinc acetate, and carnauba wax (glazing agent). Optionally, the chewing gum may further comprise citric acid.

The chewing gum may be prepared by any method of preparing a chewing gum known to a person skilled in the art, applying, as mere examples and non-limiting, mixing, kneading and/or melting. For example, the chewing gum can be batch mixed, or the chewing gum can be extruded. As a merely illustrative example, first, gum base may be prepared through a melting and straining or filtering process. Next, other ingredients such as nutritive and non-nutritive sweeteners and flavors may be added to the gum base until the warm mixture thickens like dough. The gum base mixture can be heated during this mixing process in order to increase the entropy of the polymers to achieve a more uniform dispersion of ingredients. Then, extrusion technology may be implemented to smooth, form, and shape the gum. Next, the gum may go through a shaping process. For example, cut and wrap (chunk or cube) pieces are severed straight out of the extruder using a vertical cutter. Sheeting is a technique often used for stick, slab and tab gums. Next, gum may be either conditioned by being sprinkled with a powdered polyol or coated via the application of subsequent layers of coating using temperature-controlled coating basins before it is sent to packaging.

The chewing gum may be for any one of the uses described herein, or may be used in any one of the uses or methods described herein. Accordingly, the chewing gum may be for use in preventing an infection by a virus or bacterium, in particular preventing an infection with SARS-CoV-2. Also, the chewing gum may be for use in treating a virus or bacterial infection, e.g. with SARS-CoV-2, and/or may be for use in treating a disease caused by a virus or bacterial infection, e.g. COVID-19.

### Edible Composition

The present invention also relates to an edible composition comprising a compound selected from the group consisting of chitosan or a salt thereof. Accordingly, the compound may be chitosan or a salt of chitosan. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is any combination of the foregoing compounds. The salt may be in pharmaceutically acceptable quality.

When the compound is chitosan, optionally the edible composition may further comprise an acid. Preferably, the acid is selected from the group consisting of, but not limited to, citric acid, acetic acid, ascorbic acid, malic acid, tartaric acid, any fruit acid, a food acidulant, and any combination thereof. More preferably, the acid is citric acid. The acid may lower the pH so that at least part of the chitosan is converted into its cationic form. Accordingly, solubility of the chitosan in water can be increased.

The edible composition may be a dietary supplement. The edible composition may be a food. The edible composition may be a beverage.

Preferably, the edible composition is selected from the group consisting of a candy, a bonbon, a fruit gum, a chocolate, a chewing gum, and a composition for preparing a beverage (e.g. a coffee composition, a tea composition, a sherbet powder, an effervescent tablet). Accordingly, the edible composition may be a candy. The edible composition may be a bonbon. The edible composition may be a fruit gum. The edible composition may be a chocolate. The edible composition may be a chewing gum. The edible composition may be a composition for preparing a beverage. The edible composition may be a coffee composition. The edible composition may be a tea composition. The edible composition may be a sherbet powder. The edible composition may be an effervescent tablet.

Preferably, the edible composition is a beverage selected from the group consisting of tea, coffee, juice, lemonade, milk, cola, a drinkable milk product (e.g. drinkable yoghurt), a beverage obtainable by dissolving a sherbet powder or an effervescent tablet, and an alcoholic beverage. Accordingly, the edible composition is tea. The edible composition is coffee. The edible composition is juice. The edible composition is lemonade. The edible composition is milk. The edible composition is cola. The edible composition is a drinkable milk product. The edible composition is drinkable yoghurt. The edible composition is a beverage obtainable by dissolving a sherbet powder or an effervescent tablet. The edible composition is an alcoholic beverage, e.g. beer or wine.

The edible composition may comprise a particle. Preferably, the particle is a microparticle or a nanoparticle, more preferably the particle is a microparticle. Preferably, the particle, preferably a microparticle or a nanoparticle, more preferably a microparticle, comprises chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is a particle, preferably a microparticle or a nanoparticle, more preferably a microparticle, comprising any combination of the foregoing compounds. The salt may be in pharmaceutically acceptable quality.

In addition to the chitosan or salt thereof, the particle, preferably a microparticle or a nanoparticle, more preferably a microparticle, may comprise a further polymer or biopolymer. The polymer or biopolymer can include, but is not limited to, one or more of the following substances or classes of substances: proteins, polysaccharides, hydrocarbons, nucleic acids, aptamers, collagen, collagen-n-hydroxysuccinimide, fibrin, gelatin, albumin, alginates, blood plasma proteins, milk proteins, casein, protein-based polymers, hyaluronic acid, pectins, gummi arabicum and other gums, casein, whey proteins, gluten, starch, cellulose, synthetic polymers for pharmaceutical or cosmetic applications, like polylactic acid, polyglycolic acid, cell lysates of plants and microorganisms, copolymers and/or derivatives and/or mixtures and/or chemical modifications of said polymers and any combination thereof, with different material parameters such as chain length or molecular weight. The polymers may be utilized, for example, as excipients (e.g., for the incorporation and processing of active ingredients), as basic formulation substances (e.g., for cosmetics). Preferably, the further biopolymer is hyaluronic acid. Accordingly, in a preferred embodiment, the particle, preferably a microparticle or a nanoparticle, more preferably a microparticle, further to chitosan or a salt thereof, comprises hyaluronic acid.

In some embodiments, the edible composition may comprise a co-agglomerate of particles, Preferably, the co-agglomerate is a co-agglomerate of microparticles or a co-agglomerate of nanoparticles, more preferably the co-agglomerate is a co-agglomerate of microparticles. Preferably, the co-agglomerate of particles, preferably a co-agglomerate of microparticles or a co-agglomerate of nanoparticles, more preferably a co-agglomerate of microparticles, comprises chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is a co-agglomerate of particles, preferably a co-agglomerate of microparticles or a co-agglomerate of nanoparticles, more preferably a co-agglomerate of microparticles comprising any combination of the foregoing compounds. The salt may be in pharmaceutically acceptable quality.

In addition to the chitosan or salt thereof, the co-agglomerate of particles, preferably a co-agglomerate of microparticles or a co-agglomerate of nanoparticles, more preferably a co-agglomerate of microparticles, may comprise a further polymer or biopolymer. The polymer or biopolymer can include, but is not limited to, one or more of the following substances or classes of substances: proteins, polysaccharides, hydrocarbons, nucleic acids, aptamers, collagen, collagen-n-hydroxysuccinimide, fibrin, gelatin, albumin, alginates, blood plasma proteins, milk proteins, casein, protein-based polymers, hyaluronic acid, pectins, gummi arabicum and other gums, casein, whey proteins, gluten, starch, cellulose, synthetic polymers for pharmaceutical or cosmetic applications, like polylactic acid, polyglycolic acid, cell lysates of plants and microorganisms, copolymers and/or derivatives and/or mixtures and/or chemical modifications of said polymers and any combination thereof, with different material parameters such as chain length or molecular weight. The polymers may be utilized, for example, as excipients (e.g., for the incorporation and processing of active ingredients), as basic formulation substances (e.g., for cosmetics). Preferably, the further biopolymer is hyaluronic acid. Accordingly, in a preferred embodiment, the co-agglomerate of particles, preferably a co-agglomerate of microparticles or a co-agglomerate of nanoparticles, more preferably a co-agglomerate of microparticles, further to chitosan or a salt thereof, comprises hyaluronic acid.

In the edible composition, the chitosan or the salt thereof may have a degree of deacetylation of 60% or more. The chitosan or the salt thereof may have a degree of deacetylation of 70% or more. The chitosan or the salt thereof may have a degree of deacetylation of 80% or more. The chitosan or the salt thereof may have a degree of deacetylation of 90% or more. The chitosan or the salt thereof may have a degree of deacetylation of 95% or more.

In the edible composition, the chitosan or the salt thereof may have a degree of deacetylation of from 30% to 60%. the chitosan or the salt thereof may have a degree of deacetylation of from 30% to 60%. The chitosan or the salt thereof may have a degree of deacetylation of from 35% to 55%. The chitosan or the salt thereof may have a degree of deacetylation of from 40% to 50%.

In some embodiments, the chitosan or salt thereof has a molecular weight of from 20000 Da to 460000 Da.

In some embodiments, the edible composition may comprise an oligochitosan having a molecular weight of below 20000 Da, or a salt of an oligochitosan having a molecular weight of below 20000 Da.

The edible composition may be for any one of the uses described herein, or may be used in any one of the uses or methods described herein.

### Composition Suitable for Oral, Nasal or Bronchial Administration

The present invention also relates to a composition suitable for oral, nasal or bronchial administration, the composition comprising a particle, wherein the particle comprises chitosan or a salt thereof. Accordingly, the particle comprises chitosan or a salt of chitosan. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is any combination of the foregoing compounds. The salt may be in pharmaceutically acceptable quality.

Preferably, in some embodiments the present invention also relates to a composition suitable for oral, nasal or bronchial administration, the composition comprising a microparticle or a nanoparticle, more preferably a microparticle, wherein the microparticle or a nanoparticle, more preferably a microparticle comprises chitosan or a salt thereof. Accordingly, the microparticle or a nanoparticle, more preferably a microparticle comprises chitosan or a salt of chitosan. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is any combination of the foregoing compounds. The salt may be in pharmaceutically acceptable quality.

When the compound is chitosan, optionally the composition may further comprise an acid. Preferably, the acid is selected from the group consisting of, but not limited to, citric acid, acetic acid, ascorbic acid, malic acid, tartaric acid, any fruit acid, a food acidulant, and any combination thereof. More preferably, the acid is citric acid. The acid may lower the pH so that at least part of the chitosan is converted into its cationic form. Accordingly, solubility of the chitosan in water can be increased.

The composition may be suitable for oral administration.

The composition may be suitable for nasal administration.

The composition may be suitable for bronchial administration.

In addition to the chitosan or salt thereof, the particle, preferably a microparticle or a nanoparticle, more preferably a microparticle may comprise a further polymer or biopolymer. The polymer or biopolymer can include, but is not limited to, one or more of the following substances or classes of substances: proteins, polysaccharides, hydrocarbons, nucleic acids, aptamers, collagen, collagen-n-hydroxysuccinimide, fibrin, gelatin, albumin, alginates, blood plasma proteins, milk proteins, casein, protein-based polymers, hyaluronic acid, pectins, gummi arabicum and other gums, casein, whey proteins, gluten, starch, cellulose, synthetic polymers for pharmaceutical or cosmetic applications, like polylactic acid, polyglycolic acid, cell lysates of plants and microorganisms, copolymers and/or derivatives and/or mixtures and/or chemical modifications of said polymers and any combination thereof, with different material parameters such as chain length or molecular weight. The polymers may be utilized, for example, as excipients (e.g., for the incorporation and processing of active ingredients), as basic formulation substances (e.g., for cosmetics). Preferably, the further biopolymer is hyaluronic acid. Accordingly, in a preferred embodiment, the particle, preferably a microparticle or a nanoparticle, more preferably a microparticle, further to chitosan or a salt thereof, comprises hyaluronic acid.

In some embodiments, the edible composition may comprise a co-agglomerate of particles. Preferably, the co-agglomerate is a co-agglomerate of microparticles or a co-agglomerate of nanoparticles; more preferably, the co-agglomerate is a co-agglomerate of microparticles. Preferably, the co-agglomerate of particles, preferably a co-agglomerate of microparticles or a co-agglomerate of nanoparticles, more preferably a co-agglomerate of microparticles, comprises chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is a co-agglomerate of particles, preferably a co-agglomerate of microparticles or a co-agglomerate of nanoparticles, more preferably a co-agglomerate of microparticles, comprising any combination of the foregoing compounds. The salt may be in pharmaceutically acceptable quality.

In addition to the chitosan or salt thereof, the co-agglomerate of particles, preferably a co-agglomerate of microparticles or a co-agglomerate of nanoparticles, more preferably a co-agglomerate of microparticles, may comprise a further polymer or biopolymer. The polymer or biopolymer can include, but is not limited to, one or more of the following substances or classes of substances: proteins, polysaccharides, hydrocarbons, nucleic acids, aptamers, collagen, collagen-n-hydroxysuccinimide, fibrin, gelatin, albumin, alginates, blood plasma proteins, milk proteins, casein, protein-based polymers, hyaluronic acid, pectins, gummi arabicum and other gums, casein, whey proteins, gluten, starch, cellulose, synthetic polymers for pharmaceutical or cosmetic applications, like polylactic acid, polyglycolic acid, cell lysates of plants and microorganisms, copolymers and/or derivatives and/or mixtures and/or chemical modifications of said polymers and any combination thereof, with different material parameters such as chain length or molecular weight. The polymers may be utilized, for example, as excipients (e.g., for the incorporation and processing of active ingredients), as basic formulation substances (e.g., for cosmetics). Preferably, the further biopolymer is hyaluronic acid. Accordingly, in a preferred embodiment, the co-agglomerate of particles, more preferably a co-agglomerate of microparticles or a co-agglomerate of nanoparticles, more preferably a co-agglomerate of microparticles, further to chitosan or a salt thereof, comprises hyaluronic acid.

In the composition, the chitosan or the salt thereof may have a degree of deacetylation of 60% or more. The chitosan or the salt thereof may have a degree of deacetylation of 70% or more.

The chitosan or the salt thereof may have a degree of deacetylation of 80% or more. The chitosan or the salt thereof may have a degree of deacetylation of 90% or more. The chitosan or the salt thereof may have a degree of deacetylation of 95% or more.

In the composition, the chitosan or the salt thereof may have a degree of deacetylation of from 30% to 60%. the chitosan or the salt thereof may have a degree of deacetylation of from 30% to 60%. The chitosan or the salt thereof may have a degree of deacetylation of from 35% to 55%. The chitosan or the salt thereof may have a degree of deacetylation of from 40% to 50%.

In some embodiments, the chitosan or salt thereof has a molecular weight of from 20000 Da to 460000 Da.

In some embodiments, the composition may comprise an oligochitosan having a molecular weight of below 20000 Da, or a salt of an oligochitosan having a molecular weight of below 20000 Da.

The composition may be a solid composition.

The composition may be a liquid composition.

Preferably, the composition is selected from the group consisting of a composition for spraying or gargling, a mouth rinse, a gel for application to the oral cavity, a nose spray, a nose oil, a nose drop formulation, and a nasal irrigation formulation. Accordingly, the composition may be a composition for spraying or gargling. The composition may be a mouth rinse. The composition may be a gel for application to the oral cavity. The composition may be a nose spray. The composition may be a nose oil. The composition may be a nose drop formulation. The composition may be a nasal irrigation formulation.

The composition suitable for oral, nasal or bronchial administration may be for any one of the uses described herein, or may be used in any one of the uses or methods described herein.

### Sustained Delivery System

The present invention also relates to a sustained delivery system comprising chitosan or a salt thereof. Accordingly, the sustained delivery system comprises chitosan or a salt of chitosan. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is any combination of the foregoing compounds. The salt may be in pharmaceutically acceptable quality.

When the compound is chitosan, optionally the sustained delivery system may further comprise an acid. Preferably, the acid is selected from the group consisting of, but not limited to, citric acid, acetic acid, ascorbic acid, malic acid, tartaric acid, any fruit acid, a food acidulant, and any combination thereof. More preferably, the acid is citric acid. The acid may lower the pH so that at least part of the chitosan is converted into its cationic form. Accordingly, solubility of the chitosan in water can be increased.

Preferably, the sustained delivery system an oral or buccal sustained delivery system.

Preferably, the sustained delivery system comprises a solid or semi-solid dosage form.

The sustained delivery system may be selected from the group consisting of a film, a layered structure of films, a tablet, a capsule, a gel body (e.g. a gel for oral cavity), a chewing gum, a candy, a bonbon, a fruit gum, and a chocolate. Accordingly, the sustained delivery system ma be a film. The sustained delivery system may be a layered structure of films. The sustained delivery system may be a tablet. The sustained delivery system may be a capsule. The sustained delivery system may be a gel body. The sustained delivery system may be a gel for oral cavity. The sustained delivery system may be a chewing gum. The sustained delivery system may be a candy. The sustained delivery system may be a bonbon. The sustained delivery system may be a fruit gum. The sustained delivery system may be a chocolate.

The sustained delivery system may comprise a particle, preferably a microparticle or nanoparticle, more preferably a microparticle, wherein the particle, preferably the microparticle or nanoparticle, more preferably a microparticle comprises chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is a particle, preferably a microparticle or nanoparticle, more preferably a microparticle, comprising any combination of the foregoing compounds. The salt may be in pharmaceutically acceptable quality.

In addition to the chitosan or salt thereof, the particle, preferably a microparticle or nanoparticle, more preferably a microparticle, may comprise a further polymer or biopolymer. The polymer or biopolymer can include, but is not limited to, one or more of the following substances or classes of substances: proteins, polysaccharides, hydrocarbons, nucleic acids, aptamers, collagen, collagen-n-hydroxysuccinimide, fibrin, gelatin, albumin, alginates, blood plasma proteins, milk proteins, casein, protein-based polymers, hyaluronic acid, pectins, gummi arabicum and other gums, casein, whey proteins, gluten, starch, cellulose, synthetic polymers for pharmaceutical or cosmetic applications, like polylactic acid, polyglycolic acid, cell lysates of plants and microorganisms, copolymers and/or derivatives and/or mixtures and/or chemical modifications of said polymers and any combination thereof, with different material parameters such as chain length or molecular weight. The polymers may be utilized, for example, as excipients (e.g., for the incorporation and processing of active ingredients), as basic formulation substances (e.g., for cosmetics). Preferably, the further biopolymer is hyaluronic acid. Accordingly, in a preferred embodiment, the particle, preferably a microparticle or nanoparticle, more preferably a microparticle, further to chitosan or a salt thereof, comprises hyaluronic acid.

In some embodiments, the sustained delivery system may comprise a co-agglomerate of particles. Preferably the co-agglomerate is a co-agglomerate of microparticles or a co-agglomerate of nanoparticles; more preferably, the co-agglomerate is a co-agglomerate of microparticles. Preferably, the co-agglomerate of microparticles comprises chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is a co-agglomerate particles, preferably a co-agglomerate of microparticles or a co-agglomerate of nanoparticles, more preferably a co-agglomerate of microparticles, comprising any combination of the foregoing compounds. The salt may be in pharmaceutically acceptable quality.

In addition to the chitosan or salt thereof, the co-agglomerate of particles, preferably a co-agglomerate of microparticles or a co-agglomerate of nanoparticles, more preferably a co-agglomerate of microparticles, may comprise a further polymer or biopolymer. The polymer or biopolymer can include, but is not limited to, one or more of the following substances or classes of substances: proteins, polysaccharides, hydrocarbons, nucleic acids, aptamers, collagen, collagen-n-hydroxysuccinimide, fibrin, gelatin, albumin, alginates, blood plasma proteins, milk proteins, casein, protein-based polymers, hyaluronic acid, pectins, gummi arabicum and other gums, casein, whey proteins, gluten, starch, cellulose, synthetic polymers for pharmaceutical or cosmetic applications, like polylactic acid, polyglycolic acid, cell lysates of plants and microorganisms, copolymers and/or derivatives and/or mixtures and/or chemical modifications of said polymers and any combination thereof, with different material parameters such as chain length or molecular weight. The polymers may be utilized, for example, as excipients (e.g., for the incorporation and processing of active ingredients), as basic formulation substances (e.g., for cosmetics). Preferably, the further biopolymer is hyaluronic acid. Accordingly, in a preferred embodiment, the co-agglomerate of or a co-agglomerate of nanoparticles, more preferably a co-agglomerate of microparticles, microparticles, further to chitosan or a salt thereof, comprises hyaluronic acid.

In the sustained delivery system, the chitosan or the salt thereof may have a degree of deacetylation of 60% or more. The chitosan or the salt thereof may have a degree of deacetylation of 70% or more. The chitosan or the salt thereof may have a degree of deacetylation of 80% or more. The chitosan or the salt thereof may have a degree of deacetylation of 90% or more. The chitosan or the salt thereof may have a degree of deacetylation of 95% or more.

In the sustained delivery system, the chitosan or the salt thereof may have a degree of deacetylation of from 30% to 60%. the chitosan or the salt thereof may have a degree of deacetylation of from 30% to 60%. The chitosan or the salt thereof may have a degree of deacetylation of from 35% to 55%. The chitosan or the salt thereof may have a degree of deacetylation of from 40% to 50%.

In some embodiments, the chitosan or salt thereof has a molecular weight of from 20000 Da to 460000 Da.

In some embodiments, the sustained delivery system may comprise an oligochitosan having a molecular weight of below 20000 Da, or a salt of an oligochitosan having a molecular weight of below 20000 Da.

The sustained delivery system may be for any one of the uses described herein, or may be used in any one of the uses or methods described herein.

### Composition Suitable for Treating a Surface or Skin, Method of Treating Surface or Skin, and Use Thereof

The present invention also relates to a composition suitable for treating a surface or skin, the composition comprising a microparticle, wherein the microparticle comprises chitosan or a salt thereof. Accordingly, the microparticle comprises chitosan or a salt of chitosan. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is any combination of the foregoing compounds. The salt may be in pharmaceutically acceptable quality.

When the compound is chitosan, optionally the composition may further comprise an acid. Preferably, the acid is selected from the group consisting of, but not limited to, citric acid, acetic acid, ascorbic acid, malic acid, tartaric acid, any fruit acid, a food acidulant, and any combination thereof. More preferably, the acid is citric acid. The acid may lower the pH so that at least part of the chitosan is converted into its cationic form. Accordingly, solubility of the chitosan in water can be increased.

In addition to the chitosan or salt thereof, the microparticle may comprise a further polymer or biopolymer. The polymer or biopolymer can include, but is not limited to, one or more of the following substances or classes of substances: proteins, polysaccharides, hydrocarbons, nucleic acids, aptamers, collagen, collagen-n-hydroxysuccinimide, fibrin, gelatin, albumin, alginates, blood plasma proteins, milk proteins, casein, protein-based polymers, hyaluronic acid, pectins, gummi arabicum and other gums, casein, whey proteins, gluten, starch, cellulose, synthetic polymers for pharmaceutical or cosmetic applications, like polylactic acid, polyglycolic acid, cell lysates of plants and microorganisms, copolymers and/or derivatives and/or mixtures and/or chemical modifications of said polymers and any combination thereof, with different material parameters such as chain length or molecular weight. The polymers may be utilized, for example, as excipients (e.g., for the incorporation and processing of active ingredients), as basic formulation substances (e.g., for cosmetics). Preferably, the further biopolymer is hyaluronic acid. Accordingly, in a preferred embodiment, the microparticle, further to chitosan or a salt thereof, comprises hyaluronic acid.

In some embodiments, the composition may comprise a co-agglomerate of microparticles. Preferably, the co-agglomerate of microparticles comprises chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is a co-agglomerate of microparticles comprising any combination of the foregoing compounds. The salt may be in pharmaceutically acceptable quality.

In addition to the chitosan or salt thereof, the co-agglomerate of microparticles may comprise a further polymer or biopolymer. The polymer or biopolymer can include, but is not limited to, one or more of the following substances or classes of substances: proteins, polysaccharides, hydrocarbons, nucleic acids, aptamers, collagen, collagen-n-hydroxysuccinimide, fibrin, gelatin, albumin, alginates, blood plasma proteins, milk proteins, casein, protein-based polymers, hyaluronic acid, pectins, gummi arabicum and other gums, casein, whey proteins, gluten, starch, cellulose, synthetic polymers for pharmaceutical or cosmetic applications, like polylactic acid, polyglycolic acid, cell lysates of plants and microorganisms, copolymers and/or derivatives and/or mixtures and/or chemical modifications of said polymers and any combination thereof, with different material parameters such as chain length or molecular weight. The polymers may be utilized, for example, as excipients (e.g., for the incorporation and processing of active ingredients), as basic formulation substances (e.g., for cosmetics). Preferably, the further biopolymer is hyaluronic acid. Accordingly, in a preferred embodiment, the co-agglomerate of microparticles, further to chitosan or a salt thereof, comprises hyaluronic acid.

In the composition, the chitosan or the salt thereof may have a degree of deacetylation of 60% or more. The chitosan or the salt thereof may have a degree of deacetylation of 70% or more. The chitosan or the salt thereof may have a degree of deacetylation of 80% or more. The chitosan or the salt thereof may have a degree of deacetylation of 90% or more. The chitosan or the salt thereof may have a degree of deacetylation of 95% or more.

In the composition, the chitosan or the salt thereof may have a degree of deacetylation of from 30% to 60%. the chitosan or the salt thereof may have a degree of deacetylation of from 30% to 60%. The chitosan or the salt thereof may have a degree of deacetylation of from 35% to 55%. The chitosan or the salt thereof may have a degree of deacetylation of from 40% to 50%.

In some embodiments, the chitosan or salt thereof has a molecular weight of from 20000 Da to 460000 Da.

The composition suitable for treating a surface or skin may be selected from the group consisting of a disinfectant, a skin-caring composition (e.g. a lotion), a soap (e.g. a liquid soap), and a detergent formulation (e.g. a laundry detergent formulation or a dish detergent formulation). Accordingly, the composition suitable for treating a surface or skin may be a disinfectant. The composition may be a skin-caring composition. The composition may be a lotion. The composition may be a soap. The composition may be a liquid soap. The composition may be a detergent formulation. The composition may be a laundry detergent formulation. The composition may be a dish detergent formulation.

The composition suitable for treating a surface or skin may be for any one of the uses described herein, or may be used in any one of the uses or methods described herein.

The present invention also relates to a method of treating a surface or skin, the method comprising contacting the surface or skin with the composition for treating a surface or skin of the invention. Preferably, the method comprises inactivating a virus or bacterium. The virus or bacterium may be any virus or bacterium defined herein.

Preferably, the virus is a respiratory virus. More preferably, the virus is selected from the group consisting of a coronavirus, preferably a human-pathogenic coronavirus, an influenza virus, a rhinovirus, and a respiratory syncytial virus [RSV]. Even more preferably, the human-pathogenic coronavirus is a betacoronavirus. The human-pathogenic coronavirus may be SARS-CoV-2. The human-pathogenic coronavirus may be MERS-CoV. The human-pathogenic coronavirus may be SARS-CoV-1. Most preferably, the human-pathogenic coronavirus is SARS-CoV-2.

The present invention also relates to a use of a composition for treating a surface or skin of the invention for inactivating a virus or bacterium. In particular, the use comprises contacting the surface or skin with the composition for treating a surface or skin of the invention.

The virus or bacterium may be any virus or bacterium defined herein. Preferably, the virus is a respiratory virus. More preferably, the virus is selected from the group consisting of a coronavirus, preferably a human-pathogenic coronavirus, an influenza virus, a rhinovirus, and a respiratory syncytial virus [RSV]. Even more preferably, the human-pathogenic coronavirus is a betacoronavirus. The human-pathogenic coronavirus may be SARS-CoV-2. The human-pathogenic coronavirus may be MERS-CoV. The human-pathogenic coronavirus may be SARS-CoV-1. Most preferably, the human-pathogenic coronavirus is SARS-CoV-2.

The use may be a non-medical use.

### Method of Increasing the Binding of Virus Neutralizing Antibodies

The present invention also relates to a method of increasing the binding of virus neutralizing antibodies, the method comprising contacting a fluid that contains a virus and virus neutralizing antibodies, wherein the virus neutralizing antibodies bind to said virus, with a compound selected from the group consisting of chitosan or a salt thereof, galactose, mannose, caffeine, and any combination thereof.

Preferably, the fluid is a bodily fluid. For example, the bodily fluid may be selected from the group consisting of blood, saliva and a mucous secretion. Accordingly, the bodily fluid may be saliva. The bodily fluid may be blood. The bodily fluid may be a mucous secretion. The mucous secretion may be a nasal secretion and/or a bronchial secretion. For example, the composition is preferably delivered to saliva or a mucous secretion of the respiratory tract, as respiratory viruses, such as e.g. SARS-CoV-2, after entering the human body are typically present in such bodily fluids. Any method known to a person skilled in the art may be used for contacting the bodily fluid with the compound or combination thereof, as merely illustrative examples spraying, gargling, drinking a beverage, eating an edible composition, chewing a chewing gum, injecting or infusing into a blood vessel, etc.

In addition or alternatively, the method may comprise contacting a mucosa with the compound. In particular, when the virus is a respiratory virus, such as e.g. the virus SARS-CoV-2, the mucosa may be selected from the group consisting of mucosa of the nose, mucosa of the pharynx, mucosa of the mouth, and mucosa of the bronchia. Accordingly, the mucosa may be mucosa of the nose. The mucosa may be mucosa of the pharynx. The mucosa may be mucosa of the mouth. The mucosa may be mucosa of the bronchia.

The method may comprise contacting a skin of surface with the compound. In this regard, pathogens, e.g. viruses and bacteria, may be found on skin or a surface. Accordingly, a skin, e.g. the skin of humans, or any surface can be treated with any compound described herein, for any one of the uses and methods described herein.

The method may comprise contacting a wound or a burn with the compound. In this regard, pathogens, e.g. viruses and bacteria, may be found in a wound or a burn. Accordingly, a wound or a burn, e.g. a wound or a burn of human skin, can be treated with any compound described herein, for any one of the uses and methods described herein.

Exemplary viruses which can be used in the method of increasing the binding of virus neutralizing antibodies may be selected from the group consisting of, but are not limited to, a coronavirus, preferably a human-pathogenic coronavirus, an influenza virus, a parainfluenza virus, a rhinovirus, a respiratory syncytial virus [RSV], an adenovirus, an orthomyxivirus, an Epstein-Barr virus, a herpes virus (e.g. herpes simplex), and a measles virus. Other exemplary viruses may be selected from the group consisting of influenza A, HIV-1; hepatitis A, B, C; HPV; EBV; norovirus; herpes simplex; cytomegalovirus; WSSV; RSV; plant virus like; TMV; AMV; TNV; BYMV; PSV; FMV; PVX; acute bee paralyisi virus; IAPV; chronic bee paralysis virus; varroa vectored virus; and in general any RNA and DNA virus. Infection with any virus or bacterium to which the compound (chitosan or salt thereof, galactose, mannose and/or caffeine) can bind can be prevented and/or treated. Illustrative viruses, bacteria, protozoa, and fungi include viruses and bacteria inducing diseases in humans, animals, bees and/or plants.

Further, any possible subunit of a virus or part of a virus or virion which is able exert an infection are also examples which can be used in the method increasing the binding of virus neutralizing antibodies as disclosed herein.

Further, any possible mutation of a virus which may develop, in particular during virus and host contact and interaction, or evolutionary alteration of a virus are also examples which can be used in the method increasing the binding of virus neutralizing antibodies as disclosed herein.

Preferably, the virus is a respiratory virus.

Preferably, the virus is selected from the group consisting of a coronavirus, more preferably a human-pathogenic coronavirus, an influenza virus, a rhinovirus, and a respiratory syncytial virus [RSV].

Preferably, the virus is a coronavirus. As illustrative examples, the family of coronaviridae comprises an alphacoronavirus, a betacoronavirus, a deltacoronavirus, a gammacoronavirus, or an alphaletovirus. Preferably, the coronavirus is a human-pathogenic coronavirus. More preferably, the human-pathogenic coronavirus is a betacoronavirus. Even more preferably, the human-pathogenic coronavirus is selected from the group consisting of SARS-CoV-2, MERS-CoV and SARS-Cov-1.

Accordingly, the human-pathogenic coronavirus may be SARS-CoV-1. The human-pathogenic coronavirus may be MERS-CoV. Most preferably, the human-pathogenic coronavirus is SARS-CoV-2.

Preferably, the compound increases binding of SARS-CoV-2 neutralizing antibodies to the spike protein of SARS-CoV-2 or parts thereof or the nucleocapsid protein or parts thereof.

Interestingly, the inventors found out that the compound of the present invention increases binding of SARS-CoV-2 neutralizing antibodies to the spike protein of SARS-CoV-2. In particular, binding of neutralizing antibodies to a distinct part of the spike protein sequence is increased. Said part of the spike protein sequence is the RBD domain or part of the RBD domain. A part of a sequence can be understood as the sequence having a different length which length can be shorter. The difference of the sequence of the part of the sequence compared to the complete sequence can be a shorter sequence at the N-terminus or C-terminus or at any length or sequence positions in between the N-terminus or C-terminus. As an example, it could be shown that chitosan increases the binding ability of SARS-CoV-2 neutralizing antibodies to spike protein, in particular to the RBD domain of the spike protein. In addition, or alternatively, it could be also shown that chitosan increases the binding ability of antibodies specific for the nucleocapsid protein of SARS-CoV-2. Without being bound to any theory, the increased binding ability to antibodies specific to SARS-CoV-2, such as antibodies specific for the spike protein, in particular the RBD domain or parts thereof, or antibodies specific for the nucleocapsid or parts thereof may be the result of an stabilizing effect of the spike protein, RBD domain, or the nucleocapsid mediated by the chitosan.

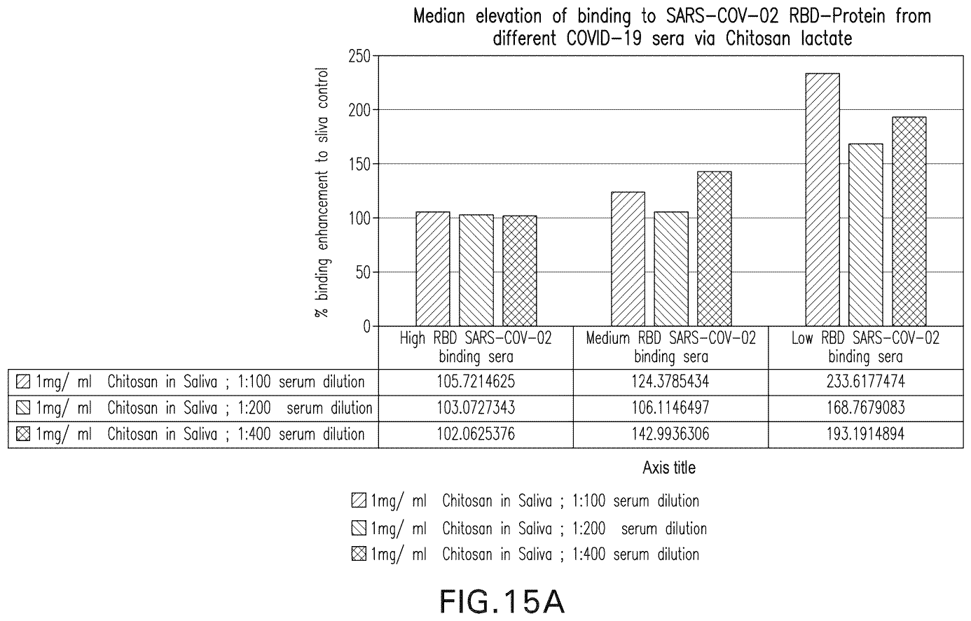

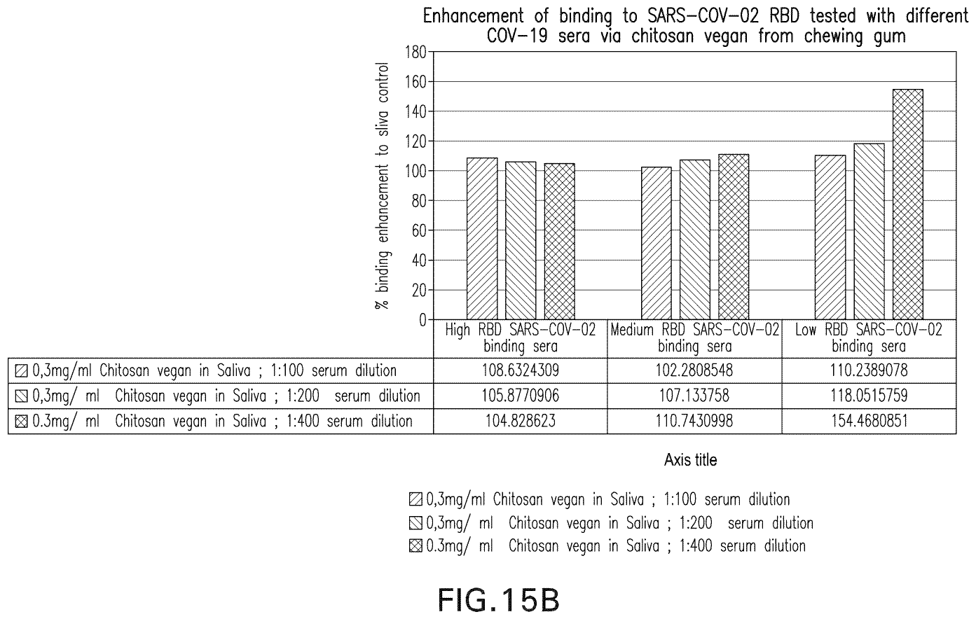

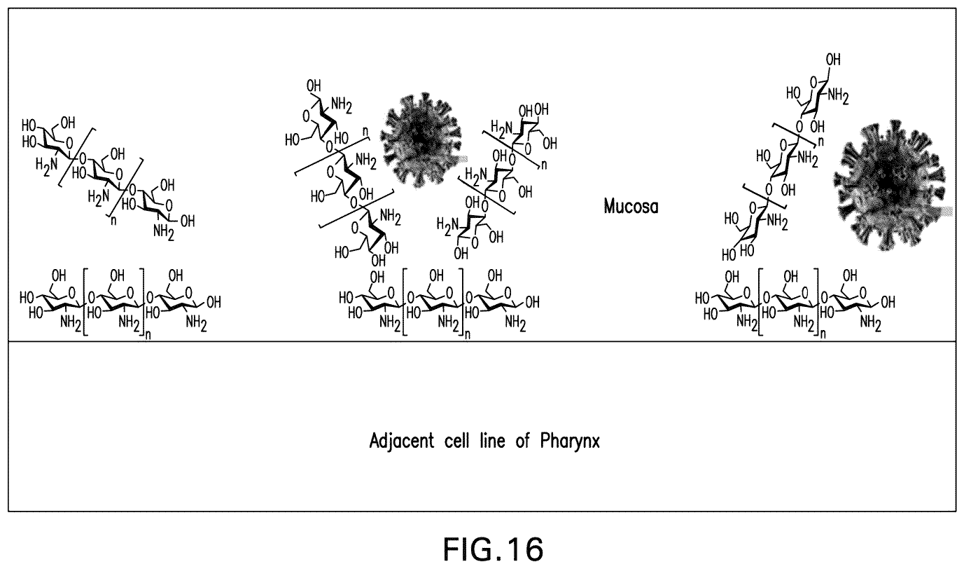

Preferably, the RBD domain or parts thereof or the nucleocapsid or parts thereof is selected from the group consisting of the sequences as shown in SEQ ID NO:1, 2, 3, 4, 5, 6, and 7. The inventors found out that the sequences of SEQ ID NO:1, 2, 3, 4, 5, 6 and 7 are sequences which represent immunogenic structures within the RBD domain of the spike protein or the nucleocapsid. Said sequences are crucial for binding of antibodies, e.g. neutralizing antibodies to the spike protein of SARS-CoV-2 or the nucleocapsid protein of SARS-CoV-2. Advantageously, the compound of the present invention, in particular chitosan is able to increase the binding of SARS-CoV-2 neutralising antibodies to the spike protein. This is shown in e.g. in FIGS. 15A, B.

The method of increasing the binding of virus neutralizing antibodies may be a method of treating a virus infection or treating a disease caused by a virus infection.

The disease caused by a virus infection may be any disease caused by a virus, for example, a disease caused by a coronavirus, preferably a human-pathogenic coronavirus, an influenza virus, a parainfluenza virus, a rhinovirus, a respiratory syncytial virus [RSV], an adenovirus, an orthomyxivirus, an Epstein-Barr virus, a herpes virus (e.g. herpes simplex), and a measles virus.

Preferably, the disease is selected from the group consisting of COVID-19, MERS and SARS.

Accordingly, the disease may be SARS. The disease may be MERS. Most preferably, the disease is COVID-19.

According to the invention, the compound is selected from the group consisting of chitosan or a salt thereof, galactose, mannose, caffeine, and any combination thereof. Accordingly, the compound may be caffeine. The compound may be mannose. The compound may be galactose. Preferably, the compound is chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein in accordance with the invention is any combination of the foregoing compounds. The salt may be in pharmaceutically acceptable quality.

The method of increasing the binding of virus neutralizing antibodies may comprise contacting the fluid that contains the virus and virus neutralizing antibodies with a composition that contains the compound selected from the group consisting of chitosan or a salt thereof, galactose, mannose, caffeine, and any combination thereof. Any one of the compositions described herein can be used in any one of the uses and methods described herein. Accordingly, in particular, any one of the compositions described herein can be used in any one of the uses and methods of increasing the binding of virus-neutralizing antibodies as described herein throughout this specification; preferably wherein the virus is SARS-CoV-2.

Accordingly, in the composition, the compound is selected from the group consisting of chitosan or a salt thereof, galactose, mannose, caffeine, and any combination thereof. In the composition, the compound may be caffeine. In the composition, the compound may be mannose. In the composition, the compound may be galactose. Preferably, in the composition the compound is chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. The salt may be in pharmaceutically acceptable quality. Also described herein in accordance with the invention is any combination of the foregoing compounds in the composition. The salt may be in pharmaceutically acceptable quality.

As an illustrative example, the composition may be a substantially pure preparation of the compound or the combination thereof. Also, the composition may comprise, as illustrative examples, 1% or more, 5% or more, 10 or more, 20% or more, 50% or more, 80% or more, or 90% or more of the compound or the combination thereof.

When the compound is chitosan, optionally the composition may further comprise an acid. Preferably, the acid is selected from the group consisting of, but not limited to, citric acid, acetic acid, ascorbic acid, malic acid, tartaric acid, any fruit acid, a food acidulant, and any combination thereof. More preferably, the acid is citric acid. The acid may lower the pH so that at least part of the chitosan is converted into its cationic form. Accordingly, solubility of the chitosan in water can be increased.

The composition may be a solid composition. As merely illustrative examples, a solid composition may be in the form of a powder, particles, a tablet, a pill, a capsule, etc. The term “solid composition” also includes semi-solid compositions, such as e.g. gels, creams, pastes, ointments, salves, etc.

The composition may be a liquid composition, e.g. a solution.

Preferably, the composition is an edible composition. Any edible composition known to a person skilled in the art may be contemplated, e.g. a food or beverage. In general, as understood by a person skilled in the art, an edible composition is digestible. In the oral cavity, the composition can be contacted with the saliva.

Preferably, the composition may be a composition suitable for oral, nasal or bronchial administration. Any composition known for oral, nasal or bronchial administration may be contemplated. Accordingly, the composition may be a composition for intranasal or inhaled administration, e.g. local administration to the respiratory tract and/or the bronchia. Also, as illustrative examples, the composition may be a spray or solution for gargling.

Also, the composition may be a pharmaceutical composition. The pharmaceutical composition comprises the compound or combination thereof and may optionally further comprise a pharmaceutically acceptable carrier, excipient or stabilizer, such as those described in Remington's Pharmaceutical Sciences 16^{th} edition, Osol, A. Ed. (1980) or 23^{rd} edition (2020), provided that they do not adversely affect the desired characteristics of the formulation. As used herein, “pharmaceutically acceptable carrier” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, asparagine, 2-phenylalanine, and threonine; sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as glutathione, thioctic acid, sodium thioglycolate, thioglycerol, [alpha]-monothioglycerol, and sodium thio sulfate, low molecular weight proteins, such as human serum albumin, bovine serum albumin, gelatin, or other immunoglobulins; and hydrophilic polymers, such as polyvinylpyrrolidone.

Preferably, the composition is a dietary supplement. In a general, a dietary supplement refers to a manufactured product intended to supplement one's diet. For example, the dietary supplement may be in form of a pill, capsule, tablet, powder or liquid. The dietary supplement may be a chewing gum. Preferably, in particular when the composition is solid, e.g. in form of a powder, the dietary supplement is suitable for being added to a beverage. More preferably, the beverage is selected from the group consisting of tea, coffee, juice, lemonade, milk, cola, a drinkable milk product (e.g. drinkable yoghurt), a beverage obtainable by dissolving a sherbet powder or an effervescent tablet, and an alcoholic beverage. Accordingly, the beverage to which the dietary supplement is added, may be tea. The beverage may be coffee. The beverage may be juice (e.g. a fruit juice). The beverage may be lemonade. The beverage may be milk. The beverage may be cola. The beverage may be a drinkable milk product (e.g. drinkable yoghurt). The beverage may be a beverage obtainable by dissolving a sherbet powder or an effervescent tablet. The beverage may be an alcoholic beverage, e.g. beer or wine.

Other preferred edible compositions may be selected from the group consisting of a chewing gum, a candy, a bonbon, a fruit gum, a chocolate, and a composition for preparing a beverage (e.g. a coffee composition, a tea composition, a sherbet powder, or an effervescent tablet), and a beverage. Accordingly, the composition may be a chewing gum. The composition may be a candy. The composition may be a fruit gum. The composition may be a chocolate. The composition may be a composition for preparing a beverage. The composition may be a coffee composition (e.g. a composition comprising coffee, and further ingredients, e.g. milk powder, flavoring agents, and/or sweeteners, etc.). The composition may be a tea composition (e.g. a composition comprising tea leaves, or other components for a tea, e.g. fruit, and further ingredients, flavoring agents, and/or sweeteners, etc.). The composition may be a sherbet powder. The composition may be an effervescent tablet. The composition may be a beverage.

In a preferred embodiment, the composition is a chewing gum. Any chewing gum described throughout the present specification can be used.

The chewing gum comprises the compound selected from the group consisting of chitosan or a salt thereof, galactose, mannose, caffeine, and any combination thereof.

Accordingly, the compound may be caffeine. The compound may be mannose. The compound may be galactose. Preferably, the compound is chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein in accordance with the invention is any combination of the foregoing compounds. The salt may be in pharmaceutically acceptable quality.

In some embodiments, the chewing gum may comprise chitosan and an acid. Preferably, the acid is selected from the group consisting of, but not limited to, citric acid, acetic acid, ascorbic acid, malic acid, tartaric acid, any fruit acid, a food acidulant, and any combination thereof. More preferably, the acid is citric acid. When chewing, the acid lowers the pH so that at least part of the chitosan is converted into its cationic form. Accordingly, solubility of the chitosan in water is increased.

Also, the composition, which comprises the compound or the combination thereof, may be a beverage, as an example for a liquid composition. The beverage may be selected from the group consisting of tea, coffee, juice, lemonade, milk, cola, a drinkable milk product (e.g. a drinkable yoghurt), a beverage obtainable by dissolving a sherbet powder or an effervescent tablet, and an alcoholic beverage. Accordingly, the beverage which comprises the compound or the combination thereof, may be tea. The beverage may be coffee. The beverage may be juice (e.g. a fruit juice). The beverage may be lemonade. The beverage may be milk. The beverage may be cola. The beverage may be a drinkable milk product (e.g. drinkable yoghurt). The beverage may be a beverage obtainable by dissolving a sherbet powder or an effervescent tablet. The beverage may be an alcoholic beverage, e.g. beer or wine.

In some embodiments, the composition is selected from the group consisting of a solution, preferably a solution for spraying or gargling, a mouth rinse, a gel for application to the oral cavity, a nose drop formulation, a nose oil formulation, a nose spray, and a nose irrigation formulation. Accordingly, the composition may be a solution, preferably a solution for spraying or gargling. The composition may be a mouth rinse. The composition may be a gel for application to the oral cavity. The composition may be a nose drop formulation. The composition may be a nose oil formulation. The composition may be a nose spray. The composition may be a nose irrigation formulation.

In a preferred embodiment, the composition is or comprises a sustained delivery system. Preferably, the sustained delivery system is an oral or buccal sustained delivery system. Also described herein, according to embodiments of the invention, the drug delivery system may comprise a drug delivery implant, e.g. a refillable drug delivery implant. Several implantable devices may be contemplated for this purpose, e.g. a dental prothesis or a prosthesis fixation glue material.

Preferably, the composition is or comprises a solid or semi-solid dosage form. Accordingly, the composition is selected from the group consisting of a film, a layered structure of films, a tablet, a capsule, a gel body (e.g. a gel for application in the oral cavity), a chewing gum, a candy, a bonbon, a fruit gum, and a chocolate. Thus, the composition may be a film. The composition may be a layered structure of films. The composition may be a tablet. The composition may be a capsule. The composition may be a gel body (e.g. a gel for application in the oral cavity). The composition may be a chewing gum. The composition may be a candy. The composition may be a bonbon. The composition may be a fruit gum. The composition may be a chocolate.

Any one of the compositions described herein may comprise a particle. Preferably the particle is a microparticle or a nanoparticle; more preferably, the particle is a microparticle. Preferably, the particle, preferably a microparticle or a nanoparticle, more preferably a microparticle, comprises chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is a particle, preferably a microparticle or a nanoparticle, more preferably a microparticle, comprising any combination of the foregoing compounds. The salt may be in pharmaceutically acceptable quality.

In addition to the chitosan or salt thereof, the particle, preferably a microparticle or a nanoparticle, more preferably a microparticle, may comprise a further polymer or biopolymer. The polymer or biopolymer can include, but is not limited to, one or more of the following substances or classes of substances: proteins, polysaccharides, hydrocarbons, nucleic acids, aptamers, collagen, collagen-n-hydroxysuccinimide, fibrin, gelatin, albumin, alginates, blood plasma proteins, milk proteins, casein, protein-based polymers, hyaluronic acid, pectins, gummi arabicum and other gums, casein, whey proteins, gluten, starch, cellulose, synthetic polymers for pharmaceutical or cosmetic applications, like polylactic acid, polyglycolic acid, cell lysates of plants and microorganisms, copolymers and/or derivatives and/or mixtures and/or chemical modifications of said polymers and any combination thereof, with different material parameters such as chain length or molecular weight. The polymers may be utilized, for example, as excipients (e.g., for the incorporation and processing of active ingredients), as basic formulation substances (e.g., for cosmetics). Preferably, the further biopolymer is hyaluronic acid.

Accordingly, in a preferred embodiment, the particle, preferably a microparticle or a nanoparticle, more preferably a microparticle, further to chitosan or a salt thereof, comprises hyaluronic acid.

Any one of the compositions described herein may comprise a co-agglomerate of particles. Preferably, the co-agglomerate is a co-agglomerate of microparticles or a co-agglomerate of nanoparticles; more preferably the co-agglomerate is a co-agglomerate of microparticles. Preferably, the co-agglomerate of particles, preferably a co-agglomerate of microparticles or a co-agglomerate of nanoparticles, comprises chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. Also described herein is a co-agglomerate of particles, preferably a co-agglomerate of microparticles or a co-agglomerate of nanoparticles, more preferably a co-agglomerate of microparticles, comprising any combination of the foregoing compounds. The salt may be in pharmaceutically acceptable quality.

In addition to the chitosan or salt thereof, the co-agglomerate of particles, preferably a co-agglomerate of microparticles or a co-agglomerate of nanoparticles, more preferably a co-agglomerate of microparticles, may comprise a further polymer or biopolymer. The polymer or biopolymer can include, but is not limited to, one or more of the following substances or classes of substances: proteins, polysaccharides, hydrocarbons, nucleic acids, aptamers, collagen, collagen-n-hydroxysuccinimide, fibrin, gelatin, albumin, alginates, blood plasma proteins, milk proteins, casein, protein-based polymers, hyaluronic acid, pectins, gummi arabicum and other gums, casein, whey proteins, gluten, starch, cellulose, synthetic polymers for pharmaceutical or cosmetic applications, like polylactic acid, polyglycolic acid, cell lysates of plants and microorganisms, copolymers and/or derivatives and/or mixtures and/or chemical modifications of said polymers and any combination thereof, with different material parameters such as chain length or molecular weight. The polymers may be utilized, for example, as excipients (e.g., for the incorporation and processing of active ingredients), as basic formulation substances (e.g., for cosmetics). Preferably, the further biopolymer is hyaluronic acid. Accordingly, in a preferred embodiment, the co-agglomerate of particles, preferably a co-agglomerate of microparticles or a co-agglomerate of nanoparticles, more preferably a co-agglomerate of microparticles, further to chitosan or a salt thereof, comprises hyaluronic acid.

The particle, preferably a microparticle or a nanoparticle, more preferably a microparticle, or the co-agglomerate of particles, preferably a co-agglomerate of microparticles or a co-agglomerate of nanoparticles, more preferably a co-agglomerate of microparticles, may be comprised in a liquid or semi-solid composition. In particular, the particle, preferably a microparticle or a nanoparticle, more preferably a microparticle, or the co-agglomerate of particles, preferably a co-agglomerate of microparticles or a co-agglomerate of nanoparticles, more preferably a co-agglomerate of microparticles, is suspended in a liquid or semi-solid composition. Preferably, the particle, preferably a microparticle or nanoparticle, more preferably a microparticle, or co-agglomerate of particles, preferably a co-agglomerate of microparticles or a co-agglomerate of nanoparticles, more preferably a co-agglomerate of microparticles, is comprised, in particular suspended, in a composition for spraying or gargling, a mouth rinse, a gel for application to the oral cavity, a nose spray, a nose oil, a nose drop formulation, and a nasal irrigation formulation. The composition may be a composition for spraying or gargling. The composition may be a mouth rinse. The composition may be a gel for application to the oral cavity. The composition may be a nose spray. The composition may be a nose oil. The composition may be nose drop formulation. The composition may be a nasal irrigation formulation. The particle or co-agglomerate of particles, further to chitosan or a salt thereof, may comprise hyaluronic acid.

The particle, preferably a microparticle or nanoparticle, more preferably a microparticle, or the co-agglomerate, preferably a co-agglomerate of microparticles or nanoparticles, more preferably a co-agglomerate of microparticles, may be comprised in a composition suitable for treating a surface or skin. Preferably, the composition suitable for treating a surface or skin is selected from the group consisting of a disinfectant, a skin-caring composition (e.g. a lotion, a cream, a gel, a spray, a salve, an ointment or a powder), a soap, (e.g. a liquid soap), and a detergent formulation (e.g. a laundry detergent formulation or a dish detergent formulation). The composition may be a disinfectant. The composition may be a skin-caring composition. The skin-caring composition may comprise any cosmetic system or principle which permits the delivery of chitosan or a salt thereof or a derivative thereof. The composition may be a lotion. The composition may be a cream. The composition may be a gel. The composition may be a spray. The composition may be a salve. The composition may be an ointment. The composition may be a powder. The composition may be a soap. The composition may be a liquid soap. The composition may be a detergent formulation. The composition may be a laundry detergent formulation. The composition may be a dish detergent formulation. The particle or co-agglomerate of particles, further to chitosan or a salt thereof, may comprise hyaluronic acid, preferably, when the composition is a composition suitable for treating skin, e.g. a skin-caring composition, such as e.g. a lotion or cream. A use of the composition suitable for treating a surface or skin may comprise contacting the surface or skin with the composition suitable for treating a surface or skin.

The composition may be comprised in a solid mixture for manufacturing a nasal irrigation solution, a nose drop solution, a nose oil formulation, a solution for spraying or gargling, a mouth rinse, a gel for application to the oral cavity, or a beverage. The composition may be in form of a particle, preferably a microparticle or nanoparticle, more preferably a microparticle.

In any one of the compositions described herein, the chitosan or the salt thereof may have a degree of deacetylation of 60% or more. The chitosan or the salt thereof may have a degree of deacetylation of 70% or more. The chitosan or the salt thereof may have a degree of deacetylation of 80% or more. The chitosan or the salt thereof may have a degree of deacetylation of 90% or more. The chitosan or the salt thereof may have a degree of deacetylation of 95% or more.

In any one of the compositions described herein, the chitosan or the salt thereof may have a degree of deacetylation of from 30% to 60%. the chitosan or the salt thereof may have a degree of deacetylation of from 30% to 60%. The chitosan or the salt thereof may have a degree of deacetylation of from 35% to 55%. The chitosan or the salt thereof may have a degree of deacetylation of from 40% to 50%.

In any one of the compositions described herein, the chitosan or salt thereof may have a molecular weight of from 20000 Da to 460000 Da.

In some embodiments, the chitosan may be an oligochitosan having a molecular weight of below 20000 Da, or a salt of an oligochitosan having a molecular weight of below 20000 Da.

The method of increasing the binding of virus neutralizing antibodies may be carried out in vivo.

Also, the method of increasing the binding of virus neutralizing antibodies may be carried out in vitro. Accordingly, also disclosed herein is a diagnostic method, the method increasing the binding of virus neutralizing antibodies, and the method comprising contacting a fluid that contains a virus and virus neutralizing antibodies, wherein the virus neutralizing antibodies bind to said virus, with a compound selected from the group consisting of chitosan or a salt thereof, galactose, mannose, caffeine, and any combination thereof, as disclosed herein.

### Compound for Use in a Method of Increasing the Binding of Virus Neutralizing Antibodies

The present invention also relates to a compound selected from the group consisting of chitosan or a salt thereof, galactose, mannose, caffeine, and any combination thereof for use in a method of increasing the binding of virus neutralizing antibodies.

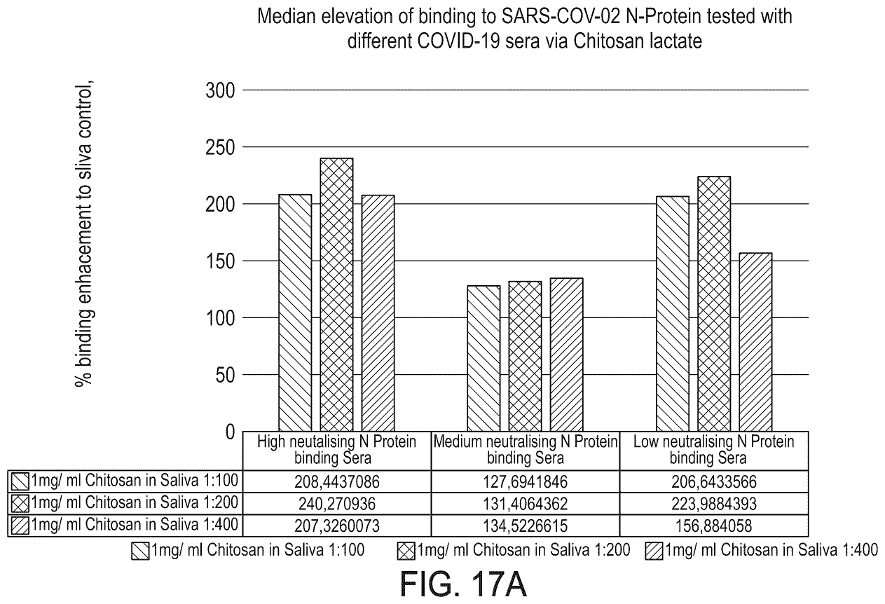

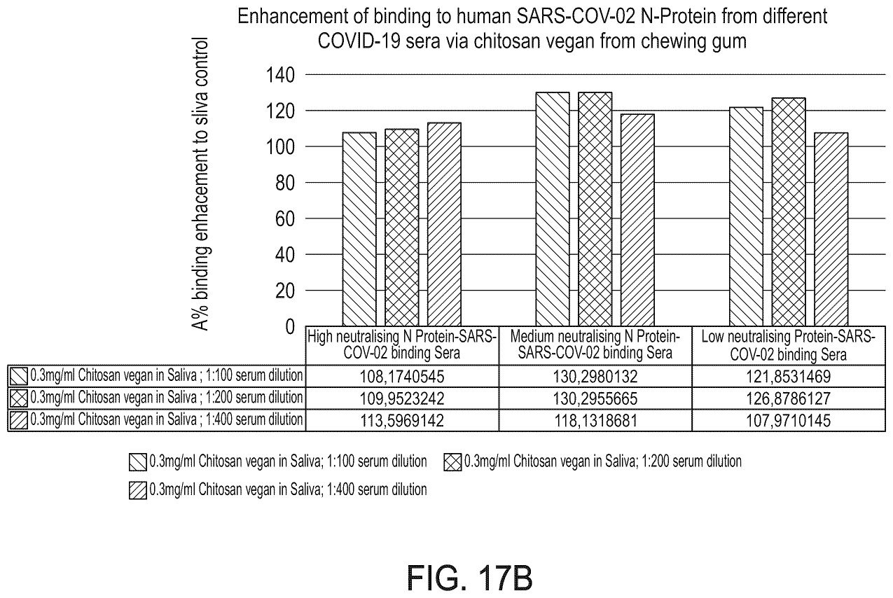

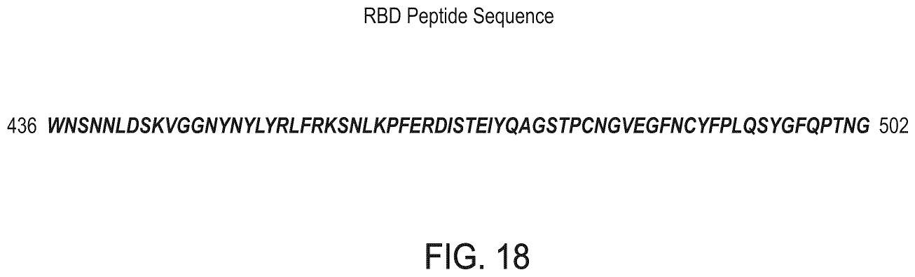

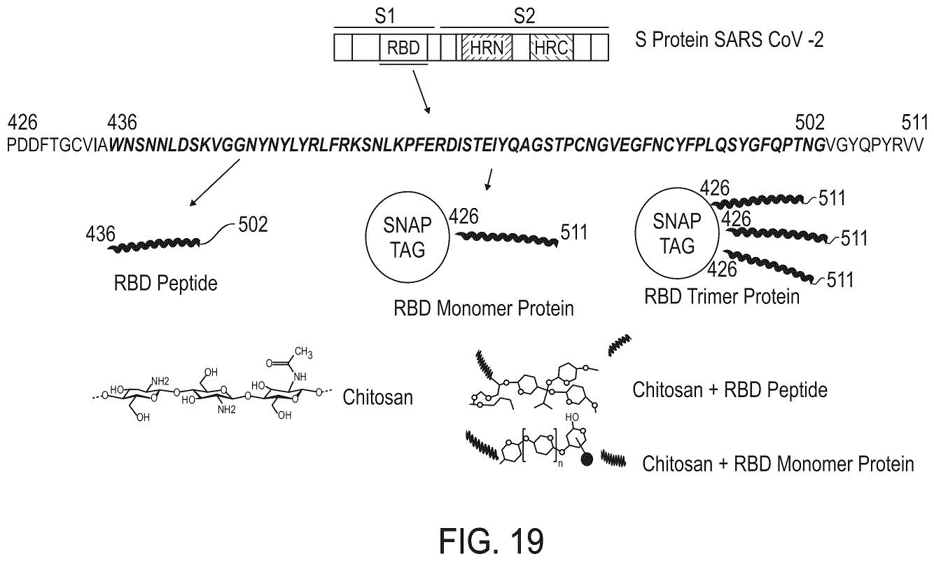

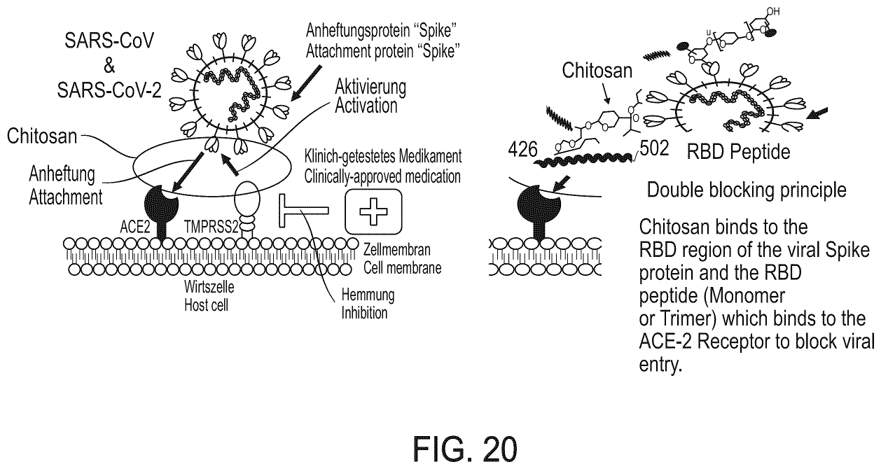

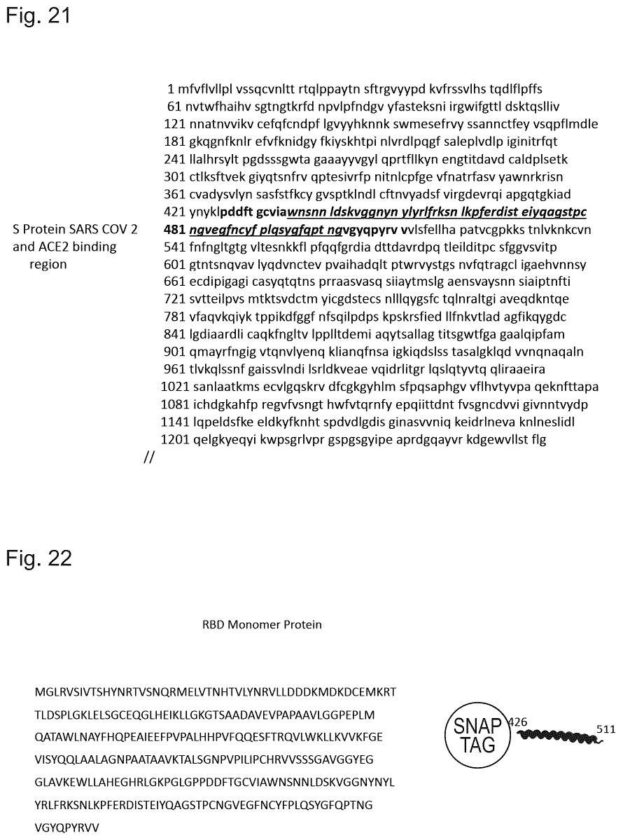

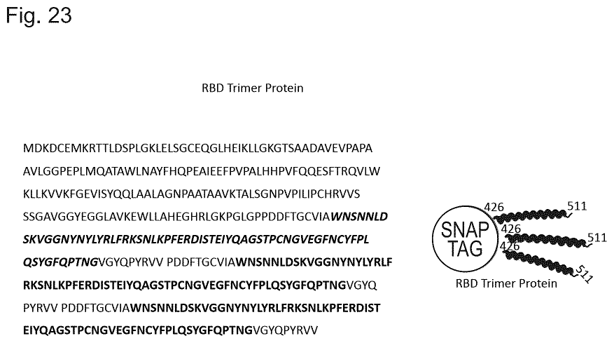

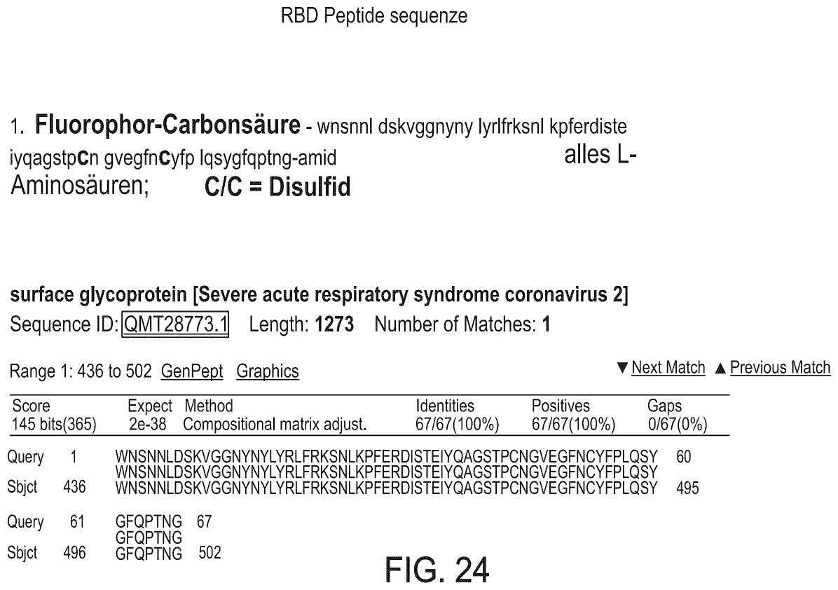

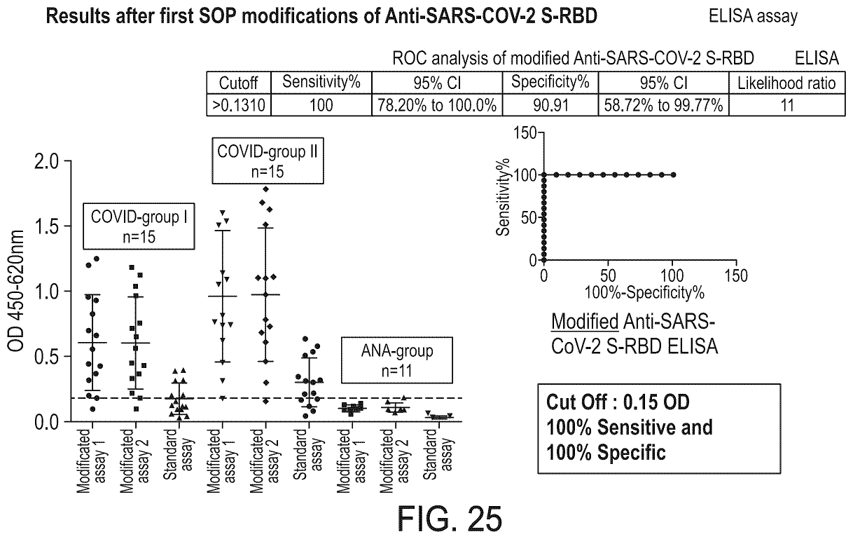

Surprisingly, the inventors could show that in particular low RBD SARS-CoV-2 binding antibodies exhibit an increased or enhanced binding with chitosan compared to samples without chitosan. This effect is probably exerted due to a stabilizing of the protein, which consequently may lead to an enhanced antibody binding, in particular of low RBD SARS-CoV-2 binding antibodies. This is also shown exemplary in FIGS. 15A, B. Additionally or alternatively, the inventors could show that antibodies specific for the nucleocapsid of SARS-CoV-2 or parts thereof exhibit an increased or enhanced binding with chitosan compared to samples without chitosan. This is also shown in FIGS. 17A, B.

In general, a person skilled in the art is able to define low, medium, and high binding antibodies by standard experimental procedures. According to the present invention, low-/medium-/high-binding antibodies have been defined by ELISA detection. On the basis of the detection limit of an ELISA assay, the group of low binding antibodies is defined as a group of 10-15% of the weakest binding antibodies, the group of medium binding antibodies is defined as a group of 15-80% of the moderate binding antibodies, the group of high binding antibodies is defined as a group of greater than 80% of the strongest binding antibodies.

In general, the use comprises that the virus neutralizing antibodies bind to said virus.

The use usually comprises contacting a fluid that contains a virus and virus neutralizing antibodies with the compound.

Preferably, the fluid is a bodily fluid. For example, the bodily fluid may be selected from the group consisting of blood, saliva and a mucous secretion. Accordingly, the bodily fluid may be saliva. The bodily fluid may be blood. The bodily fluid may be a mucous secretion. The mucous secretion may be a nasal secretion and/or a bronchial secretion. For example, the compound is preferably delivered to saliva or a mucous secretion of the respiratory tract, as respiratory viruses, such as e.g. SARS-CoV-2, after entering the human body are typically present in such bodily fluids.

In addition or alternatively, the method may comprise contacting a mucosa with the compound. In particular, when the virus is a respiratory virus, such as e.g. the virus SARS-CoV-2, the mucosa may be selected from the group consisting of mucosa of the nose, mucosa of the pharynx, mucosa of the mouth, and mucosa of the bronchia. Accordingly, the mucosa may be mucosa of the nose. The mucosa may be mucosa of the pharynx. The mucosa may be mucosa of the mouth. The mucosa may be mucosa of the bronchia.

The method may comprise contacting a skin of surface with the compound. In this regard, pathogens, e.g. viruses and bacteria, may be found on skin or a surface. Accordingly, a skin, e.g. the skin of humans, or any surface can be treated with any compound described herein, for any one of the uses and methods described herein.

The method may comprise contacting a wound or a burn with the compound. In this regard, pathogens, e.g. viruses and bacteria, may be found in a wound or a burn. Accordingly, a wound or a burn, e.g. a wound or a burn of human skin, can be treated with any compound described herein, for any one of the uses and methods described herein.

Exemplary viruses for the use in the method of increasing the binding of virus neutralizing antibodies may be selected from the group consisting of, but are not limited to, a coronavirus, preferably a human-pathogenic coronavirus, an influenza virus, a parainfluenza virus, a rhinovirus, a respiratory syncytial virus [RSV], an adenovirus, an orthomyxivirus, an Epstein-Barr virus, a herpes virus (e.g. herpes simplex), and a measles virus. Other exemplary viruses may be selected from the group consisting of influenza A, HIV-1; hepatitis A, B, C; HPV; EBV; norovirus; herpes simplex; cytomegalovirus; WSSV; RSV; plant virus like; TMV; AMV; TNV; BYMV; PSV; FMV; PVX; acute bee paralyisi virus; IAPV; chronic bee paralysis virus; varroa vectored virus; and in general any RNA and DNA virus. Infection with any virus or bacterium to which the compound (chitosan or salt thereof, galactose, mannose and/or caffeine) can bind can be prevented and/or treated. Illustrative viruses, bacteria, protozoan, and fungi include viruses, bacteria, protozoan, and fungi inducing diseases in humans, animals, bees and/or plants.

Further, any possible subunit of a virus or part of a virus or virion which is able exert an infection are also examples for the use in the method of increasing the binding of virus neutralizing antibodies as disclosed herein.

Further, any possible mutation of a virus which may develop, in particular during virus and host contact and interaction, or evolutionary alteration of a virus are also for the use in the method of increasing the binding of virus neutralizing antibodies as disclosed herein.

Preferably, the virus is a respiratory virus.

Preferably, the virus is selected from the group consisting of a coronavirus, preferably a human-pathogenic coronavirus, an influenza virus, a rhinovirus, and a respiratory syncytial virus [RSV].

Preferably, the virus is a coronavirus. As illustrative examples, the family of coronaviridae comprises an alphacoronavirus, a betacoronavirus, a deltacoronavirus, a gammacoronavirus, or an alphaletovirus. Preferably, the coronavirus is a human-pathogenic coronavirus. More preferably, the human-pathogenic coronavirus is a betacoronavirus. Even more preferably, the human-pathogenic coronavirus is selected from the group consisting of SARS-CoV-2, MERS-CoV and SARS-Cov-1.

Accordingly, the human-pathogenic coronavirus may be SARS-CoV-1. The human-pathogenic coronavirus may be MERS-CoV. Most preferably, the human-pathogenic coronavirus is SARS-CoV-2.

Preferably, the compound increases binding of SARS-CoV-2 neutralizing antibodies to the spike protein of SARS-CoV-2 or parts thereof or to the nucleocapsid protein of SARS-CoV-2 or parts thereof.

Accordingly, the RBD domain or parts thereof or to the nucleocapsid protein of SARS-CoV-2 or parts thereof is selected from the group consisting of the sequences as shown in SEQ ID NO:1, 2, 3, 4, 5, 6, and 7.

The compound for use in the method of increasing the binding of virus neutralizing antibodies may be a for use in a method of treating a virus infection or treating a disease caused by a virus infection.

The disease caused by a virus infection may be any disease caused by a virus, for example, a disease caused by a coronavirus, preferably a human-pathogenic coronavirus, an influenza virus, a parainfluenza virus, a rhinovirus, a respiratory syncytial virus [RSV], an adenovirus, an orthomyxivirus, an Epstein-Barr virus, a herpes virus (e.g. herpes simplex), and a measles virus.

Preferably, the disease is selected from the group consisting of COVID-19, MERS and SARS.

Accordingly, the disease may be SARS. The disease may be MERS. Most preferably, the disease is COVID-19.

According to the invention, the compound is selected from the group consisting of chitosan or a salt thereof, galactose, mannose, caffeine, and any combination thereof. The compound may be caffeine. The compound may be mannose. The compound may be galactose. Preferably, the compound is chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. The salt may be in pharmaceutically acceptable quality. Also described herein in accordance with the invention is any combination of the foregoing compounds.

The use may comprise using a composition that contains the compound selected from the group consisting of chitosan or a salt thereof, galactose, mannose, caffeine, and any combination thereof.

Accordingly, in the composition, the compound is selected from the group consisting of chitosan or a salt thereof, galactose, mannose, caffeine, and any combination thereof. In the composition, the compound may be caffeine. In the composition, the compound may be mannose. In the composition, the compound may be galactose. Preferably, in the composition the compound is chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. The salt may be in pharmaceutically acceptable quality. Also described herein in accordance with the invention is any combination of the foregoing compounds in the composition. The salt may be in pharmaceutically acceptable quality.

When the compound is chitosan, optionally the composition may further comprise an acid. Preferably, the acid is selected from the group consisting of, but not limited to, citric acid, acetic acid, ascorbic acid, malic acid, tartaric acid, any fruit acid, a food acidulant, and any combination thereof. More preferably, the acid is citric acid. The acid may lower the pH so that at least part of the chitosan is converted into its cationic form. Accordingly, solubility of the chitosan in water can be increased.

The composition may be any composition described throughout the present specification.

Method of Treating a Patient being Infected with a Virus

The present invention also relates to a method of treating a patient being infected with a virus, the method comprising contacting a fluid that contains a virus and virus neutralizing antibodies, wherein the virus neutralizing antibodies bind to said virus, with a compound selected from the group consisting of chitosan or a salt thereof, galactose, mannose, caffeine, and any combination thereof, wherein said compound increases the binding of virus neutralizing antibodies.

Preferably, the fluid is a bodily fluid. For example, the bodily fluid may be selected from the group consisting of blood, saliva and a mucous secretion. Accordingly, the bodily fluid may be saliva. The bodily fluid may be blood. The bodily fluid may be a mucous secretion. The mucous secretion may be a nasal secretion and/or a bronchial secretion. For example, the compound is preferably delivered to saliva or a mucous secretion of the respiratory tract, as respiratory viruses, such as e.g. SARS-CoV-2, after entering the human body are typically present in such bodily fluids.

In addition or alternatively, the method may comprise contacting a mucosa with the compound. In particular, when the virus is a respiratory virus, such as e.g. the virus SARS-CoV-2, the mucosa may be selected from the group consisting of mucosa of the nose, mucosa of the pharynx, mucosa of the mouth, and mucosa of the bronchia. Accordingly, the mucosa may be mucosa of the nose. The mucosa may be mucosa of the pharynx. The mucosa may be mucosa of the mouth. The mucosa may be mucosa of the bronchia.

The method may comprise contacting a skin of surface with the compound. In this regard, pathogens, e.g. viruses and bacteria, may be found on skin or a surface. Accordingly, a skin, e.g. the skin of humans, or any surface can be treated with any compound described herein, for any one of the uses and methods described herein.

The method may comprise contacting a wound or a burn with the compound. In this regard, pathogens, e.g. viruses and bacteria, may be found in a wound or a burn. Accordingly, a wound or a burn, e.g. a wound or a burn of human skin, can be treated with any compound described herein, for any one of the uses and methods described herein.

Exemplary viruses for the use in the method of treating a patient being infected with a virus may be selected from the group consisting of, but are not limited to, a coronavirus, preferably a human-pathogenic coronavirus, an influenza virus, a parainfluenza virus, a rhinovirus, a respiratory syncytial virus [RSV], an adenovirus, an orthomyxivirus, an Epstein-Barr virus, a herpes virus (e.g. herpes simplex), and a measles virus. Other exemplary viruses may be selected from the group consisting of influenza A, HIV-1; hepatitis A, B, C; HPV; EBV; norovirus; herpes simplex; cytomegalovirus; WSSV; RSV; plant virus like; TMV; AMV; TNV; BYMV; PSV; FMV; PVX; acute bee paralyisi virus; IAPV; chronic bee paralysis virus; varroa vectored virus; and in general any RNA and DNA virus. Infection with any virus or bacterium to which the compound (chitosan or salt thereof, galactose, mannose and/or caffeine) can bind can be prevented and/or treated. Illustrative viruses, bacteria, protozoan, and fungi include viruses and bacteria inducing diseases in humans, animals, bees and/or plants.

Further, any possible subunit of a virus or part of a virus or virion which is able exert an infection are also examples for the use in the method of treating a patient being infected with a virus as disclosed herein.

Further, any possible mutation of a virus which may develop, in particular during virus and host contact and interaction, or evolutionary alteration of a virus are also examples for the use in the method of treating a patient being infected with a virus as disclosed herein

Preferably, the virus is a respiratory virus.

Preferably, the virus is selected from the group consisting of a coronavirus, preferably a human-pathogenic coronavirus, an influenza virus, a rhinovirus, and a respiratory syncytial virus [RSV].

Preferably, the virus is a coronavirus. As illustrative examples, the family of coronaviridae comprises an alphacoronavirus, a betacoronavirus, a deltacoronavirus, a gammacoronavirus, or an alphaletovirus. Preferably, the coronavirus is a human-pathogenic coronavirus. More preferably, the human-pathogenic coronavirus is a betacoronavirus. Even more preferably, the human-pathogenic coronavirus is selected from the group consisting of SARS-CoV-2, MERS-CoV and SARS-Cov-1.

Accordingly, the human-pathogenic coronavirus may be SARS-CoV-1. The human-pathogenic coronavirus may be MERS-CoV. Most preferably, the human-pathogenic coronavirus is SARS-CoV-2.

The method of treating a patient being infected with a virus may be a method of treating a virus infection or treating a disease caused by a virus infection.

The disease caused by a virus infection may be any disease caused by a virus, for example, a disease caused by a coronavirus, preferably a human-pathogenic coronavirus, an influenza virus, a parainfluenza virus, a rhinovirus, a respiratory syncytial virus [RSV], an adenovirus, an orthomyxivirus, an Epstein-Barr virus, a herpes virus (e.g. herpes simplex), and a measles virus.

Preferably, the disease is selected from the group consisting of COVID-19, MERS and SARS.

Accordingly, the disease may be SARS. The disease may be MERS. Most preferably, the disease is COVID-19.

According to the invention, the compound is selected from the group consisting of chitosan or a salt thereof, galactose, mannose, caffeine, and any combination thereof. The compound may be caffeine. The compound may be mannose. The compound may be galactose. Preferably, the compound is chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. The salt may be in pharmaceutically acceptable quality. Also described herein in accordance with the invention is any combination of the foregoing compounds.

The method may comprise contacting the fluid that contains the virus and virus neutralizing antibodies with a composition that contains the compound selected from the group consisting of chitosan or a salt thereof, galactose, mannose, caffeine, and any combination thereof.

Accordingly, in the composition, the compound is selected from the group consisting of chitosan or a salt thereof, galactose, mannose, caffeine, and any combination thereof. In the composition, the compound may be caffeine. In the composition, the compound may be mannose. In the composition, the compound may be galactose. Preferably, in the composition the compound is chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. The salt may be in pharmaceutically acceptable quality. Also described herein in accordance with the invention is any combination of the foregoing compounds in the composition. The salt may be in pharmaceutically acceptable quality.

When the compound is chitosan, optionally the composition may further comprise an acid. Preferably, the acid is selected from the group consisting of, but not limited to, citric acid, acetic acid, ascorbic acid, malic acid, tartaric acid, any fruit acid, a food acidulant, and any combination thereof. More preferably, the acid is citric acid. The acid may lower the pH so that at least part of the chitosan is converted into its cationic form. Accordingly, solubility of the chitosan in water can be increased.

The composition may be any composition described throughout the present specification.

Compound for Use in a Method of Treating a Patient being Infected with a Virus

The present invention also relates to compound for use in a method of treating a subject being infected with a virus, the method comprising contacting a fluid that contains a virus and virus neutralizing antibodies, wherein the virus neutralizing antibodies bind to said virus, with the compound, wherein the compound is selected from the group consisting of chitosan or a salt thereof, galactose, mannose, caffeine, and any combination thereof, wherein said compound increases the binding of virus neutralizing antibodies.

In general, the use comprises that the virus neutralizing antibodies bind to said virus.

The use usually comprises contacting a fluid that contains a virus and virus neutralizing antibodies with the compound.

Preferably, the fluid is a bodily fluid. For example, the bodily fluid may be selected from the group consisting of blood, saliva and a mucous secretion. Accordingly, the bodily fluid may be saliva. The bodily fluid may be blood. The bodily fluid may be a mucous secretion. The mucous secretion may be a nasal secretion and/or a bronchial secretion. For example, the compound is preferably delivered to saliva or a mucous secretion of the respiratory tract, as respiratory viruses, such as e.g. SARS-CoV-2, after entering the human body are typically present in such bodily fluids.

In addition or alternatively, the method may comprise contacting a mucosa with the compound. In particular, when the virus is a respiratory virus, such as e.g. the virus SARS-CoV-2, the mucosa may be selected from the group consisting of mucosa of the nose, mucosa of the pharynx, mucosa of the mouth, and mucosa of the bronchia. Accordingly, the mucosa may be mucosa of the nose. The mucosa may be mucosa of the pharynx. The mucosa may be mucosa of the mouth. The mucosa may be mucosa of the bronchia.

The method may comprise contacting a skin of surface with the compound. In this regard, pathogens, e.g. viruses and bacteria, may be found on skin or a surface. Accordingly, a skin, e.g. the skin of humans, or any surface can be treated with any compound described herein, for any one of the uses and methods described herein.

The method may comprise contacting a wound or a burn with the compound. In this regard, pathogens, e.g. viruses and bacteria, may be found in a wound or a burn. Accordingly, a wound or a burn, e.g. a wound or a burn of human skin, can be treated with any compound described herein, for any one of the uses and methods described herein.

Exemplary viruses for the use in the method of increasing the binding of virus neutralizing antibodies may be selected from the group consisting of, but are not limited to, a coronavirus, preferably a human-pathogenic coronavirus, an influenza virus, a parainfluenza virus, a rhinovirus, a respiratory syncytial virus [RSV], an adenovirus, an orthomyxivirus, an Epstein-Barr virus, a herpes virus (e.g. herpes simplex), and a measles virus. Other exemplary viruses may be selected from the group consisting of influenza A, HIV-1; hepatitis A, B, C; HPV; EBV; norovirus; herpes simplex; cytomegalovirus; WSSV; RSV; plant virus like; TMV; AMV; TNV; BYMV; PSV; FMV; PVX; acute bee paralyisi virus; IAPV; chronic bee paralysis virus; varroa vectored virus; and in general any RNA and DNA virus. Infection with any virus or bacterium to which the compound (chitosan or salt thereof, galactose, mannose and/or caffeine) can bind can be prevented and/or treated. Illustrative viruses, bacteria, protozoan, and fungi include viruses, bacteria, protozoan, and fungi inducing diseases in humans, animals, bees and/or plants.

Further, any possible subunit of a virus or part of a virus or virion which is able exert an infection are also examples for the use in the method of increasing the binding of virus neutralizing antibodies as disclosed herein.

Further, any possible mutation of a virus which may develop, in particular during virus and host contact and interaction, or evolutionary alteration of a virus are also for the use in the method of increasing the binding of virus neutralizing antibodies as disclosed herein.

Preferably, the virus is a respiratory virus.

Preferably, the virus is selected from the group consisting of a coronavirus, preferably a human-pathogenic coronavirus, an influenza virus, a rhinovirus, and a respiratory syncytial virus [RSV].

Preferably, the virus is a coronavirus. As illustrative examples, the family of coronaviridae comprises an alphacoronavirus, a betacoronavirus, a deltacoronavirus, a gammacoronavirus, or an alphaletovirus. Preferably, the coronavirus is a human-pathogenic coronavirus. More preferably, the human-pathogenic coronavirus is a betacoronavirus. Even more preferably, the human-pathogenic coronavirus is selected from the group consisting of SARS-CoV-2, MERS-CoV and SARS-Cov-1.

Accordingly, the human-pathogenic coronavirus may be SARS-CoV-1. The human-pathogenic coronavirus may be MERS-CoV. Most preferably, the human-pathogenic coronavirus is SARS-CoV-2.

Preferably, the compound increases binding of SARS-CoV-2 neutralizing antibodies to the spike protein of SARS-CoV-2 or parts thereof or to the nucleocapsid protein of SARS-CoV-2 or parts thereof.

Accordingly, the RBD domain or parts thereof or to the nucleocapsid protein of SARS-CoV-2 or parts thereof is selected from the group consisting of the sequences as shown in SEQ ID NO:1, 2, 3, 4, 5, 6, and 7.

The compound for use in the method of increasing the binding of virus neutralizing antibodies may be a for use in a method of treating a virus infection or treating a disease caused by a virus infection.

The disease caused by a virus infection may be any disease caused by a virus, for example, a disease caused by a coronavirus, preferably a human-pathogenic coronavirus, an influenza virus, a parainfluenza virus, a rhinovirus, a respiratory syncytial virus [RSV], an adenovirus, an orthomyxivirus, an Epstein-Barr virus, a herpes virus (e.g. herpes simplex), and a measles virus.

Preferably, the disease is selected from the group consisting of COVID-19, MERS and SARS.

Accordingly, the disease may be SARS. The disease may be MERS. Most preferably, the disease is COVID-19.

According to the invention, the compound is selected from the group consisting of chitosan or a salt thereof, galactose, mannose, caffeine, and any combination thereof. The compound may be caffeine. The compound may be mannose. The compound may be galactose. Preferably, the compound is chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. The salt may be in pharmaceutically acceptable quality. Also described herein in accordance with the invention is any combination of the foregoing compounds.

The use may comprise using a composition that contains the compound selected from the group consisting of chitosan or a salt thereof, galactose, mannose, caffeine, and any combination thereof.

Accordingly, in the composition, the compound is selected from the group consisting of chitosan or a salt thereof, galactose, mannose, caffeine, and any combination thereof. In the composition, the compound may be caffeine. In the composition, the compound may be mannose. In the composition, the compound may be galactose. Preferably, in the composition the compound is chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. The salt may be in pharmaceutically acceptable quality. Also described herein in accordance with the invention is any combination of the foregoing compounds in the composition. The salt may be in pharmaceutically acceptable quality.

When the compound is chitosan, optionally the composition may further comprise an acid. Preferably, the acid is selected from the group consisting of, but not limited to, citric acid, acetic acid, ascorbic acid, malic acid, tartaric acid, any fruit acid, a food acidulant, and any combination thereof. More preferably, the acid is citric acid. The acid may lower the pH so that at least part of the chitosan is converted into its cationic form. Accordingly, solubility of the chitosan in water can be increased.

The composition may be any composition described throughout the present specification.

### An In Vitro Method of Detecting Virus-Specific Antibodies

The present invention also relates to an in vitro method of detecting virus-specific antibodies in a sample obtained from a subject, the method comprising contacting a fluid that contains virus-specific antibodies with virus protein under conditions that allow binding of the virus-specific antibody to the virus protein, with a compound selected from the group consisting of chitosan or a salt thereof, galactose, mannose, caffeine, and any combination thereof, thereby detecting said virus-specific antibodies.

Preferably, the virus-specific antibodies are indicative for a virus infection.

Preferably, the fluid is a bodily fluid. For example, the bodily fluid may be selected from the group consisting of blood, saliva and a mucous secretion. Accordingly, the bodily fluid may be saliva. The bodily fluid may be blood. The bodily fluid may be a mucous secretion. The mucous secretion may be a nasal secretion and/or a bronchial secretion. For example, the compound is preferably delivered to saliva or a mucous secretion of the respiratory tract, as respiratory viruses, such as e.g. SARS-CoV-2, after entering the human body are typically present in such bodily fluids.

In addition or alternatively, the method may comprise contacting a mucosa with the compound. In particular, when the virus is a respiratory virus, such as e.g. the virus SARS-CoV-2, the mucosa may be selected from the group consisting of mucosa of the nose, mucosa of the pharynx, mucosa of the mouth, and mucosa of the bronchia. Accordingly, the mucosa may be mucosa of the nose. The mucosa may be mucosa of the pharynx. The mucosa may be mucosa of the mouth. The mucosa may be mucosa of the bronchia.

The method may comprise contacting a skin of surface with the compound. In this regard, pathogens, e.g. viruses and bacteria, may be found on skin or a surface. Accordingly, a skin, e.g. the skin of humans, or any surface can be treated with any compound described herein, for any one of the uses and methods described herein.

The method may comprise contacting a wound or a burn with the compound. In this regard, pathogens, e.g. viruses and bacteria, may be found in a wound or a burn. Accordingly, a wound or a burn, e.g. a wound or a burn of human skin, can be treated with any compound described herein, for any one of the uses and methods described herein.

Exemplary viruses for an in vitro method of detecting virus-specific antibodies in a sample obtained from a subject may be selected from the group consisting of, but are not limited to, a coronavirus, preferably a human-pathogenic coronavirus, an influenza virus, a parainfluenza virus, a rhinovirus, a respiratory syncytial virus [RSV], an adenovirus, an orthomyxivirus, an Epstein-Barr virus, a herpes virus (e.g. herpes simplex), and a measles virus. Other exemplary viruses may be selected from the group consisting of influenza A, HIV-1; hepatitis A, B, C; HPV; EBV; norovirus; herpes simplex; cytomegalovirus; WSSV; RSV; plant virus like; TMV; AMV; TNV; BYMV; PSV; FMV; PVX; acute bee paralyisi virus; IAPV; chronic bee paralysis virus; varroa vectored virus; and in general any RNA and DNA virus. Infection with any virus or bacterium to which the compound (chitosan or salt thereof, galactose, mannose and/or caffeine) can bind can be prevented and/or treated. Illustrative viruses, bacteria, protozoan, and fungi include viruses, bacteria, protozoan, and fungi inducing diseases in humans, animals, bees and/or plants.

Further, any possible subunit of a virus or part of a virus or virion which is able exert an infection are also examples for the use in the method of increasing the binding of virus neutralizing antibodies as disclosed herein.

Further, any possible mutation of a virus which may develop, in particular during virus and host contact and interaction, or evolutionary alteration of a virus are also for the use in the method of increasing the binding of virus neutralizing antibodies as disclosed herein.

Preferably, the virus is a respiratory virus.

Preferably, the virus is selected from the group consisting of a coronavirus, preferably a human-pathogenic coronavirus, an influenza virus, a rhinovirus, and a respiratory syncytial virus [RSV].

Preferably, the virus is a coronavirus. As illustrative examples, the family of coronaviridae comprises an alphacoronavirus, a betacoronavirus, a deltacoronavirus, a gammacoronavirus, or an alphaletovirus. Preferably, the coronavirus is a human-pathogenic coronavirus. More preferably, the human-pathogenic coronavirus is a betacoronavirus. Even more preferably, the human-pathogenic coronavirus is selected from the group consisting of SARS-CoV-2, MERS-CoV and SARS-Cov-1.

Accordingly, the human-pathogenic coronavirus may be SARS-CoV-1. The human-pathogenic coronavirus may be MERS-CoV. Most preferably, the human-pathogenic coronavirus is SARS-CoV-2.

Preferably, the virus-specific protein is a SARS-CoV-2 specific protein.

Accordingly, the SARS-CoV-2 specific protein is the spike protein, preferably the RBD domain of the spike protein or parts thereof or to the nucleocapsid protein of SARS-CoV-2 or parts thereof.

Preferably, the compound increases binding of SARS-CoV-2-specific antibodies to the spike protein of SARS-CoV-2, preferably the RBD domain of the spike protein of SARS-CoV-2 or parts thereof or to the nucleocapsid protein of SARS-CoV-2 or parts thereof.

Accordingly, the RBD domain or parts thereof or to the nucleocapsid protein of SARS-CoV-2 or parts thereof is selected from the group consisting of the sequences as shown in SEQ ID NO:1, 2, 3, 4, 5, 6, and 7.

The composition may be any composition described throughout the present specification.

### Compound for Use in a Method of Detecting Virus-Specific Antibodies in a Subject

The present invention also relates to a compound selected from the group consisting of chitosan or a salt thereof, galactose, mannose, caffeine, and any combination thereof for use in a method of detecting of virus-specific antibodies in a subject.

Preferably, the method of detecting comprises contacting a fluid that contains virus-specific antibodies with a virus protein with the compound.

Preferably, the fluid is a bodily fluid. For example, the bodily fluid may be selected from the group consisting of blood, saliva and a mucous secretion. Accordingly, the bodily fluid may be saliva. The bodily fluid may be blood. The bodily fluid may be a mucous secretion. The mucous secretion may be a nasal secretion and/or a bronchial secretion. For example, the compound is preferably delivered to saliva or a mucous secretion of the respiratory tract, as respiratory viruses, such as e.g. SARS-CoV-2, after entering the human body are typically present in such bodily fluids.

In addition or alternatively, the compound for use in a method of detecting virus-specific antibodies may comprise contacting a mucosa with the compound. In particular, when the virus is a respiratory virus, such as e.g. the virus SARS-CoV-2, the mucosa may be selected from the group consisting of mucosa of the nose, mucosa of the pharynx, mucosa of the mouth, and mucosa of the bronchia. Accordingly, the mucosa may be mucosa of the nose. The mucosa may be mucosa of the pharynx. The mucosa may be mucosa of the mouth. The mucosa may be mucosa of the bronchia.

The compound for use in a method of detecting virus-specific antibodies may comprise contacting a skin of surface with the compound. In this regard, pathogens, e.g. viruses and bacteria, may be found on skin or a surface. Accordingly, a skin, e.g. the skin of humans, or any surface can be treated with any compound described herein, for any one of the uses and methods described herein.

The compound for use in a method of detecting virus-specific antibodies may comprise contacting a wound or a burn with the compound. In this regard, pathogens, e.g. viruses and bacteria, may be found in a wound or a burn. Accordingly, a wound or a burn, e.g. a wound or a burn of human skin, can be treated with any compound described herein, for any one of the uses and methods described herein.

Exemplary viruses for the compound for use in a method of detecting virus-specific antibodies may be selected from the group consisting of, but are not limited to, a coronavirus, preferably a human-pathogenic coronavirus, an influenza virus, a parainfluenza virus, a rhinovirus, a respiratory syncytial virus [RSV], an adenovirus, an orthomyxivirus, an Epstein-Barr virus, a herpes virus (e.g. herpes simplex), and a measles virus. Other exemplary viruses may be selected from the group consisting of influenza A, HIV-1; hepatitis A, B, C; HPV; EBV; norovirus; herpes simplex; cytomegalovirus; WSSV; RSV; plant virus like; TMV; AMV; TNV; BYMV; PSV; FMV; PVX; acute bee paralyisi virus; IAPV; chronic bee paralysis virus; varroa vectored virus; and in general any RNA and DNA virus. Infection with any virus or bacterium to which the compound (chitosan or salt thereof, galactose, mannose and/or caffeine) can bind can be prevented and/or treated. Illustrative viruses, bacteria, protozoan, and fungi include viruses, bacteria, protozoan, and fungi inducing diseases in humans, animals, bees and/or plants.

Further, any possible subunit of a virus or part of a virus or virion which is able exert an infection are also examples for the use in the method of increasing the binding of virus neutralizing antibodies as disclosed herein.

Further, any possible mutation of a virus which may develop, in particular during virus and host contact and interaction, or evolutionary alteration of a virus are also for the use in the method of increasing the binding of virus neutralizing antibodies as disclosed herein.

Preferably, the virus is a respiratory virus.

Preferably, the virus is selected from the group consisting of a coronavirus, preferably a human-pathogenic coronavirus, an influenza virus, a rhinovirus, and a respiratory syncytial virus [RSV].

Preferably, the virus is a coronavirus. As illustrative examples, the family of coronaviridae comprises an alphacoronavirus, a betacoronavirus, a deltacoronavirus, a gammacoronavirus, or an alphaletovirus. Preferably, the coronavirus is a human-pathogenic coronavirus. More preferably, the human-pathogenic coronavirus is a betacoronavirus. Even more preferably, the human-pathogenic coronavirus is selected from the group consisting of SARS-CoV-2, MERS-CoV and SARS-Cov-1.

Accordingly, the human-pathogenic coronavirus may be SARS-CoV-1. The human-pathogenic coronavirus may be MERS-CoV. Most preferably, the human-pathogenic coronavirus is SARS-CoV-2.

Preferably, the virus-specific protein is a SARS-CoV-2 specific protein.

Accordingly, the SARS-CoV-2 specific protein is the spike protein, preferably the RBD domain of the spike protein or parts thereof or to the nucleocapsid protein of SARS-CoV-2 or parts thereof.

Preferably, the compound increases binding of SARS-CoV-2-specific antibodies to the spike protein of SARS-CoV-2, preferably the RBD domain of the spike protein of SARS-CoV-2 or parts thereof or to the nucleocapsid protein of SARS-CoV-2 or parts thereof.

Accordingly, the RBD domain or parts thereof or to the nucleocapsid protein of SARS-CoV-2 or parts thereof is selected from the group consisting of the sequences as shown in SEQ ID NO:1, 2, 3, 4, 5, 6, and 7.

The composition may be any composition described throughout the present specification.

### A Kit for the Detection of Virus-Specific Antibodies

The present invention also relates to a kit for the detection of virus-specific antibodies, the kit comprising a virus protein and a compound selected from the group consisting of chitosan or a salt thereof, galactose, mannose, caffeine, and any combination thereof.

Exemplary viruses for the kit of detection of virus-specific antibodies may be selected from the group consisting of, but are not limited to, a coronavirus, preferably a human-pathogenic coronavirus, an influenza virus, a parainfluenza virus, a rhinovirus, a respiratory syncytial virus [RSV], an adenovirus, an orthomyxivirus, an Epstein-Barr virus, a herpes virus (e.g. herpes simplex), and a measles virus. Other exemplary viruses may be selected from the group consisting of influenza A, HIV-1; hepatitis A, B, C; HPV; EBV; norovirus; herpes simplex; cytomegalovirus; WSSV; RSV; plant virus like; TMV; AMV; TNV; BYMV; PSV; FMV; PVX; acute bee paralyisi virus; IAPV; chronic bee paralysis virus; varroa vectored virus; and in general any RNA and DNA virus. Infection with any virus or bacterium to which the compound (chitosan or salt thereof, galactose, mannose and/or caffeine) can bind can be prevented and/or treated. Illustrative viruses, bacteria, protozoan, and fungi include viruses, bacteria, protozoan, and fungi inducing diseases in humans, animals, bees and/or plants.

Further, any possible subunit of a virus or part of a virus or virion which is able exert an infection are also examples for the use in the method of increasing the binding of virus neutralizing antibodies as disclosed herein.

Further, any possible mutation of a virus which may develop, in particular during virus and host contact and interaction, or evolutionary alteration of a virus are also for the use in the method of increasing the binding of virus neutralizing antibodies as disclosed herein.

Preferably, the virus is a respiratory virus.

Preferably, the virus is selected from the group consisting of a coronavirus, preferably a human-pathogenic coronavirus, an influenza virus, a rhinovirus, and a respiratory syncytial virus [RSV].

Preferably, the virus is a coronavirus. As illustrative examples, the family of coronaviridae comprises an alphacoronavirus, a betacoronavirus, a deltacoronavirus, a gammacoronavirus, or an alphaletovirus. Preferably, the coronavirus is a human-pathogenic coronavirus. More preferably, the human-pathogenic coronavirus is a betacoronavirus. Even more preferably, the human-pathogenic coronavirus is selected from the group consisting of SARS-CoV-2, MERS-CoV and SARS-Cov-1.

Accordingly, the human-pathogenic coronavirus may be SARS-CoV-1. The human-pathogenic coronavirus may be MERS-CoV. Most preferably, the human-pathogenic coronavirus is SARS-CoV-2.

Preferably, the compound is chitosan or a salt thereof.

According to the invention, the compound is selected from the group consisting of chitosan or a salt thereof, galactose, mannose, caffeine, and any combination thereof. The compound may be caffeine. The compound may be mannose. The compound may be galactose. Preferably, the compound is chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. The salt may be in pharmaceutically acceptable quality. Also described herein in accordance with the invention is any combination of the foregoing compounds.

Accordingly, in the composition, the compound is selected from the group consisting of chitosan or a salt thereof, galactose, mannose, caffeine, and any combination thereof. In the composition, the compound may be caffeine. In the composition, the compound may be mannose. In the composition, the compound may be galactose. Preferably, in the composition the compound is chitosan or a salt thereof. A salt of chitosan may be selected from the group consisting of chitosan lactate, chitosan acetate, and chitosan hydrochloride, and any combination thereof. Accordingly, the salt may be chitosan lactate. The salt may be chitosan acetate. The salt may be chitosan hydrochloride. The salt may be in pharmaceutically acceptable quality. Also described herein in accordance with the invention is any combination of the foregoing compounds in the composition. The salt may be in pharmaceutically acceptable quality.

When the compound is chitosan, optionally the composition may further comprise an acid. Preferably, the acid is selected from the group consisting of, but not limited to, citric acid, acetic acid, ascorbic acid, malic acid, tartaric acid, any fruit acid, a food acidulant, and any combination thereof. More preferably, the acid is citric acid. The acid may lower the pH so that at least part of the chitosan is converted into its cationic form. Accordingly, solubility of the chitosan in water can be increased.

The composition may be any composition described throughout the present specification.

Preferably, the kit is suitable for conducting an ELISA assay.

Further preferred, the kit is suitable for the detection of SARS-CoV-2 antibodies.

Preferably, the kit is suitable for the detection of antibodies against SARS-CoV-2 spike protein, preferably the spike RBD domain or parts thereof or to the nucleocapsid protein of SARS-CoV-2 or parts thereof.

Further preferred, the compound of the kit increases binding of SARS-CoV-2-specific antibodies to the spike protein of SARS-CoV-2, preferably the RBD domain of the spike protein of SARS-CoV-2 or parts thereof or to the nucleocapsid protein of SARS-CoV-2 or parts thereof.

Preferably, the RBD domain or parts thereof or to the nucleocapsid protein of SARS-CoV-2 or parts thereof is selected from the group consisting of SEQ ID NO:1, 2, 3, 4, 5, 6, and 7.

Representative Non-limiting Embodiments and Examples 1. In one preferred embodiment, in one-time delivery modus Chitosan solution is applied to mucosa of Pharynx by means of spraying or gargling to achieve a therapeutic dose at Pharynx mucosa. The surface area of the oral cavity is around 200 cm². The added surface area of the different parts of pharynx anatomy covered by mucosa is smaller than this. We will assume a value of 100 cm² as an upper limit, however, understanding that this value may be drastically change from individuum to individuum. Nevertheless it will work for orientation purposes. From protein chemistry is known that about 1 mg (or, at least, a value of that order of magnitude) protein is able to cover 1 m². Chitosan is a carbon hydrate and possesses an extended water binding capacity. Its surface density will be lower than that of proteins. The geometric thickness of mucosa is between about 0.1 and 0.5 millimeter. This results in about 5 cm 3 volume of pharynx mucosa. This is an estimated upper limit value, but good enough for relating the quantitative considerations to.

A typical value of the molecular weight of Chitosan is 1 million Dalton. This means 1 microgram is containing about 6*10¹¹ molecules. (for a lower molecular weight or an oligochitosan the absolute molecule numbers are larger, respectively) Infection with corona virus SARS-CoV-2 is successfully achieved after taking up of about 500 viruses more or less simultaneously by the pharynx mucosa (as compared to 100 or less in case of influenca or 5 in case of Norovirus). One SARS-CoV-2 virus may contain a few hundred to thousand spike proteins containing the RBD region in charge for getting into contact with target cells and being part of the genome transfer induction system. Nevertheless, in any case, one thousand Chitosan molecules per virus could block the surface spikes completely, a smaller number would already affect their function drastically. The same type of consideration is valid for the ACE2 target receptor density on submucosa cell lines. Nevertheless, the ACE2 enzyme expression density on submucosa membranes is highly flexible and depends on a whole series of organismic controls and inter-individual differences. For the sake of present discussion the very rough estimation of order of magnitude of the virus surface receptor density is sufficient. If this side is blocked by Chitosan the chain of infection is broken.

To obtain a very rough estimation let us assume for the moment one gets 1 microgram (6*10¹¹ molecules of Chitosan of molecular weight of 1 Mio Dalton) into a pharynx mucosa of 5 ml (10 cm²*0.5 mm thickness). If we further assume that each SARS-CoV-2 virus would express about 1000 RBD regions on its surface the amount of Chitosan would be sufficient for blocking of 6*10⁸ viruses. Assuming that about 500 SARS-CoV-2 viruses are enough to trigger the infection one would have a large safety distance which could cope with a much larger virus load as well as with greater scatterings of Chitosan load of mucosa, of inactivation of loaded Chitosan by interaction with higher affinity molecular groups, with metabolism etc.

In conclusion, a one-time oral delivery which is releasing a load of above 1 microgram to the pharynx mucosa would provide sufficient protective effect for infection with SARS-CoV-2 for a time period of up to about 30 to 50 minutes depending on local physiological activity. This time horizon is derived from experimental results with sustained release systems.

Example: To an aqueous medium there is added Chitosan to such a concentration that spraying action or taken up medium into the mouth cavity for gargling is providing a single dose of about 1 mg of dissolved Chitosan. There has to be an intense contact to mucosa before the aqueous medium is removed (swallowed or disgorged).

2. In another preferred embodiment, in one-time delivery modus Chitosan solution is applied to the upper mucosa and the respiratory tract by means of inhalation or dropwise application to achieve a therapeutic dose at Pharynx and upper respiratory tract.

Example: An isotonic aqueous biocompatible solution (permitted for application to nose, a nose drop base solution) containing Chitosan is applied by either spraying or dropwise addition. The total dose per application should be limited to 1 mg (0.5 mg per nare).

Example: To a nose oil formulation there are added Chitosan classified microparticles (or complex classified microparticles of a biopolymer combination of Chitosan and Hyaluronic Acid (but not restricted to this combination). This oil suspension is either sprayed of added dropwise to the nose and the concentration of suspended Chitosan was selected such that there are delivered not more than about 1 mg Chitosan to nose (0.5 mg per nare).

This approach could be executed by a nose spray and the release should be adjusted to providing a dose of above 1 microgram Chitosan to nose mucosa and resp. pharynx mucosa. It will result in an efficient protection for at least half an hour.

This approach could also be executed by filling sterilized isotonic inhalation solution loaded with chitosan into one-time usage containers that can be inhaled by usage of ultrasonic inhalation devices.

3. In another preferred embodiment Chitosan is incorporated into resp. beverages, like coffee, tea of any composition, juice, lemonade, milk, cola, alcoholic beverages etc. One should make sure that the dose is between 0.1 and 10 mg Chitosan per sip, preferred between 1 and 5 mg Chitosan per sip. The incorporation can be done at all temperatures from fridge temperature just above 0° C., over ambient to cooking water temperature. Concentrations outside the given range are still active.

Example: To 10 to 30 ml (one sip) of the beverages (tea, coffee, juice, lemonade, milk, cola, alcoholic beverages) there is added 1 mg of Chitosan. Thereafter the tea is taken up in the mouth and moved for 10 to 20 seconds to achieve an intense contact to pharynx mucosa. Gargling would be a preferred activity. After this procedure the beverage could be either swallowed or disgorged.

Example: To 500 g of tea leafs there is added 5 g of Chitosan. To 2 g of this mixture there is added hot water and tea beverage is prepared according to procedures usually taken according to tradition and routine.

4. In another preferred embodiment, in sustained release modus, Chitosan is applied to mucosa of Pharynx by means of release from oral or buccal sustained delivery systems to achieve and maintain a therapeutic dose at Pharynx mucosa.

Example: A layered structure of films (one side sticky to mucosa and the other side controlling release) is doped with Chitosan (preferred about 2 mg, but not restricted to 2 mg) and is fixed to a site in mouth cavity via sticky adhesion. By a combination of release control and dissolution of the film structure the Chitosan is delivered over a time period of minutes and trapped into the several mucosa areals covering the mouth cavity.

5. In another preferred embodiment, oral or buccal sustained delivery systems comprise all kind(s) of solid (Solida) or semisolid (Semisolida) dosage forms which could be, but not restricted to, chewing gums, tablets, capsules, gel bodies, candies, bonbons or oral thin and thick sticky films made of bulk material and/or previously mechanically altered condensed biopolymer material to enhance release times and applied doses.

The protection time of these systems is to be calculated by summing roughly the release time from mentioned depots plus time period of efficient protection of already released but surviving Chitosan above critial concentration as given under 1. or 2.

Example: To a chewing gum raw base of 1.5 g with following qualitative composition there is added 10 mg of a salt-like Chitosan, like Chitosanlactate or Chitosanacetate or Chitosanhydrochloride. Sweetener: Xylitol, Sucralose; Gum Base; Flavourings; Emulsifier: Lecithins. For other Chewing Gum base masses the Chitosan amounts can be adapted.

Example: Similar to Example (1) but a different composition of the Chewing Gum: Sweeteners: Sorbitol, Maltitol Syrup, Isomalt, Sucralose; Gum Base; Humectant: Glycerol; Maltodextrin; Flavorings; Emulsifier: Lecithins. For other Chewing Gum base masses the Chitosan amounts have to be adapted.

Example: To a Chewing Gum Base of 1.5 g, there is added 20 mg of Chitosan (vegan Chitosan of fungi source or Chitosan of animal source, in both cases the Chitosan can be activated by exposing it transiently to an acid pH and/or an elevated temperature or to a combination of both, or, alternatively to release the chitosan in an active form, with, at least, a tendency to form a cationic appearance. For other Chewing Gum base masses the Chitosan amounts can be adapted.

Example: A chewing gum in accordance with a preferred embodiment of the invention comprises: sweeteners: sorbitols, isomalt, maltitols, xylitol, acesulfame K, sucralose; gum base; flavorings; humectant: glycerol; thickener: gum arabic; chitosan; zinc acetate; glazing agent: carnauba wax. Optionally, the chewing gum may comprise citric acid as acidity regulator. Optionally, a chewing gum of 2 g may comprise 20 mg chitosan.

6. In another preferred embodiment, oral or buccal sustained delivery systems comprise solid or semisolid dosage forms which could contain but are not limited to composite microparticles, one component of which is Chitosan.

Chitosan could be one component of a more complex dosage form. For example, Chitosan is forming a complex with Hyaluronic Acid which could be formulated in a sticky microparticle configuration.

7. In another preferred embodiment, the Chitosan dosage is selected to achieve at mucosa target a loading from 1 nanogram per square centimeter to 0.1 microgram per square centimeter, and preferred from 10 nanogram per square centimeter to 0.1 microgram per square centimeter. These doses are preferred but overdose or underdose should still operate satisfying.

These numbers correspond to those given in 1.

The dose taken up by mucosa should be arranged between 10 ng per cm² and 10 microgram per cm², more preferred between 0.1 microgram per cm² and 5 microgram per cm², most preferred between 0.1 microgram per cm² and 1 microgram per cm².

8. In another preferred embodiment, the delivery systems preferably release Chitosan to maintain the mucosa loading at a selected level for a desired period or periods of time.

Example: A sustained Chitosan release could be achieved, for example, by a (refillable) drug delivery implant. There are several implantable devices available which could serve for this purpose, for example, functional dental prosthesis, prosthesis fixation glue material.

9. In another preferred embodiment, Chitosan solutions for spraying solutions or drinking purposes contain essentially:
Dissolved and/or dispersed chitosan and
optionally, aroma ingredients and
optionally, ingredients for modifying taste and appearance and
optionally, ingredients for modifying the rheological properties and
optionally, ingredients for improving the mouth feeling and
optionally, ingredients which are improving the applicability.

10. In another preferred embodiment, the spraying solution for oral and nose applications could contain optionally Hyaluronic Acid or any other permissible biopolymer in stoichiometric or non-stoichiometric ratios to Chitosan.

11. In another preferred embodiment, sustained release spraying forms can include but are not limited to lipophilic/non-chitosan-dissolving liquids with dispersed microparticles of different sizes (for example, 5 μm-950 μm in diameter) of different composition with one ingredient being chitosan. These microparticles can be of a mechanically altered state with comparable composition. An advantage of non-aqueous continuous phase systems is gain in stability of dispersed biopolymers or biopolymer-containing complexes as compared with aqueous systems.

12. In another preferred embodiment, the present invention contemplates oral, buccal and bronchial sustained delivery systems comprising systems of solid co-agglomerates of composites of different microparticles with one of the ingredients being Chitosan. Larger particles will impact in the upper respiratory tract while particles of 5 μm diameter and smaller will impact in the bronchi delivering Chitosan over a longer period of time.

Involved microparticles can consist solely out of chitosan and its water soluble salts or of combinations of chitosan with other biopolymers forming complex composite particles.

This build-up strategy of complex composite particles is covering several orders of magnitude. Fluid Bed Granulation, inter alia, comprises one set of techniques to fabricate this family of complex particles.

Example: A fluid bed granulation equipment is feeded with hyaluronic acid microparticles. To the fluid bed there is added (sprayed to) a solution of Chitosan. This process is continued up to achieving the target microparticle aggregate size. The process could be continued with the now obtained microparticle aggregate as starting particle, etc.

13. In another preferred embodiment the Chitosan or Chitosan-containing dosage forms are microparticles or classified microparticles. The microparticle fabrication is achieved by transforming the biopolymer powder into macroscopic solid bodies and subsequent micronization and classification.

14. In another preferred embodiment, the present invention contemplates chitosan solutions or suspensions for spraying onto technical or household surfaces or into restricted volumes could contain beside solid or dissolved chitosan itself other ingredients, like alcohols (for example, ethanol, propanol), disinfectants and skin-caring ingredients.

Usually biopolymers possess limited stability in aqueous systems. They are degraded by all types of hydrolysis, are losing molecular mass and integrity. To overcome this effect it is proposed to integrate Chitosan in a complex composite structure which provides stability up to usage. It could be achieved by a wide spectrum of stoichiometric ratio between Chitosan and accompanying components.

15. In another preferred embodiment microparticles of different sizes of chitosan with/without other biopolymers can be dispersed in soap bars, liquid soaps and lotions to ensure inactivation of viral contaminations on the skin and avoid smear infections.

16. In another preferred embodiment microparticles of Chitosan or complexes of chitosan with other ingrediences are incorporotated into laundry detergents formulation. They will support antimicrobial effects.

17. In another preferred embodiment Chitosan is incorporated into semisolid gels for application to oral cavity. The released Chitosan from these sticky gel systems is supporting mouth hygiene and anti-gingivitis and paradontosis/paradonditis syndromes.

18. In another preferred embodiment microparticles of different sizes of chitosan can be dispersed in solid salt mixtures to enable simple and easy home manufacturing of nasal irrigation solutions that can also be applied in the oral cavity. Through this way tap water can be turned into Chitosan formulations that can inactivate viral transfection in the upper respiratory tract.

### Example 1: Measurement of Chitosan Release from Chewing Gum

The inventors evaluated the effect of chewing of two chewing gum products (236.03 SPF1490 and 236.02 SPF1494) containing 10 mg chitosan. The concentration of chitosan in the saliva by having 4 volunteers to chew these two sugarless gum products has been measured. The intention has been to explore the time dependent mechanisms of secretion of chitosan.

Saliva samples have been collected before the start of chewing and 1, 2, 3, 4, 5, 5-10 very minute collected, additional 15 min, then collected and then the gum was removed. After 15 min and 30 min without a gum in the mouth, the saliva has again been collected and measured. Volunteers chewed 30-60 times every 1 min.

Chitosan has been determined by measurement with Hettich solution. In brief, this has been conducted according the following steps. Nunc Maxisorb 96 well plates were filled with 180 μl buffer pH 6.6 (gentle Ag/Ab elution buffer Prod #21027 from Therom scientific). 10 mg of chitosan salt was dissolved in puffer pH 6.6 (gentle Ag/Ab elution buffer Prod #21027 from Therom scientific). Standard curves were generated with serial dilutions from 0.9 mg/ml, 0.45 mg/ml, 0.3 mg/ml, 0.15 mg/ml, 0.075 mg/ml; 0.0352 mg/ml (chitosan in PBS or saliva) addition of 5 μl Hettich solution (2.5 g Iodide, 2.5 g of KI in 90% of ethanol), reaction was measured at wavelength 620 nm. 10 μl of each sample was added and 5 μl of Hettich solution (2.5 g Iodide, 2.5 g of KI in 90% of ethanol) was added. After 10 minutes, reaction was measured at wavelength 620 nm. Concentrations were calculated according to standard curve measurement.

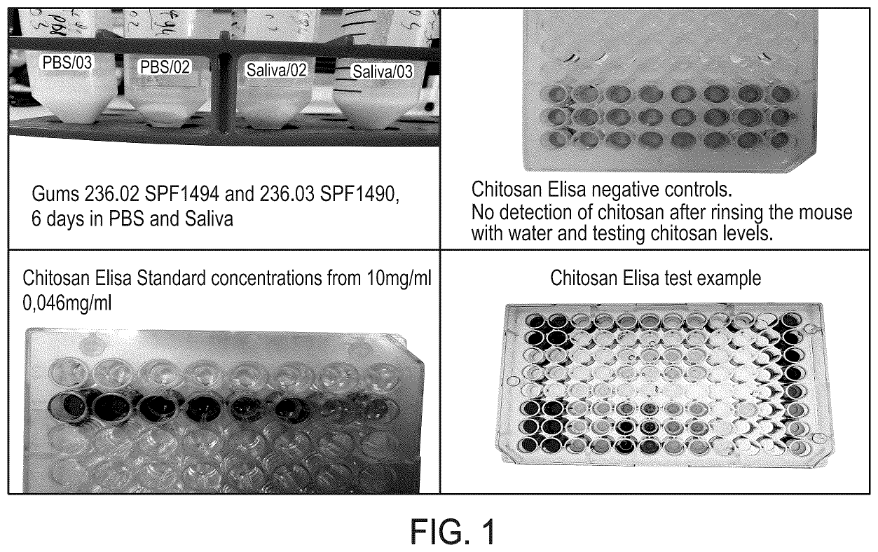

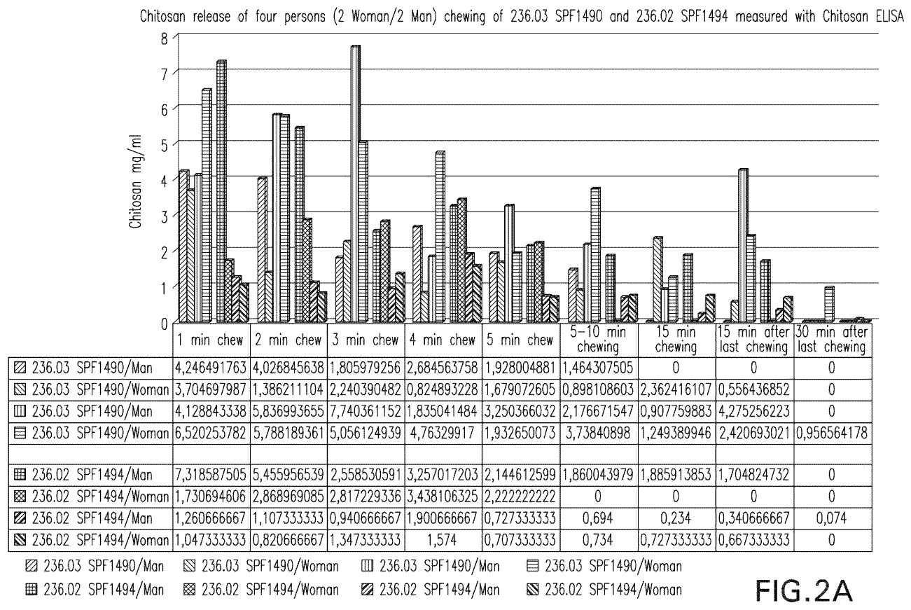

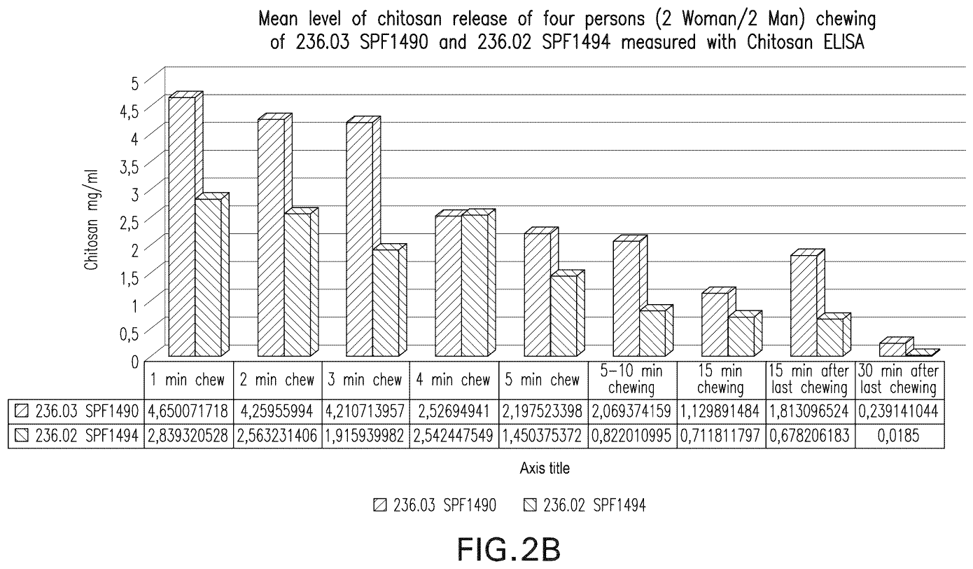

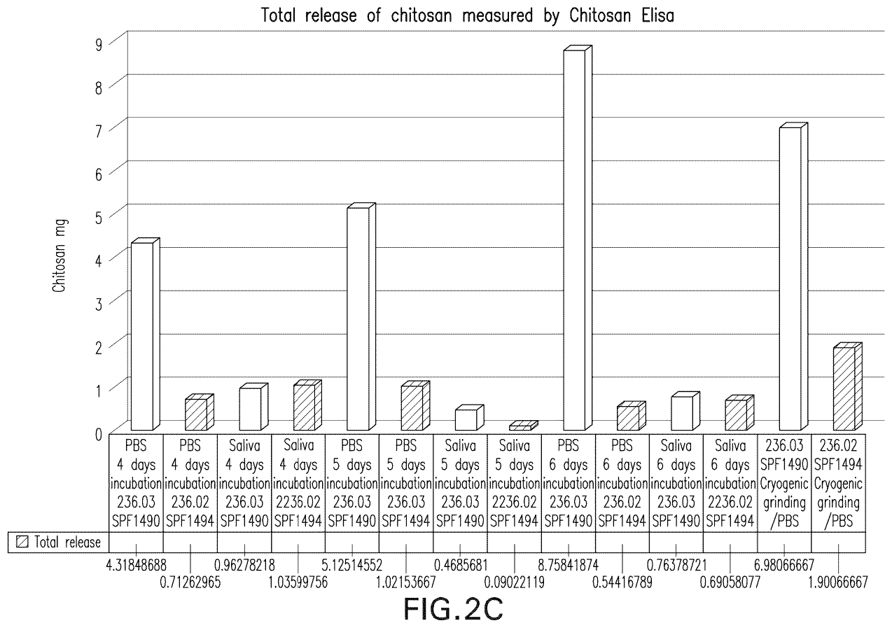

The mean chitosan level in the first minute of chewing for 236.03 SPF1490 is 4.6 mg (mean value) and its release after 6 days (see FIG. 1, and FIGS. 2A to C) in PBS is 8.7 mg. This is 12.4 times higher than the release to be measured after 6 days in the saliva without mechanical stress of chewing. From these data it can be concluded that mechanical stress of chewing enhances the release >10× and that in the saliva the chitosan is degraded constantly even at 4° C., the temperature at which the samples were stored. When using 236.03 SPF1490, 70% of the chitosan is released 3 minutes after cryogenic grinding in liquid Nitrogen in 5 ml of PBS. When using 236.02 SPF1494 20% of the total chitosan is released also very quickly in 3 minutes after cryogenic grinding in liquid nitrogen in 5 ml of PBS.

The mean chitosan level in the first minute of chewing of 236.02 SPF1494 is 2.8 mg per average and its release after 6 days in PBS is 0.5 mg. This is 5.6 times higher than the release to be measured after 6 days in the saliva without mechanical stress of chewing This is 16.1 times lesser than the release of 236.03 SPF1490 to be measured after 6 days in the PBS.

Interestingly, the release of 236.03 SPF1490 and 236.02 SPF1494 of chitosan in the saliva after 6 days is nearly the same (0.76 mg; 0.69 mg).

From these data it can be concluded that chitosan is released in the saliva from the two different gum products without mechanical stress and degraded in the same manner in both gums tested. In the first 3 minutes the average release is constantly high (— 2-5 mg) and in the 4th minute both gums release the same 2.5 mg/ml on average in the 4 persons tested.

The chitosan level drops than from 2.5 after 5 min to 1.4 mg in the 236.02 SPF1494 in the next 5-10 min and another 15 min of chewing it drops to 0.8-0.7 mg/ml chitosan release. The chitosan level drops than from 2.5 after 5 min to 2.1 mg in the 236.03 SPF1490 in the next 5-10 min and another 15 min of chewing it drops to 1.1-1.8 mg/ml chitosan release.

As a next step, the inventors tested the chitosan level 15 minutes after chewing in the mouth saliva. No eating and drinking have been allowed. The inventors were still able to measure by average of 1.8 mg/ml chitosan from 236.03 SPF1490 and 0.7 mg/ml from 236.02 SPF1494 after 15 minutes and 0.23 mg/ml of 236.03 SPF1490 and 0.0185 mg/ml chitosan from 236.02 SPF1494 in the saliva after last chewing event.

This means that protection of infection is possible in the time of chewing and even 15 to 30 min after the release of the gum from the mouth.

In view of the above, the inventors have experimentally demonstrated in a series of experiments that chitosan is efficiently released from the chewing gum during the chewing process. Further, they could experimentally demonstrate that there is chitosan detectable in the oral cavity after and even 20 minutes beyond finishing the chewing process and removing the chewing gum.

### Example 2: Microscale Thermophoresis (MST) Binding Studies

Starting from the available structural data on the interaction between SARS-CoV-2 Spike protein and the host ACE2 receptor, the inventors have engineered a RBD region with the aim of creating a soluble viral receptor and stable ACE2 interactor. This mini-RBD-SARS-CoV-2 Spike protein (around 90aa), which was recombinantly produced in high yields, possesses a stable conformation and is able to interact with the ACE2.

Microscale thermophoresis (MST) analysis was performed to measure the binding affinity of Mini RBD S protein of SARS-CoV-2 to human ACE2.

The thermophoretic measurements were performed using Monolith NT.115 device (NanoTemper Technologies, Munich, Germany). Chitosan was bought from a company Kraeber. For MST recording, the recombinant proteins were labelled with the fluorescent dye Cy-5 (SNAP-tag Kit Red nanotemper). Thermophoretic experiments were conducted using Monolith NT.115 (NanoTemper Technologies, Munich, Germany). Recombinant proteins concentration after labelling was measured using a UV-Vis spectrophotometer and the labelling efficiency was determined to be 80%. The MST experiment was performed in a buffer containing PBS, pH 7.5. 10 μL of the RBS-Snap-Tag protein 10/20 nM was mixed with 10 μL of 16 serial dilution of chitosan and other sugars. The final concentration of S protein was 20 nM in all samples, whereas chitosan, L+Arabinose, D+Galactose, Nactely-D-glucosamine, Coffein concentration ranged from 0.5 μM to 0.2 nM. Samples were then loaded into sixteen premium-coated capillaries (NanoTemper Technologies) and fluorescence was recorded for 20 s using 100% laser power and 40% MST power. The temperature of the instrument was set to 25° C. for all measurements. After recording the MST time traces, data were analysed. KD value was calculated from ligand concentration-dependent changes in the normalised fluorescence of the rec protein after 14 s of thermophoresis. The assay was performed in triplicates and the values reported were generated through the usage of MO Affinity Analysis software (NanoTemper Technologies).

Affinity parameters for chitosan, L+Arabinose, D+Galactose, N-actely-D-glucosamine, Coffein binding to Snap tag RBD.

| | Soluble Snap tagged RBD-Cy5 |
|---|---|
| Substance | kD (nM) |
| Chitosan (20 kd-460 kD) | 6-138 nM |
| D + Galactose | 355-381 nM |
| N-actely-D-glucosamine | 176-194 nM |
| Coffein | 55-71 nM |
| L + Arabinose | no binding |

There are two data sets, with the ranges generated from 3 experiments to determine the initial binding 6 experiments where each set of data within one experiment with a general fit using a 1:1 binding model. Average and standard from binding curve fit; range representative of six experiments.

MST measurements show nanomolar binding affinity of Chitosan; D Galactose, D Mannose and caffeine binding to mini RBD of SARS-Cov2.

No binding was observed with L+Arabinose and N actely-D-glucosamine.

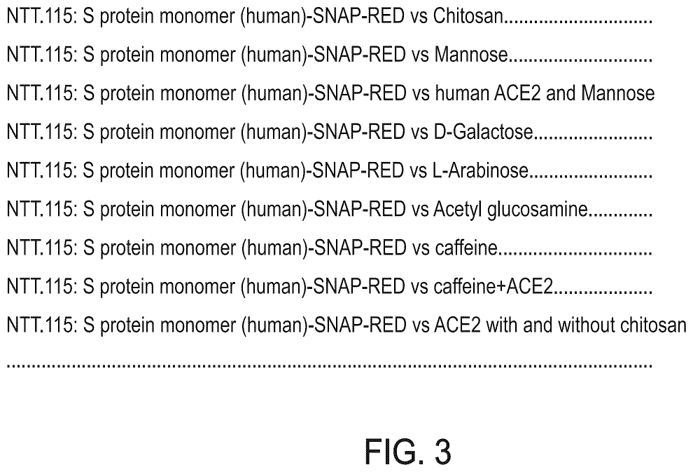

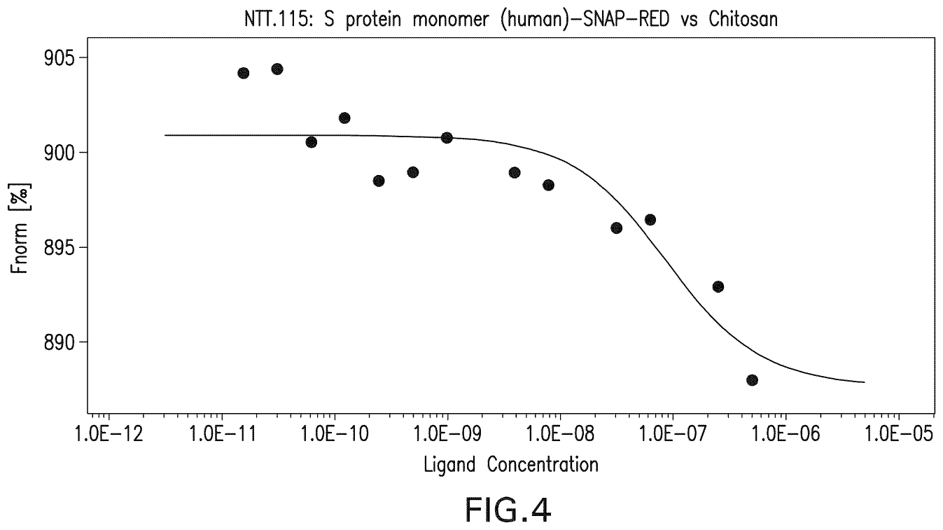

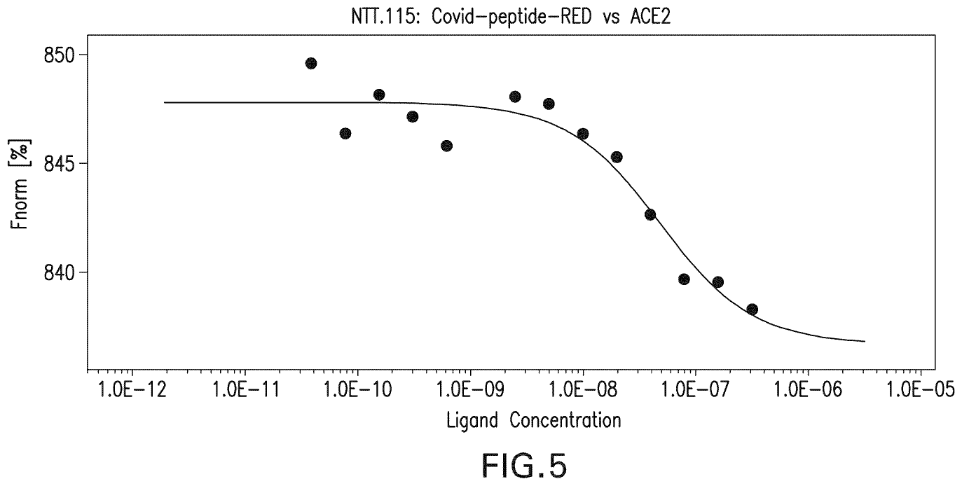

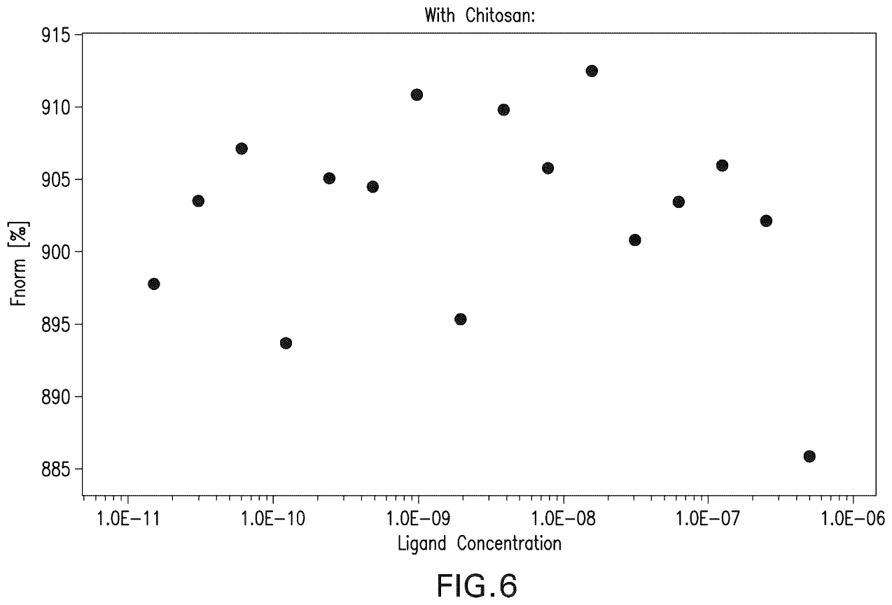

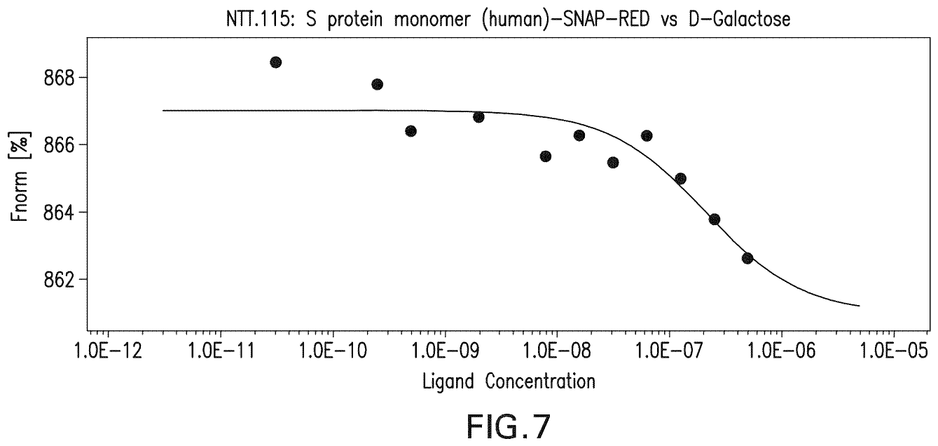

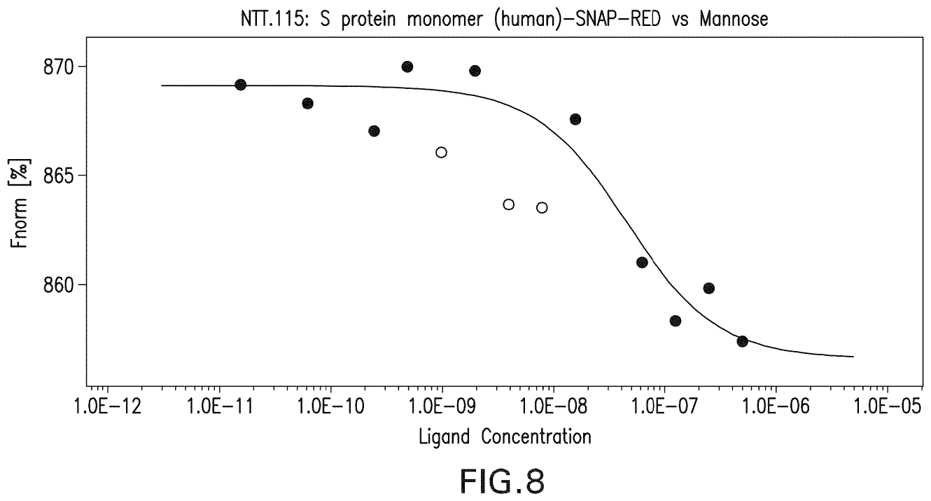

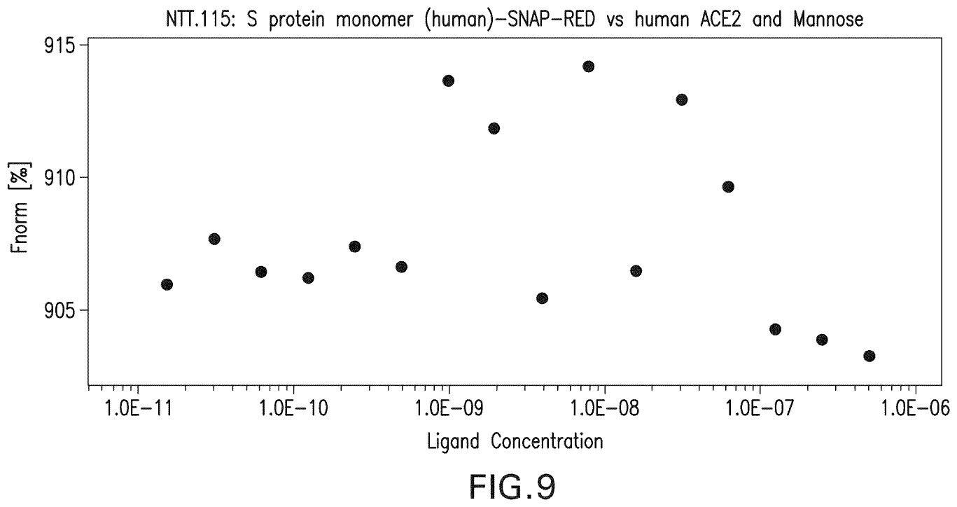

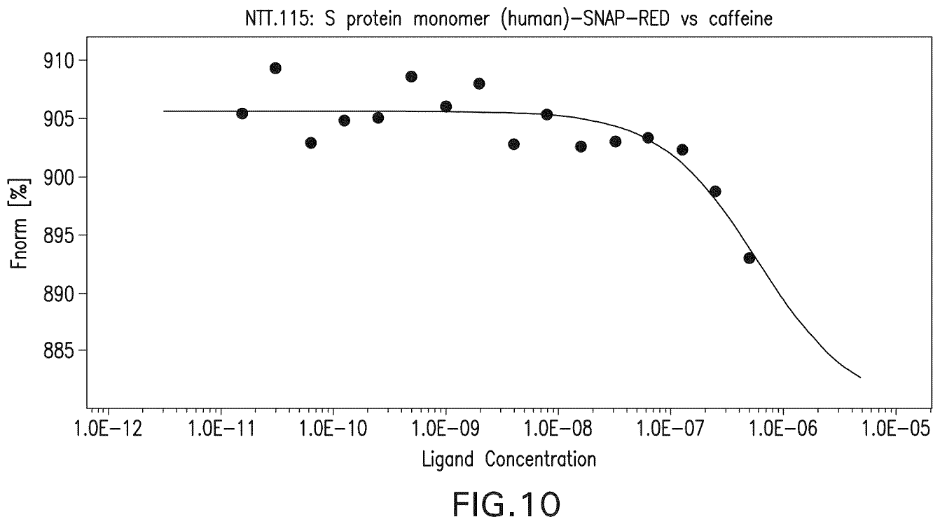

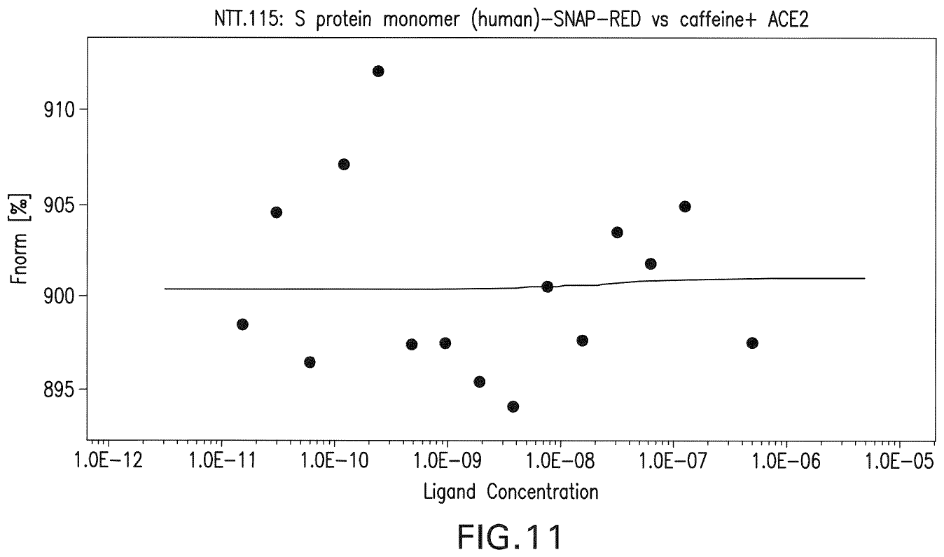

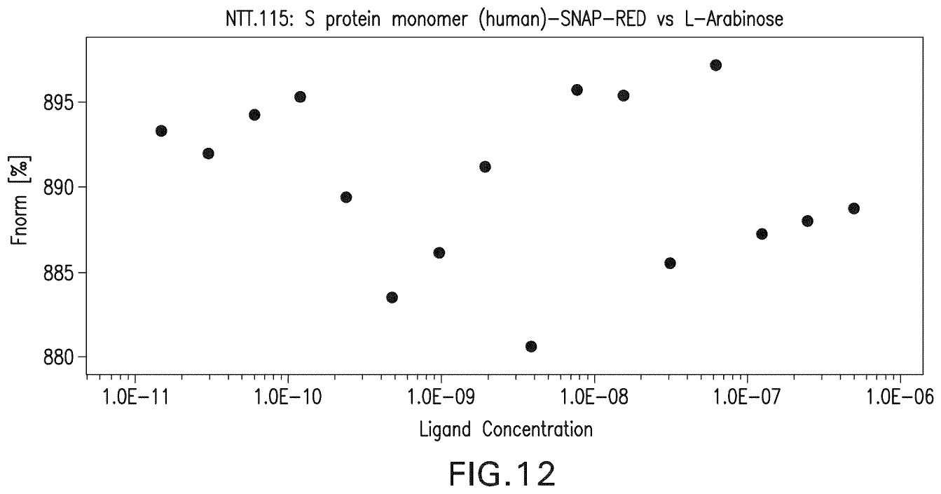

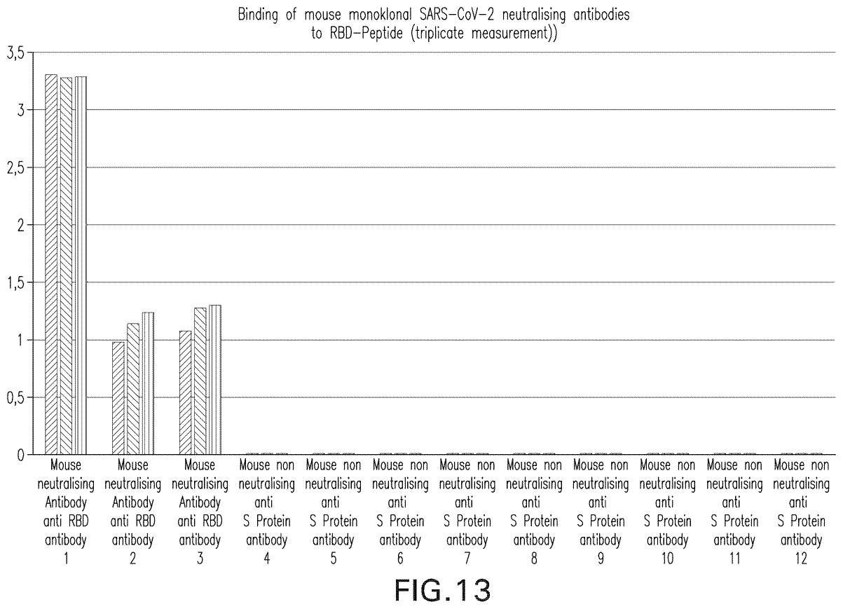

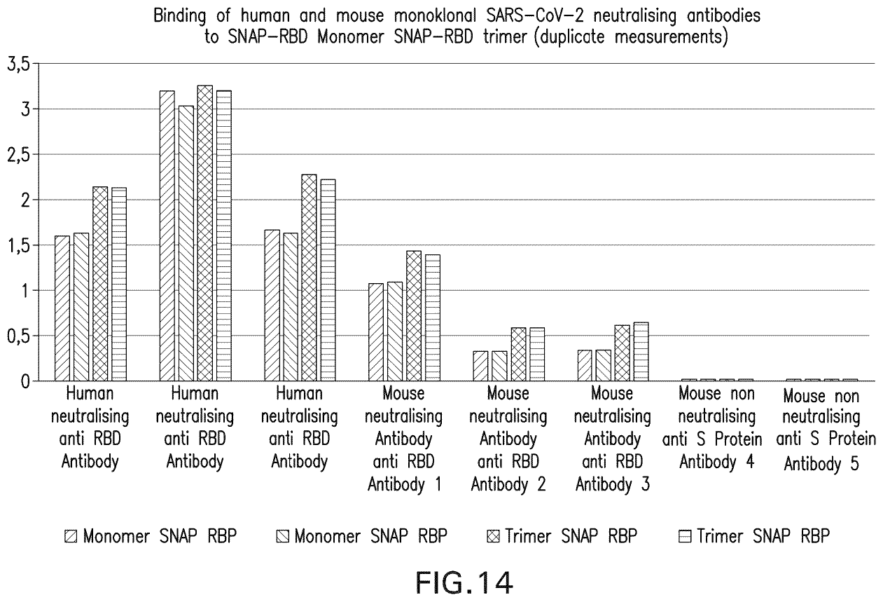

Moreover Chitosan, D Galactose, D, Mannose and Caffeine could inhibit the binding of the mini-RBD of Cov19 with the human ACE2 receptor. The results are shown in FIGS. 3 to 12.

### Example 3: ELISA Binding Assay Testing the Binding to SARS-COV-02 N-Protein Tested with Different COVID-19 Patient Sera Via Chitosan Lactate

To determine the binding, the inventors used an ELISA assay. For conducting the ELISA assay, a procedure with the following steps has been conducted by the inventors. The inventors have used a Proteintech® ELISA RBD Covid IgG ELISA kit.

S-RBD peptide coupled to an ELISA plate has been incubated with buffer without chitosan as control and with chitosan, 10 mg/ml lactate, 1 mg/well in buffer for 1 h at RT on a shaker at 350 rpm with 100 μl/well. Then, a washing step was conducted with 5 times washing with a wash buffer with 400 μl/well. Afterwards, serum of COVID-19 infected patients in a 1:100 in serum dilution buffer has been incubated for 1 h at RT on a shaker at 350 rpm with 100 μl/well.

Then, a further washing step has been performed with 3 times washing with a wash buffer with 400 μl/well. As a next step, the detection antibody in dilution buffer has been incubated for 30 minutes at RT on a shaker at 350 rpm with 100 μl/well. A further washing step has been performed with washing 3 times with wash buffer with 400 μl/well. A developing solution has been incubated for 10 minutes at RT with 100 μl/well. Then 100 μl/well of a stop solution has been added. Finally, the wells have been analysed in a spectrometer with a read-out at 450-620 nm.

The results of the binding ELISA are shown in FIG. 15.

FIG. 15A shows the median elevation in form of the change in percent of binding enhancement of serum antibodies concerning the binding to SARS-COV-02 RBD-protein in comparison to saliva control without chitosan. Three groups of antibodies of COVID-19 infected patients have been tested: high RBD SARS-COV-02 binding serum antibodies, medium RBD SARS-COV-02 binding serum antibodies, and low RBD SARS-COV-02 serum antibodies. For each group of antibodies three dilutions have been tested, namely 1:100, 1:200, and 1:400 dilution in saliva. According to FIG. 15A in particular low binding neutralising serum antibodies show an elevated binding in comparison to the control without chitosan.

FIG. 15 B shows the median elevation in form of the change in percent of binding enhancement of serum antibodies concerning the binding to SARS-COV-02 RBD-protein in comparison to saliva control without chitosan. Again chitosan as released from chewing gum samples have been used for the first incubation step in the ELISA assay. According to FIG. 15B in particular low binding neutralising serum antibodies show a slightly elevated binding in comparison to the control without chitosan.

A similar ELISA assay as shown in FIGS. 15A and B has been also conducted for the nucleocapsid protein of SARS-CoV-2 with different Covid-19 sera in the presence of chitosan—chitosan lactate and chitosan in saliva. Increased binding of neutralising antibodies specific for the nucleocapsid could be observed as shown in FIGS. 17A, and 17 B

### Example 4: Anti-SARS-CoV-2 S Protein (RBD) Human IgG ELISA Kit Chitosan Modification

The objective of the study was to develop and validate the (RBD) human IgG ELISA Kit method further for the detection of specific antibodies to SARS-COV-02 and provide a tool for the diagnosis and the future estimation of the performance.

According to the result of current study, the clinical sensitivity and specificity from all detecting systems does not meet the requirements of screening, diagnosis and confirm diagnosis experiments. Therefore, the ELISA systems tested cannot be used for the diagnosis and of SARS-CoV-2 infections. False negative and false positive results will appear in the detection, which will cause confusion to clinical judgment. So the company needs to pay close attention to the performance indicators of the reagents used.

In the present study, the inventors modified the RBD ELISA method from a known ELISA method in four complementary modified formats. The conventional ELISA format for general laboratory usage was used as control. With easy to perform modified procedures and buffers the inventors wanted to enhance the accuracy of the test.

The inventors showed that all new formats performed better than the standard, and their results correlated nicely when modified. When comparing by ROC analysis, the new ELISA SOPs methods with both formats was shown to have highest possible accuracy.

In the current study, the inventors applied ELISA for the analysis of 15 Covid 19 samples and 11 ANA control samples. The standard ELISA is analysed using the same serum samples. ROC has been used to calculate the optimal diagnostic threshold (Cut off value) of the detection system. Investigation has shown that the clinical specificity and sensitivity 2019-nCoV IgG n=15) antibody are 100% when 0.15 OD is used respectively. Therefore, the false negative and false positive results can be eliminated in the detection, which will cause excellent clinical judgment (see FIG. 26).

FIG. 26 shows the result of the modification of the anti-SARS-CoV2-S-RBD ELISA assay. Modifications 1 and 2 of rows 1 and 2 included the addition of 1 mg/mL chitosan lactate with an incubation time of 30 minutes in a serum dilution of 1:200. Row 3 represented the standard assay without chitosan. Modification 1 and 2 of rows 3 and 4 included the addition of 1 mg/mL chitosan lactate with an incubation time of 60 minutes in a serum dilution of 1:100

In conclusion, it is possible to developed a high-throughput, reliable and specific ELISA method to study humoral immune responses in Covid 19 patients. With this method, it will be possible to perform the serodiagnosis of SARS-Cov-2 infections and to assess the seroprevalence within human populations in the future.

SARS-CoV-2 infection can also be indirectly detected by measuring the immune response of the infected person to the virus. Serological diagnosis is particularly important for patients with a low-symptomatic disease. In addition, serological diagnosis also becomes an important tool to understand the extent of Covid 19 disease in a given population and to identify individuals who are potentially “protected” from infection. The inventors believe that with a modified SOP S-RBD ELISA IgG antibody tests may reach over 95% specificity for the diagnosis of Covid-19.

Testing paired serum samples first with RT-PCR and two weeks later with the N protein ELISA test can further increase diagnostic accuracy. In general, the majority of antibodies formed against the most common nucleocapsid protein (NC) of the virus. Therefore, tests that detect NC antibodies are considered the most sensitive. More informative, however, is the detection of antibodies against the receptor binding domain of the spike protein (RBD-S), as there is a higher probability that neutralizing antibodies are also found among these antibodies. It should be noted that some of the tests on the market cross-react with antibodies against SARS-CoV-1. This can be analysed as well with the two known ELISAs in a further SOP-IVD development program.

The present invention can be also described as follows:

Chitosan is comprising Polyglucosamines of many different molecular weights, and many different degrees of de-acetylation as well as all biocompatible derivatives thereof and types of crosslinked derivatives and physical modifications. Preferably, chitosan has to be applied in cationic form or has to acquire cationic properties at mucosa site.

Furthermore, Chitosan is understood as an example for chemically synthesized and extracted organic and inorganic polycationic biopolymers and polymers that can be respectively used. Other examples comprise but are not limited to polyethylenimine, etc.

In a preferred embodiment, it is contemplated that the proposed emergency application of the Chitosan carbon hydrate for antiviral purposes (COVID-19 (Coronavirus SARS-CoV-2) could trigger the development of a biotechnological fabrication route, as could be observed for insulin, hyaluronic acid and so many antibodies. So far, Chitosan is made from animals or fungi.

The polysaccharide Chitosan has excellent biological properties, for example biocompatibility, support of wound tissue regeneration, immunostimulation, induction of hemostasis and radical scavenging as well as antimicrobial and antiviral activities.

Chitosan has many applications in areas as diverse as the cosmetic industry, medical industry, agricultural industry, food processing, nutritional enhancement, and, wastewater treatment

The present invention contemplates many embodiments described herein.

Chitosan can be used for drastic reduction of virus transfection of several biological tissues.

Chitosan is a candidate for drug and gene delivery systems, a key component in diagnostic test protocols and provides anti-adhesion property and acts as a component in surgical devices.

Chitosan is an active and efficient ingredient of anti-itching formulations.

Chitosan is a nutritional supplement controlling fat resorption in the gastrointestinal tract.

There are viruses which are penetrating mucosa and transfect subjacent biological cells.

The virus Coronavirus SARS-CoV-2 attacks the mucosa and subjacent cells of human pharynx. The human pharynx is accessible via oral cavity, nose and throat tract. The nose itself is accessible from the eyes via lacrymal duct as well as from the ear via eustachian tube.

In a preferred embodiment, the present invention contemplates that, in a controlled and sustained manner, Chitosan and mucosa of Pharynx could inhibit transfection of Coronavirus SARS-CoV-2 to a degree strong enough to prevent or reduce infection or to promote immunization without pronounced disease.

It is contemplated that chitosan interacts with viruses or virus bodies. One type of interaction is of an electrostatic character. Same type is observed in interaction between Chitosan and cell surface. It is assumed there exists, for each Chitosan molecule, more than just one interaction site with the membrane. Two and more interaction sites at a distance would disturb intrinsic membrane mechanics and, as consequence, affect also virus cell membrane interaction. Ultimately, it is contemplated that by a chain of events the virus transfection probability is reduced up to a factor of hundred.

Because of interactions of the Chitosan with viruses (if available), mucosa components (mainly of negative electric charge) and the adjacent cell membrane surface, its amount or concentration has to be tuned correctly to match with the therapeutic window. The delivery of Chitosan to Pharynx mucosa, either by one-time or sustained release, has to be adjusted to that therapeutic range. This range itself is not known a priori and will be determined experimentally for each pathogen.

The present patent application also contemplates procedures to deliver a therapeutic chitosan dose to the Pharynx mucosa, preferably in a one-time or in a sustained release manner, to get a therapeutic dose for a sufficient period of time.

One-time application can be achieved, for example, by spraying, gargling or slow drinking in small sips of diluted Chitosan solutions or suspensions. Inhalation of Chitosan-containing aerosols is a procedure that can preferably be used to deliver to numerous targets, including Pharynx, respiratory tract and nose. Spraying and inhalation procedures are executed by means of state-of-the-art devices.

The present patent application also contemplates that spraying of Chitosan solution or suspensions onto surfaces of living, hospital or working rooms causes or produces anti-viral and anti-bacterial effects. The same would work by just spraying into the room or indoor air.

Here are several representative, non-limiting examples:

1. In one preferred embodiment, in one-time delivery modus Chitosan solution is applied to mucosa of Pharynx by means of spraying or gargling to achieve a therapeutic dose at Pharynx mucosa.

2. In another preferred embodiment, in one-time delivery modus Chitosan solution is applied to the respiratory tract by means of inhalation to achieve a therapeutic dose at Pharynx and upper respiratory tract.

3. In another preferred embodiment, in sustained release modus Chitosan is applied to mucosa of Pharynx by means of release from oral or buccal sustained delivery systems to achieve and maintain a therapeutic dose at Pharynx mucosa.

4. In another preferred embodiment, oral or buccal sustained delivery systems comprise all kind(s) of solid (Solida) or semisolid (Semisolida) dosage forms which could be, but not restricted to, chewing gums, tablets, capsules, gel bodies or oral thin films made of bulk material and/or previously mechanically altered condensed biopolymer material to enhance release times and applied doses.

5. In another preferred embodiment, oral or buccal sustained delivery systems comprise solid or semisolid dosage forms which could contain but are not limited to composite microparticles, one component of which is Chitosan.

6. In another preferred embodiment, the Chitosan dosage is selected to achieve at mucosa target a loading from 1 nanogram per square centimeter to 0.1 microgram per square centimeter, and preferred from 10 nanogram per square centimeter to 0.1 microgram per square centimeter.

7. In another preferred embodiment, the delivery systems preferably release Chitosan to maintain the mucosa loading at a selected level for a desired period or periods of time.

8. In another preferred embodiment, Chitosan solutions for spraying solutions or drinking purposes contain essentially,
Dissolved and/or dispersed chitosan and
optionally, aroma ingredients and
optionally, ingredients for modifying taste and appearance and
optionally, ingredients for modifying the rheological properties and
optionally, ingredients for improving the mouth feeling and
optionally, ingredients which are improving the applicability.

9. In another preferred embodiment, the spraying solution for oral and nose applications could contain optionally Hyaluronic Acid in non-stoichiometric ratio to Chitosan.

10. In another preferred embodiment, sustained release spraying forms can include but are not limited to lipophilic/non-chitosan-dissolving liquids with dispersed microparticles of different sizes (for example, 5 μm-950 μm in diameter) of different composition with one ingredient being chitosan. These microparticles can be of a mechanically altered state with comparable composition.

11. In another preferred embodiment, the present invention contemplates oral, buccal and bronchial sustained delivery systems comprising systems of solid co-agglomerates of composites of different microparticles with one of the ingredients being Chitosan. Larger particles will impact in the upper respiratory tract while particles of 5 μm diameter and smaller will impact in the bronchi delivering Chitosan over a longer period of time.

12. In another preferred embodiment, the present invention contemplates chitosan solutions or suspensions for spraying onto technical or household surfaces or into restricted volumes could contain beside solid or dissolved chitosan itself other ingredients, like alcohols (for example, ethanol, propanol), disinfectives and skin-caring ingredients.

The present invention is also characterized by the following items:

1. A solid composition for use in preventing an infection by a virus or bacterium, or treating a virus or bacterial infection, or treating a disease caused by a virus or bacterial infection, the composition comprising a compound selected from the group consisting of chitosan or a salt thereof, galactose, mannose, caffeine, and any combination thereof.

2. The composition for use of item 1, wherein the virus is a respiratory virus.

3. The composition for use of item 2, wherein the respiratory virus is selected from the group consisting of a coronavirus, preferably a human-pathogenic coronavirus, an influenza virus, a rhinovirus, and a respiratory syncytial virus.

4. The composition for use of item 3, wherein the human-pathogenic coronavirus is a betacoronavirus.

5. The composition for use of item 3 or 4, wherein the human-pathogenic coronavirus is selected from the group consisting of SARS-CoV-2, MERS-CoV and SARS-CoV-1, preferably wherein the human-pathogenic coronavirus is SARS-CoV-2.

6. The composition for use of item 1, wherein the disease is selected from the group consisting of COVID-19, MERS and SARS, preferably wherein the disease is COVID-19.

7. The composition for use of any one of the preceding items, wherein the use comprises contacting a bodily fluid with the composition.

8. The composition for use of item 7, wherein the bodily fluid is saliva or a mucous secretion.

9. The composition for use of item 8, wherein the mucous secretion is a nasal secretion or a bronchial secretion.

10. The composition for use of any one of the preceding items, wherein the use comprises contacting a mucosa with the composition.

11. The composition for use of item 10, wherein the mucosa is selected from the group consisting of mucosa of the nose, mucosa of the pharynx, mucosa of the mouth and mucosa of the bronchia.

12. The composition for use of any one of the preceding items, wherein the compound is chitosan or a salt thereof.

13. The composition for use of any one of the preceding items, wherein the salt is selected from the group consisting of chitosan lactate, chitosan acetate, chitosan hydrochloride, and any combination thereof.

14. The composition for use of any one of the preceding items, wherein the composition is an edible composition or a composition suitable for oral, nasal or bronchial administration.

15. The composition for use of any one of the preceding items, wherein the composition is a dietary supplement, preferably wherein the dietary supplement is suitable for being added to a beverage, more preferably wherein the beverage is selected from the group consisting of tea, coffee, juice, lemonade, milk, cola, a drinkable milk product (e.g. drinkable yoghurt), a beverage obtainable by dissolving a sherbet powder or an effervescent tablet, and an alcoholic beverage.

16. The composition according to any one of the preceding items, wherein the composition is selected from the group consisting of a chewing gum, a candy, a bonbon, a fruit gum, a chocolate, and a composition for preparing a beverage (e.g. a coffee composition, a tea composition, a sherbet powder, an effervescent tablet).

17. The composition for use of item 16, wherein the composition is a chewing gum.

18. The composition for use of item 17, wherein the chewing gum comprises chitosan and an acid.

19. The composition for use of item 17, wherein the chewing gum comprises a salt of chitosan.

20. The composition for use of item 19, wherein the salt of chitosan is selected from the group consisting of chitosan lactate, chitosan acetate and chitosan hydrochloride.

21. The composition for use of any one of the preceding items, wherein the composition is a sustained delivery system, preferably an oral or buccal sustained delivery system.

22. The composition for use of any one of the preceding items, wherein the composition comprises a solid or semi-solid dosage form.

23. The composition for use of item 21 or 22, wherein the composition is selected from the group consisting of a film, a layered structure of films, a tablet, a capsule, a gel body (e.g. a gel for oral cavity), a chewing gum, a candy, a bonbon, a fruit gum, and a chocolate.

24. The composition for use of any one of the preceding items, wherein the composition comprises a microparticle.

25. The composition for use of item 24, wherein the microparticle comprises chitosan or a salt thereof.

26. The composition for use of item 25, wherein the microparticle further comprises hyaluronic acid.

27. The composition for use of any one of items 24 to 26, wherein the composition comprises a co-agglomerate of different microparticles.

28. The composition for use of item 27, wherein the co-agglomerate comprises chitosan or a salt thereof.

29. The composition for use of any one of items 24 to 28, wherein the microparticle or the co-agglomerate is comprised in a liquid or semi-solid composition, in particular wherein the microparticle or the co-agglomerate is suspended in a liquid or semi-solid composition.

30. The composition for use of item 29, wherein the microparticle or the co-agglomerate is comprised, in particular suspended, in a composition for spraying or gargling, a mouth rinse, a gel for application to the oral cavity, a nose spray, a nose oil, a nose drop formulation, and a nasal irrigation formulation.

31. The composition for use of any one of items 21 to 28, wherein the delivery system comprises a drug delivery implant.

32. The composition for use of any one of the preceding items, wherein the composition is comprised in a mixture for manufacturing a nasal irrigation solution, a nose drop solution, a nose oil formulation, a solution for spraying or gargling, a mouth rinse, a gel for application to the oral cavity, or a beverage.

33. The composition for use of item 32, wherein the composition is in form of a microparticle.

34. The composition of any one of the preceding items, wherein the chitosan or the salt thereof has a degree of deacetylation of 80% or more.

35. The composition of any one of items 1 to 33, wherein the chitosan or the salt thereof has a degree of deacetylation of from 40 to 50%.

36. Non-medical use of a solid composition for preventing an infection by a virus or bacterium, wherein the solid composition is as defined in any one of the preceding items.

37. The use of item 36, wherein the virus is a respiratory virus.

38. The use of item 37, wherein the virus is selected from the group consisting of a coronavirus, preferably a human-pathogenic coronavirus, an influenza virus, a rhinovirus, and a respiratory syncytial virus [RSV].

39. The use of item 38, wherein the human-pathogenic coronavirus is a betacoronavirus.

40. The use of item 38 or 39, wherein the human-pathogenic coronavirus is selected from the group consisting of SARS-CoV-2, MERS-CoV and SARS-CoV-1, preferably wherein the human-pathogenic coronavirus is SARS-CoV-2.

41. The use of any one of items 36 to 40, wherein the use comprises contacting a bodily fluid with the composition.

42. The use of item 41, wherein the bodily fluid is saliva or a mucous secretion.

43. The use of item 41, wherein the mucous secretion is a nasal secretion or a bronchial secretion.

44. The use of any one of items 36 to 43, wherein the use comprises contacting a mucosa with the composition.

45. The use of item 44, wherein the mucosa is selected from the group consisting of mucosa of the nose, mucosa of the pharynx, mucosa of the mouth and mucosa of the bronchia.

46. A method of preventing an infection by a virus or bacterium, or treating a virus or bacterial infection, or treating a disease caused by a virus or bacterial infection by the use of a solid composition, wherein the solid composition is as defined in any one of items 1 to 35.

47. The method of item 46, wherein the virus is a respiratory virus.

48. The method of item 47, wherein the respiratory virus is selected from the group consisting of a coronavirus, preferably a human-pathogenic coronavirus, an influenza virus, a rhinovirus, and a respiratory syncytial virus [RSV].

49. The method of item 48, wherein the human-pathogenic coronavirus is a betacoronavirus.

50. The method of item 48 or 49, wherein the human-pathogenic coronavirus is selected from the group consisting of SARS-CoV-2, MERS-CoV and SARS-CoV-1, preferably wherein the human-pathogenic coronavirus is SARS-CoV-2.

51. The method of item 46, wherein the disease is selected from the group consisting of COVID-19, MERS and SARS, preferably wherein the disease is COVID-19.

52. The method of any one of items 46 to 51, wherein the method comprises contacting a bodily fluid with the composition.

53. The method of item 52, wherein the bodily fluid is saliva or a mucous secretion.

54. The method of item 53, wherein the mucous secretion is a nasal secretion or a bronchial secretion.

55. The method of any one of items 46 to 54, wherein the use comprises contacting a mucosa with the composition.

56. The method of item 55, wherein the mucosa is selected from the group consisting of mucosa of the nose, mucosa of the pharynx, mucosa of the mouth and mucosa of the bronchia.

57. A chewing gum comprising a compound selected from the group consisting of chitosan or a salt thereof, galactose, mannose, caffeine, and any combination thereof.

58. The chewing gum of item 57, wherein the chewing gum comprises chitosan and an acid.

59. The chewing gum of item 58, wherein the chewing gum comprises a salt of chitosan.

60. The chewing gum of item 59, wherein the salt of chitosan is selected from the group consisting of chitosan lactate, chitosan acetate and chitosan hydrochloride.

61. The chewing gum of any one of item 57 to 60, wherein the chewing gum comprises a microparticle.

62. The chewing gum of item 61, wherein the microparticle comprises chitosan or a salt thereof.

63. The chewing gum of item 62, wherein the microparticle further comprises hyaluronic acid.

64. The chewing gum of any one of items 57 to 63, wherein the composition comprises a co-agglomerate of different microparticles.

65. The chewing gum of item 64, wherein the co-agglomerate comprises chitosan or a salt thereof.

66. The chewing gum of any one of items 57 to 65, wherein the chitosan or the salt thereof has a degree of deacetylation of 80% or more.

67. The composition of any one of items 57 to 66, wherein the chitosan or the salt thereof has a degree of deacetylation of from 40 to 50%.

67a. The chewing gum of item 57, wherein the chewing gum comprises sorbitols, isomalt, maltitols, xylitol, acesulfame K, sucralose, gum base, flavourings, glycerol, gum Arabic, chitosan, zinc acetate, and carnauba wax; optionally, the chewing gum may further comprise citric acid.

68. An edible composition comprising a compound selected from the group consisting of chitosan or a salt thereof.

69. The edible composition of item 68, wherein the salt is selected from the group consisting of chitosan lactate, chitosan acetate, chitosan hydrochloride, and any combination thereof.

70. The edible composition of item 68 or 69, wherein the composition is a dietary supplement, a food or a beverage.

71. The edible composition of item 70, wherein the composition is selected from the group consisting of a candy, a bonbon, a fruit gum, a chocolate, a chewing gum, and a composition for preparing a beverage (e.g. a coffee composition, a tea composition, a sherbet powder, an effervescent tablet).

72. The edible composition of item 71, wherein the beverage is selected from the group consisting of tea, coffee, juice, lemonade, milk, cola, a drinkable milk product (e.g. drinkable yoghurt), a beverage obtainable by dissolving a sherbet powder or an effervescent tablet, and an alcoholic beverage.

73. The edible composition of any one of items 68 to 72, wherein the composition comprises a microparticle, wherein the microparticle comprises the chitosan or a salt thereof.

74. The edible composition of item 73, wherein the microparticle further comprises hyaluronic acid.

75. The edible composition of any one of items 73 to 74, wherein the composition comprises a co-agglomerate of different microparticles, wherein the co-agglomerate comprises the chitosan or a salt thereof.

76. The composition of any one of items 68 to 75, wherein the chitosan or the salt thereof has a degree of deacetylation of 80% or more.

77. The composition of any one of items 68 to 75, wherein the chitosan or the salt thereof has a degree of deacetylation of from 40 to 50%.

78. A composition suitable for oral, nasal or bronchial administration, the composition comprising a microparticle, wherein the microparticle comprises chitosan or a salt thereof.

79. The composition of item 78, wherein the salt is selected from the group consisting of chitosan lactate, chitosan acetate, chitosan hydrochloride, and any combination thereof.

80. The composition of item 78 or 79, wherein the microparticle further comprises hyaluronic acid.

81. The composition of item 80, wherein the composition comprises a co-agglomerate of different microparticles, wherein the co-agglomerate comprises the chitosan or a salt thereof.

82. The composition of any one of items 78 to 81, wherein the chitosan or the salt thereof has a degree of deacetylation of 80% or more.

83. The composition of any one of items 78 to 81, wherein the chitosan or the salt thereof has a degree of deacetylation of from 40 to 50%.

84. The composition of any one of items 78 to 83, wherein the composition is selected from the group consisting of a composition for spraying or gargling, a mouth rinse, a gel for application to the oral cavity, a nose spray, a nose oil, a nose drop formulation, and a nasal irrigation formulation.

85. A sustained delivery system comprising chitosan or a salt thereof.

86. The sustained delivery system of item 85, wherein the salt is selected from the group consisting of chitosan lactate, chitosan acetate, chitosan hydrochloride, and any combination thereof.

87. The sustained delivery system of item 85 or 86, wherein the sustained delivery system is an oral or buccal sustained delivery system.

88. The sustained delivery system of any one of items 85 to 87, wherein the sustained delivery system comprises a solid or semi-solid dosage form.

89. The sustained delivery system of any one of items 85 to 88, wherein the composition is selected from the group consisting of a film, a layered structure of films, a tablet, a capsule, a gel body (e.g. a gel for oral cavity), a chewing gum, a candy, a bonbon, a fruit gum, and a chocolate.

90. The sustained delivery system of any one of items 85 to 89, wherein the composition comprises a microparticle, wherein the microparticle comprises the chitosan or a salt thereof.

91. The sustained delivery system of item 90, wherein the microparticle further comprises hyaluronic acid.

92. The sustained delivery system of any one of items 85 to 91, wherein the composition comprises a co-agglomerate of different microparticles, wherein the co-agglomerate comprises the chitosan or a salt thereof.

93. The sustained delivery system of any one of items 85 to 92, wherein the chitosan or the salt thereof has a degree of deacetylation of 80% or more.

94. The sustained delivery system of any one of items 85 to 92, wherein the chitosan or the salt thereof has a degree of deacetylation of from 40 to 50%.

95. A composition suitable for treating a surface or skin, the composition comprising a microparticle, wherein the microparticle comprises chitosan or a salt thereof.

96. The composition of item 95, wherein the salt is selected from the group consisting of chitosan lactate, chitosan acetate, chitosan hydrochloride, and any combination thereof.

97. The composition of item 95 or 96, wherein the microparticle further comprises hyaluronic acid.

98. The composition of any one of items 95 to 97, wherein the composition comprises a co-agglomerate of different microparticles, wherein the co-agglomerate comprises the chitosan or a salt thereof.

99. The composition of any one of items 95 to 98, wherein the chitosan or the salt thereof has a degree of deacetylation of 80% or more.

100. The composition of any one of items 95 to 99, wherein the chitosan or the salt thereof has a degree of deacetylation of from 40 to 50%.

101. The composition of any one of items 95 to 100, wherein the composition is selected from the group consisting of a disinfectant, a skin-caring composition (e.g. a lotion), a soap (e.g. a liquid soap), and a detergent formulation (e.g. a laundry detergent formulation or a dish detergent formulation).

102. A method of treating a surface or skin, the method comprising contacting the surface or skin with the composition of any one of items 95 to 101.

103. The method of item 102, wherein the method comprises inactivating a virus or bacterium.

104. The method of item 103, wherein the virus is a respiratory virus.

105. The method of item 104, wherein the respiratory virus is selected from the group consisting of a coronavirus, preferably a human-pathogenic coronavirus, an influenza virus, a rhinovirus, and a respiratory syncytial virus [RSV].

106. The method of item 105, wherein the human-pathogenic coronavirus is a betacoronavirus.

107. The method of item 106, wherein the human-pathogenic coronavirus is selected from the group consisting of SARS-CoV-2, MERS-CoV and SARS-CoV-1, preferably wherein the human-pathogenic coronavirus is SARS-CoV-2.

108. Use of a composition of any one of items 95 to 101 for inactivating a virus or bacterium.

109. The use of item 108, comprising contacting the surface or skin with the composition of any one of items 95 to 101.

110. The use of item 109, wherein the virus is a respiratory virus.

111. The use of item 110, wherein the respiratory virus is selected from the group consisting of a coronavirus, preferably a human-pathogenic coronavirus, an influenza virus, a rhinovirus, and a respiratory syncytial virus [RSV].

112. The use of item 111, wherein the human-pathogenic coronavirus is a betacoronavirus.

113. The use of item 111 or 121, wherein the human-pathogenic coronavirus is selected from the group consisting of SARS-CoV-2, MERS-CoV and SARS-CoV-1, preferably wherein the human-pathogenic coronavirus is SARS-CoV-2.

114. The use of any one of items 108 to 113, wherein the use is a non-medical use.

115. A method of increasing the binding of virus neutralizing antibodies, the method comprising contacting a fluid that contains a virus and virus neutralizing antibodies, wherein the virus neutralizing antibodies bind to said virus, with a compound selected from the group consisting of chitosan or a salt thereof, galactose, mannose, caffeine, and any combination thereof.

116. The method of item 115, wherein the fluid is a bodily fluid.

117. The method of item 116, wherein the bodily fluid is selected from the group consisting of saliva, blood, and a mucous secretion.

118. The method of item 117, wherein the mucous secretion is a nasal secretion or a bronchial secretion.

119. The method of any one of items 115 to 118, wherein the method further comprises contacting a mucosa with the compound.

120. The method of item 119, wherein the mucosa is selected from the group consisting of mucosa of the nose, mucosa of the pharynx, mucosa of the mouth and mucosa of the bronchia.

121. The method of any one of items 115 to 120, wherein the virus is a respiratory virus.

122. The method of item 121, wherein the respiratory virus is selected from the group consisting of a coronavirus, preferably a human-pathogenic coronavirus, an influenza virus, a rhinovirus, and a respiratory syncytial virus [RSV].

123. The method of item 122, wherein the human-pathogenic coronavirus is a betacoronavirus.

124. The method of item 122 or 123, wherein the human-pathogenic coronavirus is selected from the group consisting of SARS-CoV-2, MERS-CoV and SARS-CoV-1, preferably wherein the human-pathogenic coronavirus is SARS-CoV-2.

125. The method of any one of items 115 to 124, wherein the method is a method of treating a virus infection or treating a disease caused by a virus infection.

126. The method of item 125, wherein the disease is selected from the group consisting of COVID-19, MERS and SARS, preferably wherein the disease is COVID-19.

127. The method of any one of items 115 to 126, wherein the compound is chitosan or a salt thereof.

128. The method of item 127, wherein the salt is selected from the group consisting of chitosan lactate, chitosan acetate, chitosan hydrochloride, and any combination thereof.

129. The method of any one of items 115 to 128, comprising contacting the fluid that contains the virus and virus neutralizing antibodies with a composition that contains the compound selected from the group consisting of chitosan or a salt thereof, galactose, mannose, caffeine, and any combination thereof.

130. The method of item 129, wherein the compound is chitosan or a salt thereof.

131. The method of item 130, wherein the salt is selected from the group consisting of chitosan lactate, chitosan acetate, chitosan hydrochloride, and any combination thereof.

132. The method of any one of items 129 to 131, wherein the composition is an edible composition or a composition suitable for oral, nasal or bronchial administration.

133. The method of item 132, wherein the edible composition is a liquid or a solid composition.

134. The method of any one of items 129 to 133, wherein the composition is a dietary supplement, preferably wherein the dietary supplement is suitable for being added to a beverage, more preferably wherein the beverage is selected from the group consisting of tea, coffee, juice, lemonade, milk, cola, a drinkable milk product (e.g. drinkable yoghurt), a beverage obtainable by dissolving a sherbet powder or an effervescent tablet, and an alcoholic beverage.

135. The method of any one of items 129 to 133, wherein the composition is selected from the group consisting of a chewing gum, a candy, a bonbon, a fruit gum, a chocolate, a composition for preparing a beverage (e.g. a coffee composition, a tea composition, a sherbet powder, an effervescent tablet), and a beverage.

136. The method of item 135, wherein the composition is a chewing gum.

137. The method of item 136, wherein the chewing gum comprises chitosan and an acid.

138. The method of item 137, wherein the chewing gum comprises a salt of chitosan.

139. The method of item 138, wherein the salt of chitosan is selected from the group consisting of chitosan lactate, chitosan acetate and chitosan hydrochloride.

140. The method of item 135, wherein the beverage is selected from the group consisting of tea, coffee, juice, lemonade, milk, cola, a drinkable milk product (e.g. drinkable yoghurt), a beverage obtainable by dissolving a sherbet powder or an effervescent tablet, and an alcoholic beverage.

141. The method of any one of items 130 to 134, wherein the composition is selected from the group consisting of a solution, preferably a solution for spraying or gargling, a mouth rinse, a gel for application to the oral cavity, a nose drop formulation, a nose oil formulation, a nose spray, and a nose irrigation formulation.

142. The method of any one of items 129 to 141, wherein the composition is a sustained delivery system, preferably an oral or buccal sustained delivery system.

143. The method of any one of items 129 to 142, wherein the composition comprises a solid or semi-solid dosage form.

144. The method of item 142 or 143, wherein the composition is selected from the group consisting of a film, a layered structure of films, a tablet, a capsule, a gel body, a chewing gum, a candy, a bonbon, a fruit gum, and a chocolate.

145. The method of any one of items 129 to 144, wherein the composition comprises a microparticle.

146. The method of item 145, wherein the microparticle comprises chitosan or a salt thereof.

147. The method of item 146, wherein the microparticle further comprises hyaluronic acid.

148. The method of any one of items 145 to 147, wherein the composition comprises a co-agglomerate of different microparticles.

149. The method of item 148, wherein the co-agglomerate comprises chitosan or a salt thereof.

150. The method of any one of items 145 to 149, wherein the microparticle or the co-agglomerate is comprised in a liquid or a semi-solid composition, in particular wherein the microparticle or the co-agglomerate is suspended in a liquid or semi-solid composition.

151. The method of item 150, wherein the microparticle or the co-agglomerate is comprised, in particular suspended, in a composition for spraying or gargling, a mouth rinse, a gel for application to the oral cavity, a nose spray, a nose oil, a nose drop formulation or a nasal irrigation formulation.

152. The method of any one of items 142 to 149, wherein the delivery system comprises a drug delivery implant.

153. The method of any one of items 115 to 152, wherein the chitosan or the salt thereof has a degree of deacetylation of 80% or more.

154. The method of any one of items 115 to 153, wherein the chitosan or the salt thereof has a degree of deacetylation of from 40 to 50%.

155. The method of any one of items 115 to 154, wherein the method is carried out in vivo.

156. A compound selected from the group consisting of chitosan or a salt thereof, galactose, mannose, caffeine, and any combination thereof for use in a method of increasing the binding of virus neutralizing antibodies.

157. The compound for use of item 156, wherein the virus neutralizing antibodies bind to said virus.

158. The compound for use of item 157, wherein the use comprises contacting a fluid that contains a virus and virus neutralizing antibodies with the compound.

159. The compound for use of item 158, wherein the fluid is a bodily fluid.

160. The compound for use of item 159, wherein the bodily fluid is selected from the group consisting of saliva, blood, and a mucous secretion.

161. The compound for use of item 160, wherein the mucous secretion is a nasal secretion or a bronchial secretion.

162. The compound for use of any one of items 156 to 162, wherein the use further comprises contacting a mucosa with the compound.

163. The compound for use of item 162, wherein the mucosa is selected from the group consisting of mucosa of the nose, mucosa of the pharynx, mucosa of the mouth and mucosa of the bronchia.

164. The compound for use of any one of items 156 to 163, wherein the virus is a respiratory virus.

165. The compound for use of item 164, wherein the respiratory virus is selected from the group consisting of a coronavirus, preferably a human-pathogenic coronavirus, an influenza virus, a rhinovirus, and a respiratory syncytial virus [RSV].

166. The compound for use of item 165, wherein the human-pathogenic coronavirus is a betacorona virus.

167. The compound for use of item 165 or 166, wherein the human-pathogenic coronavirus is selected from the group consisting of SARS-CoV-2, MERS-CoV and SARS-CoV-1, preferably wherein the human-pathogenic coronavirus is SARS-Cov-2.

168. The compound for use of any one of items 156 to 167, wherein the compound is for use in treating a virus infection or treating a disease caused by a virus infection.

169. The compound for use of item 168, wherein the disease is selected from the group consisting of COVID-19, MERS and SARS, preferably wherein the disease is COVID-19.

170. The compound for use of any one of items 156 to 169, wherein the compound is chitosan or a salt thereof.

171. The compound for use of item 170, wherein the salt is selected from the group consisting of chitosan lactate, chitosan acetate, chitosan hydrochloride, and any combination thereof.

172. The compound for use of any one of items 156 to 171, wherein the use comprises using a composition that contains the compound selected from the group consisting of chitosan or a salt thereof, galactose, mannose, caffeine, and any combination thereof.

173. The compound for use of item 172, wherein the composition is as defined in any one of items 129 to 155.

174. A method of treating a patient being infected with a virus, the method comprising contacting a fluid that contains a virus and virus neutralizing antibodies, wherein the virus neutralizing antibodies bind to said virus, with a compound selected from the group consisting of chitosan or a salt thereof, galactose, mannose, caffeine, and any combination thereof, wherein said compound increases the binding of virus neutralizing antibodies.

175. The method of item 174, wherein the fluid is a bodily fluid.

176. The method of item 175, wherein the bodily fluid is selected from the group consisting of saliva, blood, and a mucous secretion.

177. The method of item 176, wherein the mucous secretion is a nasal secretion or a bronchial secretion.

178. The method of any one of items 174 to 177, wherein the virus is a respiratory virus.

179. The method of item 178, wherein the respiratory virus is selected from the group consisting of a coronavirus, preferably a human-pathogenic coronavirus, an influenza virus, a rhinovirus, and a respiratory syncytial virus [RSV].

180. The method of item 179, wherein the human-pathogenic coronavirus is a betacoronavirus.

181. The method of item 179 or 180, wherein the human-pathogenic coronavirus is selected from the group consisting of SARS-CoV-2, MERS-CoV and SARS-CoV-1, preferably wherein the human-pathogenic coronavirus is SARS-CoV-2.

182. The method of any one of items 174 to 181, wherein the method is a method of treating a virus infection or a disease caused by a virus infection.

183. The method of item 182, wherein the disease is selected from the group consisting of COVID-19, MERS and SARS, preferably wherein the disease is COVID-19.

184. The method of any one of items 174 to 183, wherein the compound is chitosan or a salt thereof.

185. The method of item 184, wherein the salt is selected from the group consisting of chitosan lactate, chitosan acetate, chitosan hydrochloride, and any combination thereof.

186. The method of any one of items 174 to 185, comprising contacting the fluid that contains the virus and virus neutralizing antibodies with a composition that contains the compound selected from the group consisting of chitosan or a salt thereof, galactose, mannose, caffeine, and any combination thereof.

187. The method of item 186, wherein the composition is as defined in any one of items 129 to 155.

The present invention is also characterized by the following items:

1. A composition for use in preventing an infection, or treating an infection, or treating a disease caused by an infection, the composition comprising a compound selected from the group consisting of chitosan or a salt thereof, galactose, mannose, caffeine, and any combination thereof.

2. The composition for use of item 1, wherein the infection is a virus, bacterial, protozoan, or mycotic infection.

3. The composition for use of item 1 or 2, wherein the infection is a virus infection.

4. The composition for use of item 2 or 3, wherein the virus is a respiratory virus.

5. The composition for use of item 4, wherein the respiratory virus is selected from the group consisting of a coronavirus, preferably a human-pathogenic coronavirus, an influenza virus, a rhinovirus, and a respiratory syncytial virus.

6. The composition for use of item 5, wherein the human-pathogenic coronavirus is a betacoronavirus.

7. The composition for use of item 5 or 6, wherein the human-pathogenic coronavirus is selected from the group consisting of SARS-CoV-2, MERS-CoV and SARS-CoV-1, preferably wherein the human-pathogenic coronavirus is SARS-CoV-2.

8. The composition for use of any one of the preceding items, wherein the disease is selected from the group consisting of COVID-19, MERS and SARS, preferably wherein the disease is COVID-19.

9. The composition for use of any one of the preceding items, wherein the use comprises contacting a bodily fluid with the composition.

10. The composition for use of item 9, wherein the bodily fluid is saliva or a mucous secretion.

11. The composition for use of any one of the preceding items, wherein the use comprises contacting a mucosa with the composition.

12. The composition for use of any one of the preceding items, wherein the use comprises contacting a skin or surface with the composition.

13. The composition for use of any one of the preceding items, wherein the use comprises contacting a wound or a burn with the composition.

14. The composition for use of any one of the preceding items, wherein the compound is chitosan or a salt thereof.

15. The composition for use of any one of the preceding items, wherein the salt is selected from the group consisting of chitosan lactate, chitosan acetate, chitosan hydrochloride, and any combination thereof.

16. The composition for use of any one of the preceding items, wherein the composition is an edible composition or a composition suitable for oral, nasal or bronchial administration.

17. The composition for use of any one of the preceding items, wherein the composition is a solid or a liquid composition.

18. The composition according to any one of the preceding items, wherein the composition is selected from the group consisting of a chewing gum, a candy, a bonbon, a fruit gum, a chocolate, and a composition for preparing a beverage (e.g. a coffee composition, a tea composition, a sherbet powder, an effervescent tablet); preferably wherein the composition is a tea composition; or preferably wherein the composition is a candy.

19. The composition for use of item 18, wherein the composition is a chewing gum.

20. The composition for use of item 19, wherein the chewing gum comprises chitosan and an acid.

21. The composition for use of item 19 or 20, wherein the chewing gum comprises a salt of chitosan.

22. The composition for use of item 21, wherein the salt of chitosan is selected from the group consisting of chitosan lactate, chitosan acetate and chitosan hydrochloride.

23. The composition for use of any one of the preceding items, wherein the composition is a dietary supplement, preferably wherein the dietary supplement is suitable for being added to a beverage, more preferably wherein the beverage is selected from the group consisting of tea, coffee, juice, lemonade, milk, cola, a drinkable milk product (e.g. drinkable yoghurt), a beverage obtainable by dissolving a sherbet powder or an effervescent tablet, and an alcoholic beverage.

24. The composition for use of any one of the preceding items, wherein the composition is a sustained delivery system, preferably an oral or buccal sustained delivery system.

25. The composition for use of any one of the preceding items, wherein the composition comprises a solid or semi-solid dosage form.

26. The composition for use of any one of the preceding items, wherein the composition comprises a particle, preferably a microparticle or a nanoparticle.

27. The composition for use of items 26, wherein the particle, preferably a microparticle or a nanoparticle, comprises chitosan or a salt thereof.

28. The composition for use of items 27, wherein the particle, preferably a microparticle or a nanoparticle, further comprises hyaluronic acid.

29. The composition for use of any one of items 26 to 28, wherein the composition comprises a co-agglomerate of particles, preferably of microparticles or nanoparticles.

30. The composition for use of item 29, wherein the co-agglomerate comprises chitosan or a salt thereof.

31. The composition for use of any one of items 26 to 30, wherein the particle, preferably a microparticle or a nanoparticle, or the co-agglomerate is comprised in a liquid or semi-solid composition, in particular wherein the particle, preferably a microparticle or a nanoparticle, or the co-agglomerate is suspended in a liquid or semi-solid composition.

32. The composition for use of item 31, wherein the particle, preferably a microparticle or a nanoparticle, or the co-agglomerate is comprised, in particular suspended, in a composition for spraying or gargling, a mouth rinse, a gel for application to the oral cavity, a nose spray, a nose oil, a nose drop formulation, or a nasal irrigation formulation.

33. The composition for use of any one items 26 to 31, wherein the particle, preferably a microparticle or nanoparticle, or the co-agglomerate is comprised in a composition suitable for treating a surface or skin.

34. The composition for use of item 33, wherein the composition is selected from the group consisting of a disinfectant, a skin-caring composition (e.g. a lotion, a cream, a gel, a spray, a salve, an ointment or a powder), a soap (e.g. a liquid soap), and a detergent formulation (e.g. a laundry detergent formulation or a dish detergent formulation).

35. The composition for use of item 33 or 34, wherein the use comprises contacting the surface or skin with the composition of any one of claims 31 to 32.

36. The composition of any one of the preceding items, wherein the chitosan or the salt thereof has a degree of deacetylation of 80% or more.

37. The composition of any one of items 1 to 35, wherein the chitosan or the salt thereof has a degree of deacetylation of from 40 to 50%.

38. Non-medical use of a composition for preventing an infection, wherein the solid composition is as defined in any one of the preceding claims.

39. The use of ites 38, wherein the infection is a virus or bacterial infection, a protozoan infection, or mycotic infection.

40. The use of item 38 or 39, wherein the infection is a virus infection.

41. The use of item 39 or 40, wherein the virus is a respiratory virus.

42. The use of item 41, wherein the virus is selected from the group consisting of a coronavirus, preferably a human-pathogenic coronavirus, an influenza virus, a rhinovirus, and a respiratory syncytial virus [RSV].

43. The use of items 42, wherein the human-pathogenic coronavirus is a betacoronavirus.

44. The use of items 42 or 43, wherein the human-pathogenic coronavirus is selected from the group consisting of SARS-CoV-2, MERS-CoV and SARS-CoV-1, preferably wherein the human-pathogenic coronavirus is SARS-CoV-2.

45. The use of any one of items 38 to 44, wherein the use comprises contacting a bodily fluid with the composition.

46. The use of items 45, wherein the bodily fluid is saliva or a mucous secretion.

47. The use of any one of items 38 to 46, wherein the use comprises contacting a mucosa with the composition.

48. The use of any one of items 38 to 47, wherein the use comprises contacting a skin or surface with the composition.

49. The use of any one of items 38 to 48, wherein the use comprises contacting a wound or a burn with the composition.

50. A method of preventing an infection, or treating an infection, or treating a disease caused by an infection by the use of a composition, wherein the solid composition is as defined in any one of items 1 to 37.

51. The method of item 50, wherein the infection is a virus or bacterial infection, a protozoan infection, or mycotic infection.

52. The method of item 50 or 51, wherein the infection is a virus infection.

53. The method of item 51 or 52, wherein the virus is a respiratory virus.

54. The method of item 53, wherein the respiratory virus is selected from the group consisting of a coronavirus, preferably a human-pathogenic coronavirus, an influenza virus, a rhinovirus, and a respiratory syncytial virus [RSV].

55. The method of item 54, wherein the human-pathogenic coronavirus is a betacoronavirus.

56. The method of item 54 or 55, wherein the human-pathogenic coronavirus is selected from the group consisting of SARS-CoV-2, MERS-CoV and SARS-CoV-1, preferably wherein the human-pathogenic coronavirus is SARS-CoV-2.

57. The method of any one of item 50 to 56, wherein the disease is selected from the group consisting of COVID-19, MERS and SARS, preferably wherein the disease is COVID-19.

58. The method of any one of item 50 to 57, wherein the method comprises contacting a bodily fluid with the composition.

59. The method of item 58, wherein the bodily fluid is saliva or a mucous secretion.

60. The method of any one of items 50 to 59, wherein the use comprises contacting a mucosa with the composition.

61. The method of any one of items 50 to 60, wherein the method comprises contacting a skin or surface with the composition.

62. The method of any one of items 50 to 61, wherein the method comprises contacting a wound or a burn with the composition.

63. A chewing gum comprising a compound selected from the group consisting of chitosan or a salt thereof, galactose, mannose, caffeine, and any combination thereof.

64. The chewing gum of item 63, wherein the chewing gum comprises chitosan and an acid.

65. The chewing gum of item 63 or 64, wherein the chewing gum comprises a salt of chitosan.

66. The chewing gum of item 65, wherein the salt of chitosan is selected from the group consisting of chitosan lactate, chitosan acetate and chitosan hydrochloride.

67. The chewing gum of item 63, wherein the chewing gum comprises sorbitols, isomalt, maltitols, xylitol, acesulfame K, sucralose, gum base, flavourings, glycerol, gum Arabic, chitosan, zinc acetate, and carnauba wax; optionally, the chewing gum may further comprise citric acid.

68. An edible composition comprising a compound selected from the group consisting of chitosan or a salt thereof.

69. The edible composition of item 68, wherein the salt is selected from the group consisting of chitosan lactate, chitosan acetate, chitosan hydrochloride, and any combination thereof.

70. The edible composition of item 68 or 69, wherein the composition is a solid or a liquid composition.

71. The edible composition of any one of items 68 to 70, wherein the composition is a dietary supplement, a food or a beverage.

72. The edible composition of item 71, wherein the composition is selected from the group consisting of a candy, a bonbon, a fruit gum, a chocolate, a chewing gum, and a composition for preparing a beverage (e.g. a coffee composition, a tea composition, a sherbet powder, an effervescent tablet); preferably wherein the composition is a tea composition; or preferably wherein the composition is a candy.

73. The edible composition of item 72, wherein the beverage is selected from the group consisting of tea, coffee, juice, lemonade, milk, cola, a drinkable milk product (e.g. drinkable yoghurt), a beverage obtainable by dissolving a sherbet powder or an effervescent tablet, and an alcoholic beverage; preferably wherein the beverage is a tea.

74. A composition suitable for oral, nasal or bronchial administration, the composition comprising a particle, preferably a microparticle or a nanoparticle, wherein the particle, preferably a microparticle or a nanoparticle, comprises chitosan or a salt thereof.

75. The composition of item 74, wherein the salt is selected from the group consisting of chitosan lactate, chitosan acetate, chitosan hydrochloride, and any combination thereof.

76. The composition of item 74 or 75, wherein the particle, preferably a microparticle or a nanoparticle, further comprises hyaluronic acid.

77. The composition of any one of items 74 to 76, wherein the composition comprises a co-agglomerate of particles, preferably of microparticles or nanoparticles, wherein the co-agglomerate comprises the chitosan or a salt thereof.

78. The composition of any one of items 74 to 77, wherein the composition is a liquid or a solid composition.

79. The composition of any one of items 74 to 78, wherein the composition is selected from the group consisting of a composition for spraying or gargling, a mouth rinse, a gel for application to the oral cavity, a nose spray, a nose oil, a nose drop formulation, and a nasal irrigation formulation.

80. A sustained delivery system comprising chitosan or a salt thereof.

81. The sustained delivery system of item 80, wherein the salt is selected from the group consisting of chitosan lactate, chitosan acetate, chitosan hydrochloride, and any combination thereof.

82. The sustained delivery system of item 80 or 81, wherein the sustained delivery system is an oral or buccal sustained delivery system.

83. The sustained delivery system of any one of items 80 to 82, wherein the sustained delivery system comprises a solid or semi-solid dosage form.

84. The sustained delivery system of any one of items 80 to 83, wherein the composition is selected from the group consisting of a film, a layered structure of films, a tablet, a capsule, a gel body (e.g. a gel for oral cavity), a chewing gum, a candy, a bonbon, a fruit gum, and a chocolate.

85. The sustained delivery system of any one of items 80 to 84, wherein the composition comprises a particle, preferably a microparticle or a nanoparticle, wherein the particle, preferably a microparticle or a nanoparticle, comprises the chitosan or a salt thereof.

86. The sustained delivery system of item 85, wherein the particle, preferably a microparticle or a nanoparticle, further comprises hyaluronic acid.

87. The sustained delivery system of any one of items 80 to 86, wherein the composition comprises a co-agglomerate of particles, preferably of microparticles or nanoparticle, wherein the co-agglomerate comprises the chitosan or a salt thereof.

88. A composition suitable for treating a surface or skin, the composition comprising a particle, preferably a microparticle or a nanoparticle, wherein the particle, preferably a microparticle or a nanoparticle, comprises chitosan or a salt thereof.

89. The composition of item 88, wherein the salt is selected from the group consisting of chitosan lactate, chitosan acetate, chitosan hydrochloride, and any combination thereof.

90. The composition of item 88 or 89, wherein the particle, preferably a microparticle or a nanoparticle, further comprises hyaluronic acid.

91. The composition of any one of items 88 to 90, wherein the composition comprises a co-agglomerate of particles, preferably of microparticles or nanoparticles, wherein the co-agglomerate comprises the chitosan or a salt thereof.

92. The composition of any one of items 88 to 91, wherein the composition is selected from the group consisting of a disinfectant, a skin-caring composition (e.g. a lotion, a cream, a gel, a spray, a salve, an ointment or a powder), a soap (e.g. a liquid soap), and a detergent formulation (e.g. a laundry detergent formulation or a dish detergent formulation).

93. A method of treating a surface or skin, the method comprising contacting the surface or skin with the composition of any one of items 88 to 92.

94. The method of item 93, wherein the method comprises inactivating a virus or bacterium, protozoan or fungus.

95. The method of item 93 or 94, wherein the method comprises inactivating a virus.

96. The method of item 94 or 95, wherein the virus is a respiratory virus.

97. The method of item 96, wherein the respiratory virus is selected from the group consisting of a coronavirus, preferably a human-pathogenic coronavirus, an influenza virus, a rhinovirus, and a respiratory syncytial virus [RSV].

98. The method of claim 97, wherein the human-pathogenic coronavirus is a betacoronavirus.

99. The method of item 97 or 98, wherein the human-pathogenic coronavirus is selected from the group consisting of SARS-CoV-2, MERS-CoV and SARS-CoV-1, preferably wherein the human-pathogenic coronavirus is SARS-CoV-2.

100. Use of a composition of any one of items 88 to 92 for inactivating a virus or bacterium, protozoan or fungus.

101. The use of item 100, comprising contacting the surface or skin with the composition of any one of items 88 to 92.

102. The use of item 100 or 101, wherein the use is for inactivating a virus.

103. The use of item 102, wherein the virus is a respiratory virus.

104. The use of item 103, wherein the respiratory virus is selected from the group consisting of a coronavirus, preferably a human-pathogenic coronavirus, an influenza virus, a rhinovirus, and a respiratory syncytial virus [RSV].

105. The use of item 104, wherein the human-pathogenic coronavirus is a betacoronavirus.

106. The use of item 103 or 104, wherein the human-pathogenic coronavirus is selected from the group consisting of SARS-CoV-2, MERS-CoV and SARS-CoV-1, preferably wherein the human-pathogenic coronavirus is SARS-CoV-2.

107. The use of any one of items 100 to 106, wherein the use is a non-medical use.

1. A method of increasing the binding of virus neutralizing antibodies, the method comprising contacting a fluid that contains a virus and virus neutralizing antibodies, wherein the virus neutralizing antibodies bind to said virus, with a compound selected from the group consisting of chitosan or a salt thereof, galactose, mannose, caffeine, and any combination thereof.

2. The method of claim 1, wherein the fluid is a bodily fluid.

3. The method of claim 2, wherein the bodily fluid is selected from the group consisting of saliva, blood, and a mucous secretion.

4. The method of claim 3, wherein the mucous secretion is a nasal secretion or a bronchial secretion.

5. The method of any one of claims 1 to 4, wherein the method further comprises contacting a mucosa with the compound.

6. The method of claim 5, wherein the mucosa is selected from the group consisting of mucosa of the nose, mucosa of the pharynx, mucosa of the mouth and mucosa of the bronchia.

7. The method of any one of claims 1 to 5, wherein the virus is a respiratory virus.

8. The method of claim 7, wherein the respiratory virus is selected from the group consisting of a coronavirus, preferably a human-pathogenic coronavirus, an influenza virus, a rhinovirus, and a respiratory syncytial virus [RSV].

9. The method of claim 8, wherein the human-pathogenic coronavirus is a betacoronavirus.

10. The method of claim 8 or 9, wherein the human-pathogenic coronavirus is selected from the group consisting of SARS-CoV-2, MERS-CoV and SARS-CoV-1, preferably wherein the human-pathogenic coronavirus is SARS-CoV-2.

11. The method of any one of claims 8 to 10, wherein the compound increases binding of SARS-CoV-2 neutralizing antibodies to the spike protein of SARS-CoV-2 or parts thereof, or to the nucleocapsid protein of SARS-CoV-2 or parts thereof.

12. The method of claim 11, wherein the RBD domain of SARS-CoV-2 or parts thereof or the nucleocapsid of SARS-CoV-2 or parts thereof is selected from the group consisting of the sequences as shown in SEQ ID NO:1, 2, 3, 4, 5, 6, and 7.

13. The method of any one of claims 1 to 12, wherein the method is for use in a method of treating a virus infection or treating a disease caused by a virus infection.

14. The method for use of claim 13, wherein the disease is selected from the group consisting of COVID-19, MERS and SARS, preferably wherein the disease is COVID-19.

15. The method for use of any one of claims 1 to 14, wherein the compound is chitosan or a salt thereof.

16. The method for use of claim 15, wherein the salt is selected from the group consisting of chitosan lactate, chitosan acetate, chitosan hydrochloride, and any combination thereof.

17. The method of any one of claims 1 to 16, comprising contacting the fluid that contains the virus and virus neutralizing antibodies with a composition that contains the compound selected from the group consisting of chitosan or a salt thereof, galactose, mannose, caffeine, and any combination thereof.

18. The method of claim 17, wherein the compound is chitosan or a salt thereof.

19. The method of claim 18, wherein the salt is selected from the group consisting of chitosan lactate, chitosan acetate, chitosan hydrochloride, and any combination thereof.

20. The method of any one of claims 17 to 19, wherein the composition is an edible composition or a composition suitable for oral, nasal or bronchial administration.

21. The method of claim 20, wherein the edible composition is a liquid or a solid composition.

22. The method of any one of claims 17 to 21, wherein the composition is a dietary supplement, preferably wherein the dietary supplement is suitable for being added to a beverage, more preferably wherein the beverage is selected from the group consisting of tea, coffee, juice, lemonade, milk, cola, a drinkable milk product (e.g. drinkable yoghurt), a beverage obtainable by dissolving a sherbet powder or an effervescent tablet, and an alcoholic beverage.

23. The method of any one of claims 17 to 21, wherein the composition is selected from the group consisting of a chewing gum, a candy, a bonbon, a fruit gum, a chocolate, a composition for preparing a beverage (e.g. a coffee composition, a tea composition, a sherbet powder, an effervescent tablet), and a beverage.

24. The method of claim 23, wherein the composition is a chewing gum.

25. The method of claim 24, wherein the chewing gum comprises chitosan and an acid.

26. The method of claim 25, wherein the chewing gum comprises a salt of chitosan.

27. The method of claim 26, wherein the salt of chitosan is selected from the group consisting of chitosan lactate, chitosan acetate and chitosan hydrochloride.

28. The method of claim 23, wherein the beverage is selected from the group consisting of tea, coffee, juice, lemonade, milk, cola, a drinkable milk product (e.g. drinkable yoghurt), a beverage obtainable by dissolving a sherbet powder or an effervescent tablet, and an alcoholic beverage.

29. The method of any one of claims 18 to 22, wherein the composition is selected from the group consisting of a solution, preferably a solution for spraying or gargling, a mouth rinse, a gel for application to the oral cavity, a nose drop formulation, a nose oil formulation, a nose spray, and a nose irrigation formulation.

30. The method of any one of claims 17 to 29, wherein the composition is a sustained delivery system, preferably an oral or buccal sustained delivery system.

31. The method of any one of claims 17 to 30, wherein the composition comprises a solid or semi-solid dosage form.

32. The method of claim 30 or 31, wherein the composition is selected from the group consisting of a film, a layered structure of films, a tablet, a capsule, a gel body, a chewing gum, a candy, a bonbon, a fruit gum, and a chocolate.

33. The method of any one of claims 17 to 32, wherein the composition comprises a particle, preferably a nano- or microparticle.

34. The method of claim 33, wherein the particle comprises chitosan or a salt thereof.

35. The method of claim 34, wherein the particle further comprises hyaluronic acid.

36. The method of any one of claims 33 to 35, wherein the composition comprises a co-agglomerate of different particles.

37. The method of claim 36, wherein the co-agglomerate comprises chitosan or a salt thereof.

38. The method of any one of claims 33 to 37, wherein the particle or the co-agglomerate is comprised in a liquid or a semi-solid composition, in particular wherein the particle or the co-agglomerate is suspended in a liquid or semi-solid composition.

39. The method of claim 38, wherein the particle or the co-agglomerate is comprised, in particular suspended, in a composition for spraying or gargling, a mouth rinse, a gel for application to the oral cavity, a nose spray, a nose oil, a nose drop formulation or a nasal irrigation formulation.

40. The method of any one of claims 30 to 37, wherein the delivery system comprises a drug delivery implant.

41. The method of any one of claims 1 to 40, wherein the chitosan or the salt thereof has a degree of deacetylation of 80% or more.

42. The method of any one of claims 1 to 41, wherein the chitosan or the salt thereof has a degree of deacetylation of from 40 to 50%.

43. The method of any one of claims 1 to 42, wherein the method is carried out in vivo.

44. A compound selected from the group consisting of chitosan or a salt thereof, galactose, mannose, caffeine, and any combination thereof for use in a method of increasing the binding of virus neutralizing antibodies.

45. The compound for use of claim 44, wherein the virus neutralizing antibodies bind to said virus.

46. The compound for use of claim 45, wherein the use comprises contacting a fluid that contains a virus and virus neutralizing antibodies with the compound.

47. The compound for use of claim 46, wherein the fluid is a bodily fluid.

48. The compound for use of claim 47, wherein the bodily fluid is selected from the group consisting of saliva, blood, and a mucous secretion.

49. The compound for use of claim 48, wherein the mucous secretion is a nasal secretion or a bronchial secretion.

50. The compound for use of any one of claims 44 to 49, wherein the use further comprises contacting a mucosa with the compound.

51. The compound for use of claim 50, wherein the mucosa is selected from the group consisting of mucosa of the nose, mucosa of the pharynx, mucosa of the mouth and mucosa of the bronchia.

52. The compound for use of any one of claims 44 to 51, wherein the virus is a respiratory virus.

53. The compound for use of claim 52, wherein the respiratory virus is selected from the group consisting of a coronavirus, preferably a human-pathogenic coronavirus, an influenza virus, a rhinovirus, and a respiratory syncytial virus [RSV].

54. The compound for use of claim 53, wherein the human-pathogenic coronavirus is a betacorona virus.

55. The compound for use of claim 53 or 54, wherein the human-pathogenic coronavirus is selected from the group consisting of SARS-CoV-2, MERS-CoV and SARS-CoV-1, preferably wherein the human-pathogenic coronavirus is SARS-Cov-2.

56. The compound for use of any one of claim 54 or 55, wherein the compound increases binding of SARS-CoV-2 neutralizing antibodies to the spike protein of SARS-CoV-2 or parts thereof, or to the nucleocapsid protein of SARS-CoV-2 or parts thereof.

57. The method of claim 56, wherein the RBD domain of SARS-CoV-2 or parts thereof or the nucleocapsid protein of SARS-CoV-2 or parts thereof is selected from the group consisting of the sequences as shown in SEQ ID NO:1, 2, 3, 4, 5, 6, and 7.

58. The compound for use of any one of claims 44 to 57, wherein the compound is for use in treating a virus infection or treating a disease caused by a virus infection.

59. The compound for use of claim 58, wherein the disease is selected from the group consisting of COVID-19, MERS and SARS, preferably wherein the disease is COVID-19.

60. The compound for use of any one of claims 44 to 59, wherein the compound is chitosan or a salt thereof.

61. The compound for use of claim 60, wherein the salt is selected from the group consisting of chitosan lactate, chitosan acetate, chitosan hydrochloride, and any combination thereof.

62. The compound for use of any one of claims 44 to 61, wherein the use comprises using a composition that contains the compound selected from the group consisting of chitosan or a salt thereof, galactose, mannose, caffeine, and any combination thereof.

63. The compound for use of claim 62, wherein the composition is as defined in any one of claims 17 to 43.

64. A compound for use in a method of treating a subject being infected with a virus, the method comprising contacting a fluid that contains a virus and virus neutralizing antibodies, wherein the virus neutralizing antibodies bind to said virus, with the compound, wherein the compound is selected from the group consisting of chitosan or a salt thereof, galactose, mannose, caffeine, and any combination thereof, wherein said compound increases the binding of virus neutralizing antibodies.

65. The compound for use of claim 64, wherein the fluid is a bodily fluid.

66. The compound for use of claim 65, wherein the bodily fluid is selected from the group consisting of saliva, blood, and a mucous secretion.

67. The compound for use of claim 66, wherein the mucous secretion is a nasal secretion or a bronchial secretion.

68. The compound for use of any one of claims 64 to 67, wherein the virus is a respiratory virus.

69. The compound for use of claim 68, wherein the respiratory virus is selected from the group consisting of a coronavirus, preferably a human-pathogenic coronavirus, an influenza virus, a rhinovirus, and a respiratory syncytial virus [RSV].

70. The compound for use of claim 69, wherein the human-pathogenic coronavirus is a betacoronavirus.

71. The compound for use of claim 69 or 70, wherein the human-pathogenic coronavirus is selected from the group consisting of SARS-CoV-2, MERS-CoV and SARS-CoV-1, preferably wherein the human-pathogenic coronavirus is SARS-CoV-2.

72. The compound for use of any one of claims 69 to 71, wherein the compound increases binding of SARS-CoV-2 neutralizing antibodies to the spike protein of SARS-CoV-2 or parts thereof, or to the nucleocapsid protein of SARS-CoV-2 or parts thereof.

73. The method of claim 72, wherein the RBD domain of SARS-CoV-2 or parts thereof or the nucleocapsid protein of SARS-CoV-2 or parts thereof is selected from the group consisting of the sequences as shown in SEQ ID NO:1, 2, 3, 4, 5, 6, and 7.

74. The compound for use of any one of claims 64 to 73, wherein the method is a method of treating a virus infection or a disease caused by a virus infection.

75. The compound for use of claim 74, wherein the disease is selected from the group consisting of COVID-19, MERS and SARS, preferably wherein the disease is COVID-19.

76. The compound for use of any one of claims 64 to 75, wherein the compound is chitosan or a salt thereof.

77. The compound for use of claim 76, wherein the salt is selected from the group consisting of chitosan lactate, chitosan acetate, chitosan hydrochloride, and any combination thereof.

78. The compound for use of any one of claims 64 to 77, comprising contacting the fluid that contains the virus and virus neutralizing antibodies with a composition that contains the compound selected from the group consisting of chitosan or a salt thereof, galactose, mannose, caffeine, and any combination thereof.

79. The compound for use of claim 78, wherein the composition is as defined in any one of claims 17 to 43.

80. An in vitro method of detecting virus-specific antibodies in a sample obtained from a subject, the method comprising contacting a fluid that contains virus-specific antibodies with virus protein under conditions that allow binding of the virus-specific antibody to the virus protein, with a compound selected from the group consisting of chitosan or a salt thereof, galactose, mannose, caffeine, and any combination thereof, thereby detecting said virus-specific antibodies.

81. The method of claim 80, wherein the virus-specific antibodies are indicative for a virus infection.

82. The method of claim 81, wherein the fluid is a bodily fluid.

83. The method of claim 82, wherein the bodily fluid is selected from the group consisting of saliva, blood, and a mucous secretion.

84. The method of claim 83, wherein the mucous secretion is a nasal secretion or a bronchial secretion.

85. The method of any one of claims 80 to 84, wherein the virus is a respiratory virus.

86. The method of claim 85, wherein the respiratory virus is selected from the group consisting of a coronavirus, preferably a human-pathogenic coronavirus, an influenza virus, a rhinovirus, and a respiratory syncytial virus [RSV].

87. The method of claim 86, wherein the human-pathogenic coronavirus is a betacoronavirus.

88. The method of claim 86 or 87, wherein the human-pathogenic coronavirus is selected from the group consisting of SARS-CoV-2, MERS-CoV and SARS-CoV-1, preferably wherein the human-pathogenic coronavirus is SARS-CoV-2.

89. The method of any one of claims 80 to 88, wherein the virus-specific protein is a SARS-CoV-2 specific protein.

90. The method of any one of claims 80 to 89, wherein the SARS-CoV-2 specific protein is the spike protein, preferably the RBD domain of the spike protein or parts thereof or to the nucleocapsid protein of SARS-CoV-2 or parts thereof.

91. The method of any one of claims 80 to 90, wherein the compound increases binding of SARS-CoV-2-specific antibodies to the spike protein of SARS-CoV-2, preferably the RBD domain of the spike protein of SARS-CoV-2 or parts thereof, or to the nucleocapsid protein of SARS-CoV-2 or parts thereof.

92. The method of any one of claims 89 to 91, wherein the RBD domain of SARS-CoV-2 or parts thereof or to the nucleocapsid protein of SARS-CoV-2 or parts thereof is selected from the group consisting of SEQ ID NO:1, 2, 3, 4, 5, 6, and 7.

93. A compound selected from the group consisting of chitosan or a salt thereof, galactose, mannose, caffeine, and any combination thereof for use in a method of detecting of virus-specific antibodies in a subject.

94. The compound for use of claim 93, wherein the method of detecting comprises contacting a fluid that contains virus-specific antibodies with a virus protein with the compound.

95. The compound for use of claim 94, wherein the fluid is a bodily fluid.

96. The compound for use of claim 95, wherein the bodily fluid is selected from the group consisting of saliva, blood, and a mucous secretion.

97. The compound for use of claim 96, wherein the mucous secretion is a nasal secretion or a bronchial secretion.

98. The compound for use of any one of claims 93 to 97, wherein the virus is a respiratory virus.

99. The compound for use of claim 98, wherein the respiratory virus is selected from the group consisting of a coronavirus, preferably a human-pathogenic coronavirus, an influenza virus, a rhinovirus, and a respiratory syncytial virus [RSV].

100. The compound for use of claim 99, wherein the human-pathogenic coronavirus is a betacorona virus.

101. The compound for use of claim 99 or 100, wherein the human-pathogenic coronavirus is selected from the group consisting of SARS-CoV-2, MERS-CoV and SARS-CoV-1, preferably wherein the human-pathogenic coronavirus is SARS-Cov-2.

102. The compound for use of any one of claims 93 to 101, wherein the compound is chitosan or a salt thereof.

103. The compound for use of claim 102, wherein the salt is selected from the group consisting of chitosan lactate, chitosan acetate, chitosan hydrochloride, and any combination thereof.

104. The compound for use of any one of claims 93 to 103, wherein the use comprises using a composition that contains the compound selected from the group consisting of chitosan or a salt thereof, galactose, mannose, caffeine, and any combination thereof.

105. The compound for use of claim 104, wherein the composition is as defined in any one of claims 17 to 19, 27, 31, 34 to 38, 41 to 42.

106. The compound for use of any one of claims 93 to 105, wherein the virus-specific protein is a SARS-CoV-2 specific protein.

107. The compound for use of any one of claims 93 to 106, wherein the SARS-CoV-2 specific protein is the spike protein, preferably the RBD domain of the spike protein or parts thereof, or to the nucleocapsid protein of SARS-CoV-2 or parts thereof.

108. The compound for use of any one of claims 93 to 107, wherein the compound increases binding of SARS-CoV-2-specific antibodies to the spike protein of SARS-CoV-2, preferably the RBD domain of the spike protein of SARS-CoV-2 or parts thereof, or to the nucleocapsid protein of SARS-CoV-2 or parts thereof.

109. The compound for use of any one of claims 93 to 108, wherein the RBD domain of SARS-CoV-2 or parts thereof or to the nucleocapsid protein of SARS-CoV-2 or parts thereof is selected from the group consisting of SEQ ID NO:1, 2, 3, 4, 5, 6, and 7.

110. A kit for the detection of virus-specific antibodies, the kit comprising a virus protein and a compound selected from the group consisting of chitosan or a salt thereof, galactose, mannose, caffeine, and any combination thereof.

111. The kit of claim 110, wherein the virus is a respiratory virus.

112. The kit of claim 111, wherein the respiratory virus is selected from the group consisting of a coronavirus, preferably a human-pathogenic coronavirus, an influenza virus, a rhinovirus, and a respiratory syncytial virus [RSV].

113. The kit of claim 112, wherein the human-pathogenic coronavirus is a betacorona virus.

114. The kit of claim 112 or 113, wherein the human-pathogenic coronavirus is selected from the group consisting of SARS-CoV-2, MERS-CoV and SARS-CoV-1, preferably wherein the human-pathogenic coronavirus is SARS-CoV-2.

115. The kit of any one of claims 110 to 114, wherein the compound is chitosan or a salt thereof.

116. The kit of claim 115, wherein the salt is selected from the group consisting of chitosan lactate, chitosan acetate, chitosan hydrochloride, and any combination thereof.

117. The kit of any one of claims 110 to 116, wherein kit comprises a composition that contains the compound selected from the group consisting of chitosan or a salt thereof, galactose, mannose, caffeine, and any combination thereof.

118. The kit of claim 117, wherein the composition is as defined in any one of claims 17 to 19, 27, 31, 34 to 38, 41 to 42.

119. The kit of any one of claims 110 to 118, wherein the kit is suitable for conducting an ELISA assay.

120. The kit of any one of claims 110 to 119, wherein the kit is suitable for the detection of SARS-CoV-2 antibodies.

121. The kit of any one of claims 110 to 120, wherein the kit is suitable for the detection of antibodies against SARS-CoV-2 spike protein, preferably the spike RBD domain or parts thereof, or to the nucleocapsid protein of SARS-CoV-2 or parts thereof.

122. The kit of any one of claims 110 to 121, wherein the compound increases binding of SARS-CoV-2-specific antibodies to the spike protein of SARS-CoV-2, preferably the RBD domain of the spike protein of SARS-CoV-2 or parts thereof, or to the nucleocapsid protein of SARS-CoV-2 or parts thereof.

123. The kit of claim 122, wherein the RBD domain of SARS-CoV-2 or parts thereof or the nucleocapsid protein of SARS-CoV-2 or parts thereof is selected from the group consisting of SEQ ID NO:1, 2, 3, 4, 5, 6, and 7.


SEQUENCE LISTING (7 sequences)

SEQ ID NO: 1 - protein, 67 aa, artificial
  SARS-CoV-2 RBD peptide sequence from position 436 to 502
WNSNNLDSKVGGNYNYLYRLFRKSNLKPFERDISTEIYQAGSTPCNGVEGFNCYFPLQSYGFQPTNG

SEQ ID NO: 2 - protein, 86 aa, Artificial
  SARS-CoV-2 RBD peptide sequence from position 426 to 511
PDDFTGCVIAWNSNNLDSKVGGNYNYLYRLFRKSNLKPFERDISTEIYQAGSTPCNGVEGFNCYFPLQSYGFQPTNGVGYQPYRVV

SEQ ID NO: 3 - protein, 1253 aa, Artificial
  SARS-CoV-2 spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGRLVPRGSPGSGYIPEAPRDGQAYVRKDGEWVLLSTFLG

SEQ ID NO: 4 - protein, 309 aa, Artificial
  SARS-CoV-2 RBD as monomer including a SNAP TAG at the N-terminus
MGLRVSIVTSHYNRTVSNQRMELVTNHTVLYNRVLLDDDKMDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLGPPDDFTGCVIAWNSNNLDSKVGGNYNYLYRLFRKSNLKPFERDISTEIYQAGSTPCNGVEGFNCYFPLQSYGFQPTNGVGYQPYRVV

SEQ ID NO: 5 - protein, 441 aa, Artificial
  SARS-CoV-2 RBD as trimer including a SNAP TAG at the N-terminus
MDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLGPPDDFTGCVIAWNSNNLDSKVGGNYNYLYRLFRKSNLKPFERDISTEIYQAGSTPCNGVEGFNCYFPLQSYGFQPTNGVGYQPYRVVPDDFTGCVIAWNSNNLDSKVGGNYNYLYRLFRKSNLKPFERDISTEIYQAGSTPCNGVEGFNCYFPLQSYGFQPTNGVGYQPYRVVPDDFTGCVIAWNSNNLDSKVGGNYNYLYRLFRKSNLKPFERDISTEIYQAGSTPCNGVEGFNCYFPLQSYGFQPTNGVGYQPYRVV

SEQ ID NO: 6 - protein, 419 aa, Artificial
  SARS-CoV-2 nucleocapsid phosphoprotein sequence
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 7 - protein, 223 aa, Artificial
  SARS-CoV-2 spike protein sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF